(19) United States
(12) Patent Application Publication
(10) Pub. No.: US 2007/0082848 A1
(43) Pub. Date: Apr. 12, 2007

(54) VEGF-C or VEGF-D materials and methods for treatment of neuropathologies

(75) Inventors: Kari Alitalo, Helsinki (FI); Marika Karkkainen, Espoo (FI); Paula Haiko, Helsinki (FI); Kirsi Sainio, Helsinki (FI); Kirmo Wartiovaara, Helsinki (FI)

Correspondence Address: MARSHALL, GERSTEIN & BORUN LLP, 233 S. WACKER DRIVE, SUITE 6300, SEARS TOWER, CHICAGO, IL, 60606, US

(73) Assignee: LICENTIA LTD., Helsinki (FI)

(21) Appl. No.: 11/600,479

(22) Filed: Nov. 16, 2006

Related U.S. Application Data
(62) Division of application No. 10/669,176, filed on Sep. 23, 2003.
Application No. 10/669,176 is a continuation-in-part of application No. 10/262,538, filed on Sep. 30, 2002.
(60) Provisional application No. 60/326,326, filed on Oct. 1, 2001.

(51) Int. Cl. A61K 38/18

(52) U.S. Cl. 514/12

(57) ABSTRACT

The present invention relates to VEGF-C or VEGF-D materials and methods for promoting growth and differentiation of neural stem cells and materials and methods for administering said cells to inhibit neuropathology.

This application is a continuation-in-part of U.S. Ser. No. 10/262,538, filed Sep. 30, 2002, which claims the benefit of U.S. Provisional Application No. 60/326,326, filed Oct. 1, 2001.

### FIELD OF THE INVENTION

The present invention provides materials and methods relating to cellular and molecular biology and medicine, particularly in the areas of vascularization and angiogenesis and the interactions of the vascular system with the nervous system.

### BACKGROUND OF THE INVENTION

Interactions of the neuropilin receptor proteins with their ligands in the collapsin/semaphorin family of molecules promotes development of neuronal growth cones and axon guidance, the process which regulates the paths of extending axons during the development of neuronal tissue. Improper retraction of the neural growth cones leads to excess, unwanted innervation of tissue.

Collapsin/semaphorin proteins belong to a family of molecules containing a characteristic semaphorin domain of approximately 500 amino acids in the amino terminus. Over 20 members of the semaphorin family are currently known, both secreted and membrane bound forms, which can be divided into six different subgroups based on primary protein structure. Both secreted and membrane bound semaphorins bind to their receptors as disulfide linked homodimers, and the cytoplasmic tail of membrane bound semaphorins can induce clustering of these ligands in the cell membrane.

Class III semaphorins, secreted proteins which contain the semaphorin domain followed by a C2-type immunoglobulin like domain, have been found to be integrally involved in the repulsion and collapse of neuronal growth cones, a process which prevents improper innervation of dorsal root ganglia, sympathetic neurons, and both cranial and spinal neurons.

Recently, two receptors for the class III semaphorins were identified, neuropilin-1(NRP-1) (Kolodkin et al., Cell. 90:753-762. 1997 and He et al. Cell. 90:739-51. 1997) and neuropilin-2 (NRP-2) (Chen et al. Neuron, 19:547, 1997). Neuropilin-1, a type-I membrane protein originally isolated from the Xenopus nervous system, was identified by semaphorin III receptor expression cloning, as a high affinity receptor for Sema III and other semaphorin family members. Further analysis by PCR using sequences homologous to neuropilin-1 identified a related receptor, neuropilin-2, which shows approximately 44% homology to NRP-1 throughout the entire protein length.

The extracellular portion of both NRP-1 and NRP-2 shows an interesting mix of cell binding domains, possessing five distinct protein domains designated a1/a2, b1/b2, and c. The a1/a2 (CUB) domains resemble protein sequences found in complement components C1r and Cs while the b1/b2 domains are similar to domains found in coagulation factors V and VIII. The central portion of the c domain, similar to the meprin/A5/mu-phosphotase (MAM) homology domain, is important for neuropilin dimerization. The intracellular region of neuropilins contains a transmembrane domain and a short, highly conserved cytoplasmic tail of 43 amino acids that possesses no known catalytic activity to date. Both the a1/a2 and b1/b2 domains are necessary to facilitate semaphorin binding to neuropilins.

Since the short cytoplasmic tail of neuropilins does not possess signaling capabilities, neuropilins probably couple with other receptors to transmit intracellular signals as a result of semaphorin binding. Investigation of this scenario concluded that neuropilins interact with another family of semaphorin receptors, the plexins, which possess a cytoplasmic tail containing a sex-plexin domain capable of undergoing phosphorylation and initiating downstream signaling cascades (Tamagnone et al., Trends in Cell Biol., 10:377-83. 2000). Plexins were originally isolated as orphan receptors for membrane bound semaphorins, and plexins alone are incapable of binding secreted semaphorins such as those in the class III subfamily. A great deal of evidence now demonstrates that class III semaphorin binding is mediated through a receptor complex which includes homo- or heterodimeric neuropilins and a plexin molecule needed to transduce intracellular signals. Interactions of plexins with neuropilins confer specificity of semaphorin binding and can also increase the binding affinity of these ligands. Signaling of semaphorins through their receptors is reviewed in Fujisawa et al., (Current Opinion in Neurobiology. 8:587.1998) and Tamagnone et al., (Trends in Cell Biol., 10:377, 2000).

Neuropilin-1 (Tagaki et al., Neutron 7:295-307. 1991: Fujisawa et al., Cell Tissue Res. 290:465-70, 1997), a 140 kD protein whose gene is localized to chromosome 10p12 (Rossingnol et al. Genomics 57:459-60, 1999), is expressed in a wide variety of tissues during development, including nervous tissue, capillaries and vessels of the cardiovascular system, and skeletal tissue, and persists in many adult tissues, most notably the placenta and heart. In addition to binding Sema3A. NRP-1 also binds several other semaphorin family members including Sema3B. Sema3C (SemaE), and Sema3F (SemaIV) (with low affinity) (He et al., Cell 90:739-51, 1997; Kolodkin et al., Cell 90:753-62. 1997). Mice homozygous mutant at the NRP-1 locus demonstrate defects not only in axonal guidance but also show altered vascularization in the brain and defects in the formation of large vessels of the heart (Kawasaki et al, Development 126:4895. 1990). Interestingly, NRP-1 overexpression in embryos leads to excess capillary and vessel formation and hemorrhaging, implicating a role for NRP-1 in vascular development (Kitsukawa et al, Development, 121:4309, 1995).

Recent evidence shows that neuropilin-1 can act as a receptor for an isoform of vascular endothelial growth factor (VEGF/VEGF-A) (Soker et al, Cell 92:735. 1998), which is a key mediator of vasculogenesis and angiogenesis in embryonic development (reviewed in Robinson et al., J Cell Science, 114:853-65) and also plays a significant role in tumor angiogenesis. Binding of VEGF to receptor tyrosine kinases (RTK) VEGFR-1 and VEGFR-2 facilitates vascular development. Both the non-heparin dependent VEGF₁₂₁ isoform and the heparin-binding VEGF₁₆₅ bind VEGFR-2 with the same affinity in vitro, but do not elicit equivalent biochemical responses, indicating that additional factors mediate VEGFR-2 activation (Whitaker et al., J Bio Chem. 276:25520-31, 2001). Analysis of the binding of several splice variants of VEGF reveal that NRP-1 does not bind the VEGF₁₂₁ isoform but selectively binds the VEGF₁₆₅ variant in a heparin-dependent manner within the b domain of NRP-1 (Giger et al., Neuron 21:1079-92. 1998). NRP-1 demonstrates a binding affinity for the VEGF₁₆₅ isoform comparable to that of its Sema3A ligand. This differential affinity of NRP-1 for VEGF₁₆₅ may explain the signaling capabilities of this splice variant over the non-heparin binding VEGF₁₂₁ and may indicate that neuropilin-1 interacts with VEGFR-2 as a co-receptor in VEGF binding (Whitaker et al., 2001), similar to its role in plexin/semaphorin complexes. VEGF₁₆₅ binds NRP-1 through VEGF exon 7, which confers heparin binding affinity to this molecule, and is lacking in the VEGF₁₂₁ isoform. NRP-1 also binds other VEGF family members. VEGF-B (Migdal et al. J. Biol. Chem. 273:22272-78. 1998), placenta growth factor (PIGF-2) (Makinen et al., J. Biol. Chem. 274: 21217-222. 1999) and VEGF-C (international Patent Publ. WO00/23565).

Neuropilin-2 (Chen et al., Neuron 19:547-59. 1997), a 120 kD protein whose gene is localized to chromosome 2q34 (Rossingnol et al. Genomics 57:459-60. 1999), exhibits similar tissue distribution in the developing embryo as neuropilin-1, but does not appear to be expressed in endothelial cells of blood capillaries (Chen et al., Neuron 19:547-59. 1997), but is expressed in lymphatic capillaries. NRP-2 is also a semaphorin receptor, binding Sema3F with high affinity, Sema3C with affinity comparable to Sema3C/NRP-1 binding, NRP-2 also appears to interact with very low affinity to Sema3A (Kolodkin et al., Cell 90:753-62. 1997). NRP-2 deficient mice exhibit defects in the Sema3F-dependent formation of sympathetic and hippocampal neurons and defects in axonal projections in the peripheral and central nervous systems, implicating NRP-2 in axonal guidance (Chen et al., Neuron 25:43-56. 2000; Giger et al., Neuron 25:29-41. 2000) and suggesting distinct roles for NRP-1 and NRP-2 in development. NRP-2 knock-out mice demonstrated an absence or severe reduction of small lymphatic vessels and capillaries during development while arteries, veins and larger lymphatic vessels were normal, suggesting that NRP-2 is required for the development of small lymphatic vessels and capillaries (Yuan et al., Development 129:4797-806. 2002). NRP-2 expression has also been noted in sites that innervate smooth muscle cells such as mesentery, muscular, and submucosal plexuses (Cohen et al., Biochem. Biophy. Res. Comm. 284:395-403. 2001).

Experimental evidence establishes that, similar to NRP-1, neuropilin-2 preferentially binds VEGF₁₆₅, and shows additional binding to the VEGF₁₄₅ isoform, another heparin-binding splice variant of VEGF (Gluzman-Poltorak et al., J. Biol Chem. 275:18040-45. 2000). Neuropilin-2 interaction with the VEGF₁₄₅ splice variant, which lacks exon 7, is mediated through VEGF₁₄₅ exon 6 which, like exon 7, is capable of mediating heparin binding activity. VEGF₁₄₅ cannot bind NRP-1, which further supports the theory of differential functions for neuropilin-1 and neuropilin-2 in vascular development. VEGF₁₄₅ was originally isolated from carcinomas of the female reproductive tract (Pavelock et al. Endocrinology, 142: 613-22. 2001) where neuropilin-2 expression shows differential regulation in response to hormonal changes as compared to NRP-1 and VEGFR-2. The co-expression of both neuropilins, VEGFs, and VEGFRs in a particular cell type may be indicative of a potential receptor/ligand complex formation and needs to be investigated in greater detail.

VEGF/VEGFR interactions play an integral role in embryonic vasculogenesis and angiogenesis, as well as a role in adult tissue neovascularization during wound healing, remodeling of the female reproductive system, and tumor growth. Elucidating additional factors involved in the regulation of neovascularization and angiogenesis, as well as their roles in such processes, would aid in the development of therapies directed toward prevention of vascularization of solid tumors and induction of tumor regression, and induction of vascularization to promote faster, more efficient wound healing after injury, surgery, or tissue transplantation, or to treat ischemia by inducing angiogenesis and arteriogenesis of vessels that nourish the ischemic tissue. In fact, modulation of angiogenic processes may be instrumental in treatment or cure of many of the most significant diseases that plague humans in the developed world, such as cerebral infarction/bleeding, acute myocardial infarction and ischemia, and cancers.

Modulation of neuronal growth also is instrumental in treatment of numerous congenital, degenerative, and trauma-related neurological conditions. The newfound interaction between neuropilins and VEGF provides one target for intervention at a molecular level for both neuronal and vascular diseases and conditions. However, the ability to develop targeted therapies is complicated by the existence of multiple binding partners for neuropilins. There exists a need to delineate molecules that bind neuropilins in order to permit identification of modulation of neuropilin activities and to optimize the specificity of such molecules to optimize therapies in areas of unwanted angiogenesis, as in cancers or solid tumor growth, and potentiate pro-angiogenic properties to promote and speed needed blood vessel growth, as in wound healing; and optimize therapies directed to neuronal growth and organization.

### SUMMARY OF THE INVENTION

The present invention addresses one or more needs in the art relating to modulation of angiogenic and nervous system growth and function, by identifying novel molecular interactions between neuropilins and VEGF-C molecules, and between neuropilins and VEGFR-3 molecules. These newly delineated interactions facilitate identification of novel materials and methods for modulating both angiogenic processes (including lymphangiogenic processes) and processes involved in neural cell growth, differentiation, and regeneration. The newly delineated interactions also facilitate better therapeutic targeting by permitting design of molecules that modulate single receptor-ligand interactions highly selectively, or molecules that modulate multiple interactions.

For example, the discovery of VEGF-C-neuropilin interactions provides novel screening assays to identify new therapeutic molecules to modulate (up-regulate/activate/stimulate or downregulate/inhibit) VEGF-C-neuropilin interactions. Such molecules are useful as therapeutics (and/or as lead compounds) for diseases and conditions in which VEGF-C/neuropilin interactions have an influence, including those in which lymphatic or blood vessel growth play a role, or nervous system diseases and conditions.

In one embodiment, the invention provides a method for identifying a modulator of binding between a neuropilin receptor and VEGF-C polypeptide comprising steps of:

a) contacting a neuropilin composition that comprises a neuropilin polypeptide with a VEGF-C composition that comprises a VEGF-C polypeptide, in the presence and in the absence of a putative modulator compound;

b) detecting binding between neuropilin polypeptide and VEGF-C polypeptide in the presence and absence of the putative modulator; and

c) identifying a modulator compound based on a decrease or increase in binding between the neuropilin polypeptide and the VEGF-C polypeptide in the presence of the putative modulator compound, as compared to binding in the absence of the putative modulator compound.

In one variation, the method further includes a step (d) of making a modulator composition by formulating a modulator identified according to step (c) in a carrier, preferably a pharmaceutically acceptable carrier. A modulator so formulated is useful in animal studies and also as a therapeutic for administration to image tissues or treat diseases associated with neuropilin-VEGF-C interactions, wherein the administration of a compound could interfere with detrimental activity of these molecules, or promote beneficial activity. Thus, in still another variation, the method further includes a step (e) of administering the modulator composition to an animal that comprises cells that express the neuropilin receptor, and determining physiological effects of the modulator composition in the animal. The animal may be human, or any animal model for human medical research, or an animal of importance as livestock or pets. In a preferred variation, the animal (including humans) has a disease or condition characterized by aberrant neuropilin-2/VEGF-C biology, and the modulator improves the animal's state (e.g., by reducing disease symptoms, slowing disease progression, curing the disease, or otherwise improving clinical outcome).

Step (a) of the foregoing methods involves contacting a neuropilin composition with a VEGF-C composition in the presence and absence of a compound. By “neuropilin composition” is meant any composition that includes a whole neuropilin receptor polypeptide, or includes at least the portion of the neuropilin polypeptide needed for the particular assay—in this case the portion of the neuropilin polypeptide involved in VEGF-C binding. Exemplary neuropilin compositions include: (i) a composition comprising a purified polypeptide that comprises an entire neuropilin protein or that comprises a neuropilin receptor extracellular domain fragment that binds VEGF-C polypeptides; (ii) a composition containing phospholipid membranes that contain neuropilin receptor polypeptides on their surface; (iii) a living cell recombinantly modified to express increased amounts of a neuropilin receptor polypeptide on its surface (e.g., by inserting a neuropilin gene, preferably with an attached promoter, into a cell; or by amplifying an endogenous neuropilin gene; or by inserting an exogenous promoter or other regulatory sequence to up-regulate an endogenous neuropilin gene); and (iv) any isolated cell or tissue that naturally expresses the neuropilin receptor polypeptide on its surface. For certain assay formats, it may be desirable to bind the neuropilin molecule of interest (e.g., a composition comprising a polypeptide comprising a neuropilin receptor extracellular domain fragment) to a solid support such as a bead or assay plate well. “Neuropilin composition” is intended to include such structures as well. Likewise, fusion proteins are contemplated w % herein the neuropilin polypeptide is fused to another protein (such as an antibody Fc fragment) to improve solubility, or to provide a marker epitope, or serve any other purpose. For other assay formats, soluble neuropilin peptides may be preferred. In one preferred variation, the neuropilin composition comprises a polypeptide comprising a neuropilin receptor extracellular domain fragment fused to an immunoglobulin Fc fragment. Although two family members are currently known, neuropilin-1 and neuropilin-2, practice of the invention with other neuropilin receptor family members that are subsequently discovered is contemplated. The neuropilin receptor chosen is preferably of vertebrate origin, more preferably mammalian, still more preferably primate, and still more preferably human. And, while it will be apparent that the assay will likely give its best results if the functional portion of the chosen neuropilin receptor is identical in amino acid sequence to the native receptor, it will be apparent that the invention can still be practiced if variations have been introduced in the neuropilin sequence that do not eliminate its VEGF-C binding properties. Use of variant sequences with at least 90%, 95%, 96%, 97%, 98%, or 99% amino acid identity is specifically contemplated.

VEGF-C molecules occur naturally as secreted factors that undergo several enzymatic cleavage reactions before release into the surrounding milieu. Thus, “VEGF-C composition” means any composition that includes a prepro-VEGF-C polypeptide, the intermediate and final cleavage products of prepro-VEGF-C, ΔNΔC VEGF-C, or includes at least the portion of the VEGF-C needed for the particular assay—in this case the portion involved in binding to a neuropilin receptor. Exemplary VEGF-C compositions include: (i) a composition comprising purified complete prepro-VEGF-C polypeptide or comprising a prepro-VEGF-C polypeptide fragment that binds the neuropilin receptor chosen for the assay; and (ii) conditioned media from a cell that secretes the VEGF-C protein. For certain assay formats, it may be desirable to bind the VEGF-C molecule of interest (e.g., a polypeptide comprising VEGF-C fragment) to a solid support such as a bead or assay plate well. “VEGF-C composition” is intended to include such structures as well. Likewise, fusion proteins are contemplated. The data provided herein establishes that isoforms of VEGF-C bind both neuropilin-1 and neuropilin-2. The VEGF-C polypeptide chosen is preferably of vertebrate origin, more preferably mammalian, still more preferably primate, and still more preferably human. In one embodiment the VEGF-C compositions comprises a fragment of human prepro-VEGF-C that contains amino acids 103-227 of SEQ. ID NO.: 24. In another embodiment, the VEGF-C composition comprises amino acids 32-227 of the human prepro-VEGF-C sequence of SEQ. ID NO.: 24. While it will be apparent that the assay will likely give its best results if the functional portion of the chosen VEGF-C is identical in amino acid sequence to the corresponding portion of the native VEGF-C, it will be apparent that the invention can still be practiced if variations have been introduced in the VEGF-C sequence that do not eliminate its neuropilin receptor binding properties. Use of variant sequences with at least 90%, 95%, 96%, 97%, 98%, or 99% amino acid identity is specifically contemplated.

The putative modulator compound that is employed in step (a) can be any organic or inorganic chemical or biological molecule or composition of matter that one would want to test for ability to modulate neuropilin-VEGF-C interactions. Since the most preferred modulators will be those that can be administered as therapeutics, it will be apparent that molecules with limited toxicity are preferred. However, toxicity can be screened in subsequent assays, and can be “designed out” of compounds by pharmaceutical chemists. Screening of chemical libraries such as those customarily kept by pharmaceutical companies, or combinatorial libraries, peptide libraries, and the like is specifically contemplated.

Step (b) of the above-described method includes detecting binding between neuropilin and VEGF-C in the presence and absence of the compound. Any technique for detecting intermolecular binding may be employed. Techniques that provide quantitative measurements of binding are preferred. For example, one or both of neuropilin/VEGF-C may comprise a label, such as a radioisotope, a fluorophore, a fluorescing protein (e.g., natural or synthetic green fluorescent proteins), a dye, an enzyme or substrate, or the like. Such labels facilitate quantitative detection with standard laboratory machinery and techniques. Immunoassays represent a common and highly effective body of techniques for detecting binding between two molecules.

When the neuropilin composition comprises a cell that expresses neuropilin naturally or recombinantly on its surface, it will often be possible to detect VEGF-C binding indirectly, e.g., by detecting or measuring a VEGF-C binding-induced physiological change in the cell. Such possible changes include phosphorylation of the neuropilin associated VEGF-receptor; cell chemotaxis: cell growth; DNA synthesis; changes in cellular morphology; ionic fluxes; or the like.

Step (c) of the outlined method involves identifying a modulator compound on the basis of increased or decreased binding between the neuropilin receptor polypeptide and the VEGF-C polypeptide in the presence of the putative modulator compound as compared to such binding in the absence of the putative modulator compound. Generally, more attractive modulators are those that will activate or inhibit neuropilin-VEGF-C binding at low concentrations, thereby permitting use of the modulators in a pharmaceutical composition at lower effective doses.

In another embodiment, the invention provides a method for screening for selectivity of a modulator of VEGF-C biological activity. The term “selectivity”—when used herein to describe modulators—refers to the ability of a modulator to modulate one protein-protein interaction (e.g., VEGF-C binding with neuropilin-2) with minimal effects on the interaction of another protein-protein interaction of one or more of the binding pairs (e.g., VEGF-C binding with VEGFR-2, or VEGFR-3, or neuropilin-1). More selective modulators significantly alter the first protein-protein interaction with minimal effects on the other protein-protein interaction, whereas non-selective modulators will alter two or more protein-protein interactions. It will be appreciated that selectivity is of immense interest to the design of effective pharmaceuticals. For example, in some circumstances, it may be desirable to identify modulators that alter VEGF-C/neuropilin interactions but not semaphorin/neuropilin interactions, because one wishes to modulate vessel growth but not neurological growth. Alternatively, it may be desirable to use a selective modulator to modulate neuronal growth. It may be desirable in some circumstances to non-selectively inhibit all VEGF-C related activities, e.g., in anti-tumor therapy. The molecular interactions identified herein permit novel screening assays to help identify the selectivity of modulators.

For example, VEGF-C molecules are also known ligands for the VEGFR-2 and VEGFR-3 tyrosine kinase receptors. VEGF-C/VEGFR-3 interactions appear to be integrally involved in the development and maintenance of lymphatic vasculature and may also be involved in cancer metastasis through the lymphatic system. In one instance it may be beneficial to modulate VEGF-C/neuropilin interactions specifically while in another instance it may be useful to selectively modulate the VEGF-C/VEGFR interactions. The present invention provides counterscreen assays that identify the selectivity of a modulator for neuropilin-VEGF-C binding or VEGF-C-VEGFR binding.

Thus, in one variation, the invention provides a method, comprising steps of:

a) contacting a VEGF-C composition with a neuropilin composition in the presence and in the absence of a compound and detecting binding between the VEGF-C and the neuropilin (in the compositions) in the presence and absence of the compound, wherein differential binding in the presence and absence of the compound identifies the compound as a modulator of binding between the VEGF-C and the neuropilin;

b) contacting a VEGF-C composition with a composition comprising a VEGF-C binding partner in the presence and in the absence of the compound and detecting binding between the VEGF-C and the binding partner in the presence and absence of the compound, wherein differential binding in the presence and absence of the compound identifies the compound as a modulator of binding between the VEGF-C and the binding partner; and wherein the binding partner is selected from the group consisting of:
(i) a polypeptide comprising a VEGFR-3 extracellular domain; and
(ii) a polypeptide comprising a VEGFR-2 extracellular domain; and

(c) identifying the selectivity of the modulator compound in view of the binding detected in steps (a) and (b).

Step (a) of the above embodiment involves contacting a neuropilin composition with a VEGF-C composition as described previously. Step (b) of the outlined method involves contacting a VEGF-C composition as described in step (a) with a composition comprising a VEGF-C binding partner in the presence and in the absence of the same compound. The VEGF-C binding partner is selected from the group consisting of: (i) a polypeptide comprising a VEGFR-3 extracellular domain; and (ii) a polypeptide comprising a VEGFR-2 extracellular domain. Thus, the above-described embodiment involves measuring selectivity of a modulator of VEGF-C/neuropilin binding in relation to VEGF-C binding to its receptors, VEGFR-2 and VEGFR-3. The VEGF-C binding partner chosen is preferably of vertebrate origin, more preferably mammalian, still more preferably primate, and still more preferably human. And, while it will be apparent that the assay will likely give its best results if the functional portion of the chosen VEGF-C binding partner is identical in amino acid sequence to the native VEGF-C binding partner, it will be apparent that the invention can still be practiced if variations have been introduced in the VEGF-C binding partner sequence that do not eliminate its VEGF-C binding properties. Use of variant sequences with at least 90%, 95%, 96%, 97%, 98%, or 99% amino acid identity is specifically contemplated. Any technique for detecting intermolecular binding may be employed. For example, one or both of the binding partner or the VEGF-C may comprise a label, such as a radioisotope, a fluorophore, a fluoresceing protein (e.g., natural or synthetic green fluorescent proteins), a dye, an enzyme or substrate, or the like. Such labels facilitate detection with standard laboratory machinery and techniques.

In one variation, the binding partner composition comprises a cell that expresses the binding partner naturally or recombinantly on its surface. In this situation, it will often be possible to detect VEGF-C binding indirectly, e.g., by detecting or measuring a VEGF-C binding-induced physiological change in the cell. Such possible changes include phosphorylation of the associated VEGFR; cell chemotaxis; cell growth, changes in cellular morphology; ionic fluxes, or the like.

Step (c) of the outlined method involves identifying the selectivity of the modulator compound on the basis of increased or decreased binding in steps (a) and (b). A compound that is a selective modulator causes significant differential binding in either step (a) or step (b), but does not cause significant differential binding in both steps (a) and (b). A non-specific modulator causes significant differential binding in both steps (a) and (b).

In still another embodiment, the invention provides a method for screening for selectivity of a modulator of neuropilin biological activity, comprising steps of:

a) contacting a neuropilin composition with a VEGF-C composition in the presence and in the absence of a compound and detecting binding between the neuropilin and the VEGF-C in the presence and absence of the compound, wherein differential binding in the presence and absence of the compound identifies the compound as a modulator of binding between the neuropilin and the VEGF-C:

b) contacting a neuropilin composition with a composition comprising a neuropilin binding partner in the presence and in the absence of the compound and detecting binding between the neuropilin and the binding partner in the presence and absence of the compound, wherein differential binding in the presence and absence of the compound identifies the compound as a modulator of binding between the neuropilin and the binding partner; and wherein the binding partner is selected from the group consisting of:
(i) a polypeptide comprising an amino acid sequence of a semaphorin 3 polypeptide,
(ii) a polypeptide comprising a VEGF-A amino acid sequence, a VEGF-B amino acid sequence, a VEGF-D amino acid sequence, a PIGF-2 amino acid sequence, a VEGFR-1 amino acid sequence, a VEGFR-2 amino acid sequence, a VEGFR-3 amino acid sequence; and
(iii) a polypeptide comprising an amino acid sequence of a plexin polypeptide

d) identifying the selectivity of the modulator compound in view of the binding detected in steps (a) and (b).

Step (a) of the above embodiment involves contacting a neuropilin composition with a VEGF-C composition as described previously. Step (b) of the outlined method involves contacting a neuropilin composition as described in step (a) with a composition comprising a neuropilin binding partner in the presence and in the absence of a compound. The neuropilin binding partner comprises any protein other than VEGF-C that the neuropilin binds. Exemplary binding partners include the following polypeptides: a polypeptide comprising the amino acid sequence of a semaphorin 3 family member polypeptide; a polypeptide comprising a VEGF-A amino acid sequence, a polypeptide comprising a VEGF-B amino acid sequence, a polypeptide comprising a VEGF-D amino acid sequence, a polypeptide comprising a PIGF-2 amino acid sequence, a polypeptide comprising a VEGFR-1 amino acid sequence, a polypeptide comprising a VEGFR-1 amino acid sequence, a polypeptide comprising a VEGFR-3 amino acid sequence; and a polypeptide comprising the amino acid sequence of a plexin family member. The binding partners chosen are preferably of vertebrate origin, more preferably mammalian, still more preferably primate, and still more preferably human. And, while it will be apparent that the assay will likely give its best results if the functional portion of the chosen neuropilin binding partner is identical in amino acid sequence to the native sequence, it will be apparent that the invention can still be practiced if variations have been introduced in the native sequence that do not eliminate its neuropilin binding properties. Use of variant sequences with at least 90%, 95%, 96%, 97%, 98%, or 99% amino acid identity is specifically contemplated.

The above-described method includes detecting binding between the neuropilin composition and the binding partner in the presence and absence of the compound. Any technique for detecting intermolecular binding may be employed. For example, one or both of the binding partner or the neuropilin may comprise a label, such as a radioisotope, a fluorophore, a fluorescing protein (e.g., natural or synthetic green fluorescent proteins), a dye, an enzyme or substrate, or the like. Such labels facilitate detection with standard laboratory machinery and techniques.

Step (c) of the outlined method involves identifying the selectivity of the modulator compound on the basis of increased or decreased binding in steps (a) and (b), and having the characteristics of a selective modulator compound as described previously.

In an additional embodiment, the invention provides a method of screening for modulators of binding between a neuropilin growth factor receptor and a VEGFR-3 polypeptide comprising steps of:

a) contacting a neuropilin composition with a VEGFR-3 composition in the presence and in the absence of a putative modulator compound;

b) detecting binding between the neuropilin and the VEGFR-3 in the presence and absence of the putative modulator compound; and

c) identifying a modulator compound based on a decrease or increase in binding between the neuropilin composition and the VEGFR-3 composition in the presence of the putative modulator compound, as compared to binding in the absence of the putative modulator compound.

Step (a) of the aforementioned method involves contacting a neuropilin composition as described with a VEGFR-3 composition in the presence and absence of a putative modulator compound. The neuropilin composition contemplated is described previously. A “VEGFR-3 composition” comprises a member selected from the group consisting of (i) a composition comprising a purified polypeptide that comprises an entire VEGFR-3 protein or that comprises a VEGFR-3 fragment that binds the neuropilin; (ii) a composition containing phospholipid membranes that contain VEGFR-3 polypeptides on their surface; (iii) a living cell recombinantly modified to express increased amounts of a VEGFR-3 on its surface; and (iv) any isolated cell or tissue that naturally expresses the VEGFR-3 on its surface. For certain assay formats, it may be desirable to bind the VEGFR-3 molecule of interest (e.g., a polypeptide comprising a VEGFR-3 extracellular domain fragment) to a solid support such as a bead or assay plate well. “VEGFR-3 composition” is intended to include such structures as well. Likewise, fusion proteins are contemplated. For other assay formats, soluble VEGFR-3 peptides may be preferred. In one preferred variation, the VEGFR-3 receptor composition comprises a VEGFR-3 receptor fragment fused to an immunoglobulin Fc fragment.

Step (b) of the above method involves detecting binding between the neuropilin composition and the VEGFR-3 composition in the presence and absence of the compound. Any technique for detecting intermolecular binding may be employed. For example, one or both of neuropilin/VEGFR-3 may comprise a label, such as a radioisotope, a fluorophore, a fluorescing protein (e.g., natural or synthetic green fluorescent proteins), a dye, an enzyme or substrate, or the like. Such labels facilitate detection with standard laboratory machinery and techniques.

Generally, more attractive modulators are those that will activate or inhibit neuropilin-VEGFR-3 binding at lower concentrations, thereby permitting use of the modulators in a pharmaceutical composition at lower effective doses.

In another embodiment, the invention provides for a method for screening for selectivity of a modulator of VEGFR-3 biological activity, comprising steps of:

a) contacting a VEGFR-3 composition with a neuropilin composition in the presence and in the absence of a compound and detecting binding between the VEGFR-3 and the neuropilin in the presence and absence of the compound, wherein differential binding in the presence and absence of the compound identifies the compound as a modulator of binding between the VEGFR-3 and the neuropilin;

b) contacting a VEGFR-3 composition with a composition comprising a VEGFR-3 binding partner in the presence and in the absence of a compound and detecting binding between the VEGFR-3 and the binding partner in the presence and absence of the compound, wherein differential binding in the presence and absence of the compound identifies the compound as a modulator of binding between the VEGFR-3 and the binding partner; and wherein the binding partner is selected from the group consisting of:
(i) a polypeptide comprising a VEGF-C polypeptide; and
(ii) a polypeptide comprising a VEGF-D polypeptide; and

c) identifying the selectivity of the modulator compound in view of the binding detected in steps (a) and (b).

A selective modulator causes significant differential binding in either step (a) or step (b), but does not cause significant differential binding in both steps (a) and (b).

It will be apparent that the foregoing selectivity screens represent only a portion of the specific selectivity screens of the present invention, because the neuropilins, VEGF-C, VEGF-D, and VEGFR-3 all have multiple binding partners, creating a number of permutations for selectivity screens. Any selectivity screen that involves looking at one of the following interactions: (i) neuropilin-1/VEGF-C;; (iii) neuropilin-2/VEGF-C;; (v) neuropilin-1/VEGFR-3; and (vi) neuropilin-2/VEGFR3; together with at least one other interaction (e.g., a known interaction of one of these molecules, or a second interaction from the foregoing list) is specifically contemplated as part of the present invention.

Likewise, all of the screens for modulators and the selectivity screens optionally comprising one or both of the following steps: (1) making a modulator composition by formulating a chosen modulator in a pharmaceutically acceptable carrier; and (2) administering the modulator so formulated to an animal or human and determining the effect of the modulator. Preferably, the animal or human has a disease or condition involving one of the foregoing molecular interactions and the animal or human is monitored to determine the effect of the modulator on the disease or condition, which, hopefully, is ameliorated or cured.

The discovery of neuropilin-2 and neuropilin-1 binding to VEGF-C molecules provides new and useful materials and methods for investigating biological processes involved in many currently known disease states. For example, the invention provides for a method of modulating growth, migration, or proliferation of cells in a mammalian organism, comprising a step of:

(a) identifying a mammalian organism having cells that express a neuropilin receptor; and

(b) administering to said mammalian organism a composition, said composition comprising a neuropilin polypeptide or fragment thereof that binds to a VEGF-C polypeptide;

wherein the composition is administered in an amount effective to modulate growth, migration, or proliferation of cells that express neuropilin in the mammalian organism. Administration of soluble forms of the neuropilin is preferred.

Preferably, the mammalian organism is human. Also, the cells preferably comprise vascular endothelial cells, especially cells of lymphatic origin, such as human microvascular endothelial cells (HMVEC) and human cutaneous fat pad microvascular cells (HUCEC). In a highly preferred embodiment, the organism has a disease characterized by aberrant growth, migration, or proliferation of endothelial cells. The administration of the agent beneficially alters the aberrant growth, migration, or proliferation, e.g., by correcting it, or reducing its severity, or reducing its deleterious symptoms or effects.

For example, in one variation, the animal has a cancer, especially a cancerous tumor characterized by vasculature containing neuropilin-expressing endothelial cells. A composition is selected that will decrease growth, migration, or proliferation of the cells, and thereby retard the growth of the tumor by preventing growth of new vasculature. In such circumstances, one may wish to administer agents that inhibit other endothelial growth factor/receptor interactions, such as inhibitors of the VEGF-family of ligands; endostatins; inhibitory angiopoietins, or the like. Exemplary inhibitors include antibody substances specific for the growth factors or their ligands. The invention further contemplates treating lymphangioamas, lymphangiosarcomas, and metastatic tumors, which exhibit VEGFR-3 expressing vascular endothelial cells or VEGFR-3 expressing lymphatic endothelial cells. In one embodiment, administration of a composition that inhibits the interaction of VEGFR-3 with its ligand diminishes or abolishes lymphangiogenesis and retards the spread of cancerous cells. In an additional embodiment, administration of a composition that stimulates the interaction of VEGFR-3 with its ligand enhances lymphangiogenesis and speeds wound healing.

Further contemplated is a method of modulating growth, migration, or proliferation of cells in a mammalian organism, comprising steps of:

(a) identifying a mammalian organism having cells that express a neuropilin receptor; and

(b) administering to said mammalian organism a composition, said composition comprising a bispecific antibody specific for the neuropilin receptor and for a VEGF-C polypeptide, wherein the composition is administered in an amount effective to modulate growth, migration, or proliferation of cells that express the neuropilin receptor in the mammalian organism. In an alternative embodiment, the bispecific antibody is specific for the neuropilin receptor and for a VEGFR-3 polypeptide.

In one embodiment, the invention provides a bispecific antibody which specifically binds a neuropilin receptor and a VEGF-C polypeptide. Alternatively, the invention provides a bispecific antibody which specifically binds to the neuropilin receptor and a VEGFR-3 polypeptide.

In another embodiment, the invention can also be used to inhibit neural degeneration in the central nervous system. Development of scars surrounding neuronal injury in either the peripheral and more specifically the central nervous system has been associated with constitutive expression of the semaphorin ligands. Also, upregulation of Sema3F, a primary ligand for the neuropilin-2 receptor, has been detected in the brains of Alzheimer's patients. The present invention provides for a means to alter the semaphorin-neuropilin interactions using VEGF-C compositions that specifically interfere with semaphorin activity in the nervous system.

For example, the invention provides for a method of modulating aberrant growth, or neuronal scarring in a mammalian organism, comprising a step of:

(a) identifying a mammalian organism having neuronal cells that express a neuropilin receptor; and

(b) administering to said mammalian organism a composition, said composition comprising a VEGF-C polypeptide or fragment thereof that binds to the neuropilin receptor;

wherein the composition is administered in an amount effective to reduce neuronal scarring in cells that express neuropilin in the mammalian organism.

Other conditions to treat include inflammatory diseases (e.g., Rheumatoid arthritis, chronic wounds and atherosclerosis).

Similarly, the invention provides a polypeptide comprising a fragment of VEGF-C that binds to a neuropilin receptor, for use in the manufacture of a medicament for the treatment of diseases characterized by aberrant growth, migration, or proliferation of cells that express a neuropilin receptor.

Likewise, the invention provides a polypeptide comprising a fragment of a neuropilin that binds to a VEGF-C, for use in the manufacture of a medicament for the treatment of diseases characterized by aberrant growth, migration, or proliferation of cells that express a neuropilin receptor. Soluble forms of the neuropilin, lacking the transmembrane domain, are preferred. The invention also provides for a polypeptide comprising a fragment of a neuropilin receptor that binds to a VEGFR-3 polypeptide, for use in the manufacture of a medicament for the treatment of diseases characterized by aberrant growth, migration, or proliferation of cells that express a VEGFR-3 polypeptide.

With respect to aspects of the invention that involve administration of protein agents to mammals, a related aspect of the invention comprises gene therapy whereby a gene encoding the protein of interest is administered in a manner to effect expression of the protein of interest in the animal. For example, the gene of interest is attached to a suitable promoter to promote expression of the protein in the target cell of interest, and is delivered in any gene therapy vector capable of delivering the gene to the cell, including adenovirus vectors, adeno-associated virus vectors, liposomes, naked DNA transfer, and others.

The evidence described herein that VEGF-C functions as a neurotrophic and neuroprotective growth factor supports new therapeutic strategies to treat disorders in which neuronal loss or functional deficiency is a problem. Additionally, the invention provides methods of using “VEGF-C inhibitors” to inhibit neuroblastoma or other tumors of neural origin.

In one embodiment, the invention provides a method of promoting recruitment, proliferation, differentiation, migration or survival of neuronal cells or neuronal precursor cells in a mammalian subject comprising administering to the subject a composition comprising a vascular endothelial growth factor C (VEGF-C) product or a vascular endothelial growth factor D (VEGF-D) product. The term “recruitment” refers to the ability to cause mobilization (e.g. migration) of a cell type, such as mobilization of neuronal cells and neuronal precursor cells to a site of neuropathology). The term “proliferation” refers to mitotic reproduction. The term “differentiation” refers to the process by which the pluripotent and other, non-terminally differentiating neuronal precursor cells develop into other cell types. Differentiation may involve a number of stages between pluripotency and fully differentiated cell types. The term “survival” refers to the ability of the neurons or precursor cells to maintain metabolic and other cellular functions.

The term “VEGF-C products” useful in the invention includes any full-length (prepro-) VEGF-C polypeptide; fragments thereof that retain at least one biological activity of a VEGF-C polypeptide, such as binding to a VEGF-C receptor; VEGF-C polynucleotides and fragments thereof that encode and can be used to express a VEGF-C polypeptide; vectors (especially expression vectors and gene therapy vectors) that comprises such polynucleotides; and recombinant cells that express VEGF-C polypeptides.

VEGF-C polypeptides occur naturally as prepro-peptides that undergo proteolytic processing of signal-peptide and C-terminal pro-peptides before secretion into the surrounding milieu. Further proteolytic processing to cleave an N-terminal pro-peptide releases a fully processed from of VEGF-C. “VEGF-C product” includes a prepro-VEGF-C polypeptide, the intermediate and final cleavage products of prepro-VEGF-C, VEGF-C, ΔNΔC, VEGF-C ΔC156. VEGF-C C156S, VEGF-C ΔNΔC C156S, a chimeric heparin-binding VEGF-C, or a fragment of pre-pro VEGF-C that binds a VEGF-C receptor selected from the group consisting of VEGFR-2. VEGFR-3, neuropilin-1 and neuropilin-2. Preferably, the VEGF-C polypeptide comprises the amino acid sequence set forth in SEQ ID NO: 24 or comprises a fragment thereof that binds to VEGFR-2 or VEGFR-3 and stimulates VEGFR-2 or VEGFR-3 phosphorylation in cells that express one or both of these receptors. Experimental evidence indicates that certain VEGF-C polypeptides do not bind both neuropilins and VEGFR. For example, VEGF-C ΔNΔC does not bind neuropilin receptors but does bind VEGFR-3. It is expected, however, that VEGF-C polypeptides lacking neuropilin binding properties, when acting through VEGF receptors, would have neurotrophic properties similar to those neurotrophic affects mediated through VEGF-C/VEGFR interactions.

Exemplary heparin binding VEGF-C polypeptides are described in co-owned, co-pending U.S. Provisional Patent Application No. 60/478,390 (incorporated herein by reference). Exemplary chimeric heparin binding VEGF-C polypeptides comprise the VEGF homology domain (VHD) of VEGF-C fused to heparin-binding domain of VEGF, such as exons 6-8 (CA89) or exons 7-8 (CA65) encoded sequences, which both contain the neuropilin binding region, VEGF exon 7. In expression studies, CA65 is secreted and released into the supernatant, but CA89 is not released into the supernatant unless heparin is included in the culture medium, indicating that it apparently binds to cell surface heparin sulfates similar to what has been described for VEGF189.

In one embodiment the VEGF-C product comprises a fragment of human prepro-VEGF-C that contains amino acids 103-227 of SEQ. ID NO: 24. In another embodiment, the VEGF-C product comprises amino acids 32-227 of the human prepro-VEGF-C sequence of SEQ. ID NO.: 24. In an additional embodiment, polypeptides having an amino acid sequence comprising a continuous portion of SEQ ID NO: 24, the continuous portion having, as its amino terminus, an amino acid selected from the group consisting of positions 32-111 of SEQ ID NO: 2, and having, as its carboxyl terminus, an amino acid selected from the group consisting of positions 228-419 of SEQ ID NO: 24 are contemplated. As explained elsewhere herein in greater detail, VEGF-C biological activities increase upon processing of both an amino-terminal and carboxyl-terminal pro-peptide. Thus, an amino terminus selected from the group consisting of positions 102-131 of SEQ ID NO: 24 or positions 103-111 of SEQ ID NO: 24 are contemplated. Likewise, a carboxyl terminus selected from the group consisting of positions 215-227 of SEQ ID NO: 2 is contemplated.

While it will be apparent that the method will likely give its best results if the functional portion of the chosen VEGF-C is identical in amino acid sequence to the corresponding portion of the native VEGF-C, it will be apparent that the invention can still be practiced if variations have been introduced in the VEGF-C sequence that do not eliminate its receptor binding properties. The term “VEGF-C product” also is intended to encompass polypeptides encoded by allelic variants of the human VEGF-C characterized by the sequences set forth in SEQ ID NOs: 23 and 24. Use of variant sequences with at least 90%, 95%, 96%, 97%, 98%, or 99% amino acid identity also is specifically contemplated.

In another variation, the VEGF-C product comprises a polynucleotide that encodes a VEGF-C polypeptide product and that can be expressed in a cell. For example, the VEGF-C product comprises a polynucleotide selected from the group consisting of: (a) a polynucleotide comprising a nucleotide sequence that encodes the human VEGF-C amino acid sequence of SEQ ID NO: 24; (b) a polynucleotide comprising a nucleotide sequence at least 90% identical to the nucleotide sequence of SEQ ID NO: 23 encoding a polypeptide that binds VEGFR-3; (c) a polynucleotide comprising a nucleotide sequence that encodes a polypeptide comprising an amino acid sequence at least 90% identical to SEQ ID NO: 24, wherein the polypeptide binds VEGFR-3; (d) a polynucleotide that hybridizes to the complement of SEQ ID NO: 23 under the following stringent conditions and encodes a polypeptide that binds VEGFR-3: 2×SSC/0.1% SDS twice at RT, 1×SSC/0.1% SDS 15 min at 55° C., 0.1×SSC/0.1% SDS 15 min at 55° C.; and (e) fragments of (a)-(d) that encoded a polypeptide that binds VEGFR-3. Conditions of equivalent stringency can be achieved through variation of temperature and buffer, or salt concentration as described Ausubel et al. (Eds.), Current Protocols in Molecular Biology, John Wiley & Sons (1994), pp. 6.0.3-6.4.10.

Preferred VEGF-C polynucleotides encode VEGF-C polypeptides as described above, including full-length prepro-VEGF-C, intermediate and final cleavage products of VEGF-C, as well as fragments and variants thereof. In one embodiment, the VEGF-C product comprises a polynucleotide that encodes a VEGF-C polypeptide set forth in SEQ ID NO: 24 or fragment thereof that binds VEGFR-2, VEGFR-3, NRP-1 or NRP-2. Polynucleotides preferably include a promoter and/or enhancer to promote expression of the encoded VEGF-C protein in target cells of the recipient organism, as well as a stop codon, a polyadenylation signal sequence, and other sequences to facilitate expression.

The promoter can be either a viral promoter or a cell-specific promoter. In one embodiment, the VEGF-C product comprises an expression vector containing the VEGF-C-encoding polynucleotide. In another embodiment, the method provides a VEGF-C product wherein the VEGF-C product comprises a viral vector containing the polynucleotide, such as replication-deficient adenoviral and adeno-associated viral vectors, and hybrids thereof. It is further contemplated that the composition that comprises the VEGF-C product further comprises a pharmaceutically acceptable carrier.

As described below in greater detail, the growth factor VEGF-D shares amino acid sequence similarity to VEGF-C, is known to undergo similar proteolytic processing from a prepro-VEGF-D form into smaller, secreted growth factor forms, and is known to share two VEGF receptors with VEGF-C, namely, VEGFR-3 and VEGFR-2. Due to these and other similarities, it is expected that VEGF-D polypeptides acting through VEGF receptors would have neurotrophic properties similar to those neurotrophic affects mediated through VEGF-C/VEGFR interactions.

Accordingly, as another aspect of the invention, practice of the above-described method of stimulating neural stem cells (and other methods described in the ensuing paragraphs) is contemplated wherein a VEGF-D product is administered in lieu of (or in addition to) a VEGF-C product.

Similar to the VEGF-C product, the term “VEGF-D product” includes a prepro-VEGF-D polypeptide and fragments thereof that bind and stimulate a VEGF-D receptor, as VEGF-D polynucleotides and expression containing them, such as replication-deficient adenoviral, adeno-associated viral and lentiviral vectors, and hybrids thereof. A detailed description of the human VEGF-D gene and protein are provided in Achen, et al. Proc. Nat'l Acad. Sci. U.S.A., 95(2): 548-553 (1998); International Patent Publication No. WO 98/07832, published 26 February 1998; and in Genbank Accession No. AJ000185, all incorporated herein by reference. A cDNA and deduced amino acid sequence for human prepro-VEGF-D is set forth herein in SEQ ID NOs: 25 and 26.

The mammalian subject may be human, or any animal model for human medical research, or an animal of importance as livestock or pets. In a preferred variation, the subject has a disease or condition characterized by a need for stimulating neuronal, neural precursor or neural stem cell recruitment, proliferation, or differentiation, and the administration of the VEGF-C product or VEGF-D product improves the animal's state (e.g., by palliating disease symptoms, slowing disease progression, curing the disease, or otherwise improving clinical outcome).

In one variation, the method further comprises a step, prior to the administration, of identifying a subject in need of neuronal cell or neuronal precursor cell recruitment, proliferation, differentiation, migration or survial. The identifying step involves a medical diagnosis to identify a subject that suffers from a disease or condition that would benefit from neural stem cell recruitment, proliferation, or differentiation. This can be performed by motor skills assessment, MRI brain imaging, and other tests commonly used in the art for monitoring neurodegenerative disease and neuropathologies. Diagnosis may optionally include biopsies and/or cell-based in vitro measurement of neuronal damage. For example, in subjects suspected to have Alzheimer's disease, an in vitro assay may measure the levels of amyloid beta protein, a molecule generally associated with Alzheimer's disease, to determine the extent of amyloid plaque formation in the brain; also, in patient's with Alzheimer's or Parkinson's disease, levels of acetylcholine or acetylcholine receptor may be measured (Banerjee et al., Neurobiol Dis. 7:666-72. 2000).

In a preferred embodiment, the subject to be treated and the VEGF-C polypeptide or VEGF-D polypeptide are human.

Another embodiment of the invention provides a method of stimulating neural stem cell proliferation or differentiation, comprising obtaining a biological sample from a mammalian subject, wherein said sample comprises neural stem cells (NSC), and contacting the stem cells with a composition comprising a vascular endothelial growth factor C (VEGF-C) product or vascular endothelial growth factor D (VEGF-D) product. In one aspect, the contacting comprises culturing the stem cells in a culture containing the VEGF-C product or VEGF-D product. In this method, the beneficial effects of the VEGF-C or VEGF-D are imparted to cells from a human or animal subject outside of the body of the human or other animal subject. Such therapy may be desirable to avoid side-effects, or to prepare a cell sample for use in a medical procedure.

The biological sample can be any tissue or fluid sample from which stem cells are found. Blood and bone marrow are practicable sources for the biological sample, as is umbilical cord blood. Neural stem cells are also isolated from the brain, including the hippocampus, olfactory lobe or adult ventricular zone, of adult mammals.

In one aspect, the biological sample is subjected to purification and/or isolation procedures to purify or isolate the stem cells before the contacting step. In a related aspect, the method further comprises a step of purifying and isolating the neural stem cells or neural cells after the contacting step. Likewise, the invention contemplates purified or isolated neural stem cells cultured with VEGF-C or VEGF-D, in order to select those cells that have proliferated or differentiated in response to VEGF-C or VEGF-D treatment. Neural stem cells are induced to differentiate into any neural cells including glia, oligodendrocytes, neurons, or astrocytes. Cells are characterized as multipotent neural progenitor cells based on the ability to propagate over many passages, expression of nestin and Ki-67, proto-neuronal morphology, as well as the ability to differentiate into neurons and glia.

In one embodiment, human subjects are contemplated. In another embodiment, when the subject is human, the cell donor is a close relative, or has a substantially identical human leukocyte antigen (HLA) profile. In one variation, the cells are seeded into a tissue, organ, or artificial matrix ex vivo, and said tissue, organ, or artificial matrix is attached, implanted, or transplanted into the mammalian subject.

Other sources of NSCs include the spinal cord, fetal tissue, retina, and embryo. Neuron specific markers useful in the invention for isolating neural stem cells and differentiated cells include neurofilament protein (NFP), which stain neurons, and glial fibrillary acidic protein (GFAP) which identifies cells of a glial lineage. Other positive neural stem cell markers are selected from the group consisting of: CD9, CD15, CD95, CD3, MHC 1 and β2 microglobulin (see U.S. Patent Publ. No. 20030040023)

Stem cells from the neural retina express the markers previously shown for brain-derived stem cells, GD2 ganglioside, CD15, and the tetraspanins CD9 and CD81, GD2 and CD15 were recently shown to be markers of true neural stem cells, whereas the tetraspanins CD9 and CD81 show less specificity for true stem cells.

In one variation, the method further comprises a step of administering the neuronal stem cells to a mammalian subject after the contacting step. In another embodiment, the method comprises a step of transplanting the neural cells into a different mammalian subject after the contacting step. In a variation of the method, the cells are seeded into a tissue, organ, or artificial matrix ex vivio, and said tissue, organ, or artificial matrix is attached, implanted, or transplanted into a mammalian subject. It is contemplated that the mammalian subject is human.

The neural stem cells may be administered or transplanted into a mammalian subject in a manner appropriate for the disease or condition being treated, e.g. either systemically, or locally at the site of neuropathology, as described in the Detailed Description.

Another embodiment of the invention is a method of inducing neural stem cell proliferation in vitro comprising contacting the neural stem cell with a composition comprising the VEGF-C product or VEGF-D product, wherein the neural stem cell is selected from the group consisting of the neural stem cell line C17.2, purified neural stem cells, HSN-1 cells, fetal pig cells, neural crest cells, bone marrow derived neural stem cells, hNT cells and a human neuronal progenitor cell line.

In one variation, the contacting step comprises culturing the stem cells in a culture containing the VEGF-C product. For example, 1-100 μg protein/mL growth medium is employed. In still another variation, the contacting comprises transforming or transfecting the stem cells with a VEGF-C transgene.

Optionally, the method further comprises a step of administering the stem cells to a mammalian subject after the contacting step. In a variation of the method, the cells are seeded into a tissue, organ, or artificial matrix ex vivo, and said tissue, organ, or artificial matrix is attached, implanted, or transplanted into a mammalian subject. It is contemplated that the mammalian subject is human.

It is further contemplated that the methods of the invention are carried out wherein the VEGF-C product or VEGF-D product is administered in conjunction with a neural growth factor. Exemplary neural growth factors include, but are not limited to, interferon gamma, nerve growth factor, epidermal growth factor (EGF), basic fibroblast growth factor (bFGF), neurogenin, brain derived neurotrophic factor (BDNF), thyroid hormone, bone morphogenic proteins (BMPs), leukemia inhibitory factor (LIF), sonic hedgehog, and glial cell line-derived neurotrophic factor (GDNF), vascular endothelial growth factor (VEGF), interleukins, interferons, stem cell factor (SCF), activins, inhibins, chemokines, retinoic acid and ciliary neurotrophic factor (CNTF). In one aspect, the invention contemplates a composition comprising the VEGF-C product and a neural growth factor in a pharmaceutically acceptable diluent or carrier.

Methods of the invention preferably are performed wherein the subject has a disease or condition characterized by aberrant growth of neuronal cells, neuronal scarring and damage or neural degeneration. A disease or medical disorder is considered to be nerve damage if the survival or function of nerve cells and/or their axonal processes is compromised. Such nerve damage occurs as the result of conditions including; physical injury, which causes the degeneration of the axonal processes and/or nerve cell bodies near the site of the injury; ischemia, as a stroke; exposure to neurotoxins, such as the cancer and AIDS chemotherapeutic agents such as cisplatin and dideoxycytidine (ddC), respectively; chronic metabolic diseases, such as diabetes or renal dysfunction; and neurodegenerative diseases such as Parkinson's disease, Alzheimer's disease, and Amyotrophic Lateral Sclerosis (ALS), which cause the degeneration of specific neuronal populations. Conditions involving nerve damage include Parkinson's disease, Alzheimer's disease, Amyotrophic Lateral Sclerosis, stroke, diabetic polyneuropathy, toxic neuropathy, glial-scar, and physical damage to the nervous system such as that caused by physical injury of the brain and spinal cord or crush or cut injuries to the arm and hand or other parts of the body, including temporary or permanent cessation of blood flow to parts of the nervous system, as in stroke.

In one embodiment, the disease or condition being treated is a neurodegenerative disorder, wherein the neurodegenerative disorder is selected from the group consisting of Alzheimer's disease, Parkinson's disease, Huntington's disease, motor neuron disease, Amyotrophic Lateral Sclerosis (ALS), dementia and cerebral palsy. In another embodiment, the disease or condition is selected from the group consisting of neural trauma or neural injury. Methods of the invention also can be performed to treat or ameliorate the effects of neural trauma or injury, such as injury related to stroke, spinal cord injury, post-operative injury, brain ischemia and other traumas.

The invention can be used to treat one or more adverse consequences of central nervous system injury that arise from a variety of conditions. Thrombus, embolus, and systemic hypotension are among the most common causes of stroke. Other injuries may be caused by hypertension, hypertensive cerebral vascular disease, rupture of an aneurysm, an angioma, blood dyscrasia, cardiac failure, cardiac arrest, cardiogenic shock, kidney failure, septic shock, head trauma, spinal cord trauma, seizure, bleeding from a tumor, or other loss of blood volume or pressure. These injuries lead to disruption of physiologic function, subsequent death of neurons, and necrosis (infarction) of the affected areas. The term “stroke” connotes the resulting sudden and dramatic neurologic deficits associated with any of the foregoing injuries.

The terms “ischemia” or “ischemic episode,” as used herein, means any circumstance that results in a deficient supply of blood to a tissue. Thus, a central nervous system ischemic episode results from an insufficiency or interruption in the blood supply to any locus of the brain such as, but not limited to, a locus of the cerebrum, cerebellum or brain stem. The spinal cord, which is also a part of the central nervous system, is equally susceptible to ischemia resulting from diminished blood flow. An ischemic episode may be caused by a constriction or obstruction of a blood vessel, as occurs in the case of a thrombus or embolus. Alternatively, the ischemic episode may result from any form of compromised cardiac function, including cardiac arrest, as described above. Where the deficiency is sufficiently severe and prolonged, it can lead to disruption of physiologic function, subsequent death of neurons, and necrosis (infarction) of the affected areas. The extent and type of neurologic abnormality resulting from the injury depend on the location and size of the infarct or the focus of ischemia. Where the ischemia is associated with a stroke, it can be either global or focal in extent.

It is expected that the invention will also be useful for treating traumatic injuries to the central nervous system that are caused by mechanical forces, such as a blow to the head. Trauma can involve a tissue insult selected from abrasion, incision, contusion, puncture, compression, etc., such as can arise from traumatic contact of a foreign object with any locus of or appurtenant to the mammalian head, neck or vertebral column. Other forms of traumatic injury can arise from constriction or compression of mammalian CNS tissue by an inappropriate accumulation of fluid (e.g. a blockade or dysfunction of normal cerebrospinal fluid or vitreous humour fluid production, turnover or volume regulation, or a subdural or intracranial hematoma or edema). Similarly, traumatic constriction or compression can arise from the presence of a mass of abnormal tissue, such as a metastatic or primary tumor.

It is further contemplated that methods of the invention can be practiced by co-administering a VEGF-C product or VEGF-D product with a neurotherapeutic agent. By “neurotherapeutic agent” is meant an agent used in the treatment of neurodegenerative diseases or to treat neural trauma and neural injury. Exemplary neurotherapeutic agents include tacrine (Cognex), donepezil (Aricept), rivastigmine (Exelon), galantamine (Reminyl), and cholinesterase inhibitors and anti-inflammatory drugs, which are useful in the treatment of Alzheimer's disease as well as other neurodegenerative diseases.

Additional neurotherapeutic agents include anti-cholinergics, dopamine agonists, catechol-O-methyl-transterases (COMTs), amantadine (Symmetrel), Sinemet®, Selegiline, carbidopa, ropinirole (Requip). coenzyme Q10, Pramipexole (Mirapex) and levodopa (L-dopa), which are useful in the treatment of Parkinson's disease as well as other neurodegenerative diseases. More therapeutics are set out in the Detailed Description.

It is also contemplated that inhibition of VEGF-C activity is useful therapy for pathologies characterized by hyperproliferation of neuronal cells. Inhibition of VEGF-C in neuronal stem cell development can decrease the proliferation of neuronal cells that cause neuroblastoma (e.g. sympathetic ganglia) and other neural derived tumors, thereby decreasing the cancer's progression. The most common brain tumors are gliomas, which begin in the glial tissue. Astrocytomas, which arise from small, star-shaped cells called astrocytes, most often arise in the adult cerebrum. A grade III astrocytoma is sometimes called anaplastic astrocytoma. A grade IV astrocytoma is usually called glioblastoma multiforme. Brain stem gliomas occur in the lowest, stem-like part of the brain. The brain stem controls many vital functions. Most brain stem gliomas are high-grade astrocytomas. Ependymomas usually develop in the lining of the ventricles. They may also occur in the spinal cord. Oligodendrogliomas arise in the cells that produce myelin, the fatty covering that protects nerves. These tumors usually arise in the cerebrum. They grow slowly and usually do not spread into surrounding brain tissue. Medulloblastomas develop from primitive nerve cells that normally do not remain in the body after birth. For this reason, medulloblastomas are sometimes called primitive neuroectodermal tumors (PNET). Most medulloblastomas arise in the cerebellum; however, they may occur in other areas as well. Meningiomas grow from the meninges. They are usually benign. Because these tumors grow very slowly, the brain may be able to adjust to their presence; meningiomas often grow quite large before they cause symptoms. They occur most often in women between 30 and 50 years of age. Schwannomas are benign tumors that begin in Schwann cells, which produce the myelin that protects the acoustic nerve. Acoustic neuromas are a type of schwannoma. Craniopharyngiomas develop in the region of the pituitary gland near the hypothalamus. They are usually benign; however, they are sometimes considered malignant because they can press on or damage the hypothalamus and affect vital functions. Germ cell tumors arise from primitive (developing) sex cells, or germ cells. The most frequent type of germ cell tumor in the brain is the germinoma. Pineal region tumors occur in or around the pineal gland. The tumor can be slow growing pineocytoma or fast growing (pineoblastoma). The pineal region is very difficult to reach, and these tumors often cannot be removed. Treatment for a brain tumor depends on a number of factors. Among these are the type, location, and size of the tumor, as well as the patient's age and general health. Normally brain tumors are treated with surgery, radiation therapy, and chemotherapy. In one aspect, the invention provides a method of inhibiting growth and progression of neuroblastoma and neural tumors comprising administering to a subject having a neuroblastoma or neuronal tumor a composition comprising a VEGF-C or VEGF-D inhibitor.

The VEGF-C inhibitor can be any molecule that acts with specificity to reduce VEGF-C mitogenic activity, e.g., by blocking VEGF-C binding to any one of its receptors, VEGFR-2, VEGFR-3, NRP-1 or NRP-2, or by reducing expression of VEGF-C. The VEGF-C inhibitor administered can be a polypeptide comprising a soluble VEGFR-2 polypeptide fragment that binds to VEGF-C protein, a soluble VEGFR-3 polypeptide fragment that binds to VEGF-C protein, a soluble NRP-1 polypeptide fragment that binds to VEGF-C protein, a soluble NRP-2 polypeptide fragment that binds to VEGF-C protein. VEGF-C anti-sense polynucleotides or short-interfering RNA (siRNA), an anti-VEGF-C antibody, a polypeptide comprising an antigen binding fragment of an anti-VEGF-C antibody and any small molecule inhibitor of VEGF-C. VEGF-D inhibitors similar to the above-mentioned VEGF-C inhibitors are contemplated for the invention.

In one aspect, the VEGF-C inhibitor comprises a soluble VEGFR-2. VEGFR-3, NRP-1 or NRP-2 polypeptide fragment comprising an extracellular domain fragment of mammalian VEGFR-2, an extracellular domain fragment of VEGFR-3, an extracellular domain fragment of NRP-1 or an extracellular domain fragment of NRP-2, wherein said fragment binds to VEGF-C protein. Preferably, the VEGFR-2, VEGFR-3, NRP-1 or NRP-2 fragment is human. In one variation, the VEGFR-3 extracellular domain fragment comprises immunoglobulin domains one through three of VEGFR-3. In another embodiment, the extracellular domain fragment contemplated by the invention comprises amino acids 33 to 324 of human VEGFR-3 set out in SEQ ID NO: 32. In an alternate embodiment, the soluble VEGFR-2, VEGFR-3, NRP-1 or NRP-2 fragment is linked to an immunoglobulin Fc domain.

In one embodiment, the VEGF-C inhibitor comprises a polypeptide comprising an amino acid sequence comprising at least 90%, 95%, 96%, 97%, 98%, or 99% identical to amino acids comprising the extracellular fragment of human VEGFR-2 (SEQ ID NO: 30) that maintains VEGF-C binding activity, an amino acid sequence comprising at least 90%, 95%, 96%, 97%, 98%, or 99% amino acid identity to amino acids comprising the extracellular fragment of human VEGFR-3 (SEQ ID NO: 32) that maintains VEGF-C binding activity, an amino acid sequence comprising at least 90%, 95%, 96%, 97%, 98%, or 99% amino acid identity to amino acids comprising the extracellular fragment of human NRP-1 (SEQ ID NO: 2) that maintains VEGF-C binding activity, or an amino acid sequence comprising at least 90%, 95%, 96%, 97%, 98%, or 99% amino acid identity to amino acids comprising the extracellular fragment of human NRP-2 polypeptide (SEQ ID NO: 4) that maintains VEGF-C binding activity.

In an additional embodiment, the VEGF-C inhibitor composition comprises a polypeptide encoded by a polynucleotide that hybridizes to the complement of a polynucleotide encoding amino acids 33 to 324 of SEQ. ID NO.: 32, under either moderate or highly stringent conditions. Exemplary moderately stringent conditions of hybridization are hybridization in 0.5 M NaHPO4, 7% sodium dodecyl sulfate (SDS), 1 mM EDTA at 65° C. and washing in 0.2×SSC/0.1% SDS at 42° C. Exemplary highly stringent hybridization conditions are: 0.5 M NaHPO₄, 7% sodium dodecyl sulfate (SDS), 1 mM EDTA at 65° C. and washing in 0.1×SSC/0.1% SDS at 68° C. It is understood in the art that conditions of equivalent stringency can be achieved through variation of temperature and buffer, or salt concentration as described Ausubel et al. (Eds.), Current Protocols in Molecular Biology, John Wiley & Sons (1994), pp. 6.0.3-6.4.10.

VEGF-C antisense nucleic acid molecules for use in the method comprise a sequence complementary to any integer number of nucleotides from the target sequence, from about 10 to 500, preferably an integer number from 10 to 50. In exemplary embodiments, a VEGF-C antisense molecule comprises a complementary sequence at least about 10, 25, 50, 100, 250 or 500 nucleotides in length or complementary to an entire VEGF-C coding strand. More specifically, antisense molecules of 10, 15, 20, 25, 30, 35, 40, 45, or 50 nucleotides in length are contemplated.

The siRNAs contemplated for use in the invention provide both a sense and antisense coding strand of the VEGF-C mRNA. siRNAs are typically 30 nucleotides or less in length, and more preferably 21- to 23-nucleotides, with characteristic 2- to 3-nucleotide 3′-overhanging ends, which are generated by ribonuclease III cleavage from longer dsRNAs.

The present invention also provides a composition comprising a VEGF-C product or a VEGF-D product and a neural growth factor in a pharmaceutically acceptable diluent or carrier. The invention further contemplates a composition comprising a VEGF-C product or a VEGF-D product and a neurotherapeutic agent in a pharmaceutically acceptable diluent or carrier.

Additional features and variations of the invention will be apparent to those skilled in the art from the entirety of this application, and all such features are intended as aspects of the invention.

Likewise, features of the invention described herein can be re-combined into additional embodiments that also are intended as aspects of the invention, irrespective of whether the combination of features is specifically mentioned above as an aspect or embodiment of the invention. Also, only such limitations which are described herein as critical to the invention should be viewed as such; variations of the invention lacking limitations which have not been described herein as critical are intended as aspects of the invention.

In addition to the foregoing, the invention includes, as an additional aspect, all embodiments of the invention narrower in scope in any way than the variations specifically mentioned above. Although the applicant(s) invented the full scope of the claims appended hereto, the claims appended hereto are not intended to encompass within their scope the prior art work of others. Therefore, in the event that statutory prior art within the scope of a claim is brought to the attention of the applicants by a Patent Office or other entity or individual, the applicant(s) reserve the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention.

### BRIEF DESCRIPTION OF THE DRAWINGS

FIG. 1 depicts the construction of the neuropilin-2 IgG fusion protein a17 and a22 expression vectors.

### DETAILED DESCRIPTION OF THE INVENTION

The present invention is based, in part, on the discovery of novel interaction between proteins that have previously been characterized in the literature, but whose interactions were not previously appreciated, and whose biological effects were not previously appreciated. A number of the molecules are explicitly set forth with annotations to the Genbank database or to a Sequence Listing appended hereto, but it will be appreciated that sequences for species homologous (“orthologs”) are also easily retrieved from databases and/or isolated from natural sources. Thus, the following table and description should be considered exemplary and not limiting.

A. Molecules of Interest to the Present Invention.

| Molecule | Genbank Accession #* | SEQ ID NO. |
|---|---|---|
| Neuropilin-1 | NM003873 | 1 and 2 |
| Soluble Neuropilin-1, s11 | AF280547 | |
| Neuropilin-2 [a(17)] | NM003872 | 3 and 4 |
| a(0) | AF022859 | |
| a(17) | AF022860 | |
| b(0) | AF280544 | |
| b(5) | AF280545 | |
| Soluble Neuropilin-2, s9 | AF280546 | |
| Murine neuropilin-1 | D50086 | 5 and 6 |
| Murine neuropilin-2 | | |
| a(0) | AF022854 | |
| a(5) | AF022861 | |
| a(17) | AF022855 | 7 and 8 |
| a(22) | AF022856 | |
| b(0) | AF022857 | |
| b(5) | AF022858 | |
| Semaphorin 3A | NM006080 | 9 and 10 |
| Semaphorin 3B | NM004636 | 11 and 12 |
| Semaphorin 3C | NM006379 | 13 and 14 |
| Semaphorin 3E | NM012431 | 15 and 16 |
| Semaphorin 3F | NM004186 | 17 and 18 |
| VEGF-A | Q16889 | 19 and 20 |
| VEGF165 | M32977 | |
| VEGF-B | U48801 | 21 and 22 |
| VEGF-C | X94216 | 23 and 24 |
| VEGF-D | AJ000185 | 25 and 26 |
| VEGF-E | S67522 | |
| PlGF | NM002632 | 27 and 28 |
| VEGFR-1 | X51602 | |
| VEGFR-2 | L04947 | 29 and 30 |
| VEGFR-3 | X68203 | 31 and 32 |
| Plexin-A1 | X87832 | |
| Plexin-A2 | NM025179 | |
| PDGF-A, -B, -C | NM002607; NM002608; | |
| | NM016205 | |
| PDGFR-A, -B | NM006206; NM002609 | |
| Prox-1 | NM002763 | 37 and 38 |

*All Sequences of Human origin unless otherwise noted.

The Neuropilin Family

The neuropilin-1 and neuropilin-2 genes span over 120 and 112 kb, respectively, and are comprised of 17 exons, five of which are identical in size in both genes, suggesting genetic duplication of these genes (Rossignol et al, Genomics 70:211-22. 2000). Several splice variants of the neuropilins have been isolated to date, the functional significance of which is currently under investigation.

Isoforms of NRP-2, designated NRP2a and NRP2b, were first isolated from the mouse genome (Chen et al., Neuron 19:547-59. 1997). In mouse, NRP2a isoforms contain insertions of 0, 5, 17, or 22 (5+17) amino acids after amino acid 809 of NRP-2 and are named NRP2a(0) (Genbank Accession No. AF022854)(SEQ ID NO. 7 and 8), NRP2a(5) (Genbank Accession No. AF022861), NRP2a(17) (Genbank Accession No. AF022855), and NRP2a(22)(Genbank Accession No. AF022856), respectively. Only two human NRP2a isoforms homologous to the mouse variants NRP2a(17) (Genbank Accession No. AF022860) (SEQ ID NO. 3 and 4) and NRP2a(22), have been elucidated. The human a(22) isoform contains a five amino acid insertion, sequence GENFK, after amino acid 808 in NRP2a(17). Tissue analysis of brain, heart, lung, kidney liver and placenta shows that the a(17) isoform is more abundant in all of these sites.

The human NRP2b isoforms appear to express an additional exon, designated exon 16b, not present in either NRP2a or NRP-1. Two human NRP2b isoforms homologous to mouse NRP2b(0) (Genbank Accession No. AF022857) and NRP2b(5) (Genbank Accession No. AF022858) have been identified which contain either a 0 or 5 amino acid insert (GENFK) after amino acid 808 in NRP2b(0) (Rossignol et al., Genomics 70:211-22. 2000). Tissue distribution analysis demonstrates a higher expression of human NRP2b(0) (Genbank Accession No. AF280544) over NRP2b(5) (Genbank Accession No. AF280545) in adult brain, heart, lung, kidney, liver, and placenta. The NRP2a and NRP2b isoforms demonstrate divergence in their C terminal end, after amino acid 808 of NRP2 which is in the linker region between the c domain and the transmembrane domain. This differential splicing may lead to the difference seen in tissue expression of the two isoforms, where NRP2a is expressed more abundantly in the placenta, liver, and lung with only detectable levels of NRP2b, while NRP2b is found in skeletal muscle where NRP2a expression is low. Both isoforms are expressed in heart and small intestine.

In addition to genetic isoforms of the neuropilins, truncated soluble forms of the proteins have also been cloned (Gagnon et al, Proc. Natl. Acad. Sci USA 97:2573-78 2000; Rossignol et al, Genomics 70:211-22.2000). Naturally occurring truncated forms of the NRP-1 protein, s11NRP1 (Genbank Accession No. AF280547) and s12NRP1, have been cloned, that encode 704 and 644 amino acid neuropilin-1, respectively, and contain the a and b domains but not the c domain. The s12NRP1 variant is generated by pre-mRNA processing in intron 12. The s11NRP1 truncation occurs after amino acid 621 and lacks the 20 amino acids encoded by exon 12, but contains coding sequence found within intron 11 that gives it 83 novel amino acids at the C-terminus. This intron derived sequence does not contain any homology to known proteins.

A natural, soluble form of NRP-2 has also been identified which encodes a 555 amino acid protein containing the a domains, b1 domain, and part of the b2 domain, lacking the last 48 amino acids of this region. The truncation occurs after amino acid 547 within intron 9, thus the protein has been named s9NRP2 (Genbank Accession No. AF2805446), and adds 8 novel amino acids derived from the intron cleavage (VGCSVWRPL) at the C-terminus. Gagnon et al (Proc. Natl. Acad. Sci USA 97:2573-78. 2000) report that soluble neuropilin-1 isoform s12NRP1 is capable of binding VEGF165 equivalent to the full length protein, but acts as an antagonist of VEGF165 binding, inhibiting VEGF165 activity and showing anti-tumor properties in a rat prostate carcinoma model.

The PDGF/VEGF Family

The PDGF/VEGF family of growth factors includes at least the following members: PDGF-A (see e.g., GenBank Acc. No. X06374), PDGF-B (see e.g., GenBank Acc. No. MI 2783), VEGF (see e.g., GenBank Acc. No. Q16889 referred to herein for clarity as VEGF-A or by particular isoform), PIGF (see e.g., GenBank Acc. No. X54936 placental growth factor), VEGF-B (see e.g., GenBank Acc. No. U48801; also known as VEGF-related factor (VRF)), VEGF-C (see e.g., GenBank Acc. No. X94216; also known as VEGF related protein (VRP or VEGF-2)), VEGF-D (also known as c-fos-induced growth factor (FIGF); see e.g., Genbank Acc. No. AJ000185), VEGF-E (also known as NZ7 VEGF or OV NZ7; see e.g., GenBank Acc. No. S67522), NZ2 VEGF (also known as OV NZ2; see e.g., GenBank Acc. No. S67520), D1701 VEGF-like protein (see e.g., GenBank Acc. No. AF106020; Meyer et al., EMBO J 18:363-374), and NZ10 VEGF-like protein (described in International Patent Application PCT/US99/25869) [Stacker and Achen, Growth Factors 17:1-11 (1999); Neufeld et al., FASEB J 13:9-22 (1999); Ferrara, J Mol Med 77:527-543 (1999)]. The PDGF/VEGF family proteins are predominantly secreted glycoproteins that form either disulfide-linked or non-covalently bound homo- or heterodimers whose subunits are arranged in an anti-parallel manner [Stacker and Achen, Growth Factors 17:1-11 (1999); Muller et al., Structure 5:1325-1338 (1997)].

The VEGF subfamily is composed of PDGF/VEGF members which share a VEGF homology domain (VHD) characterized by the sequence: C-X(22-24)-P-[PSR]-C-V-X(3)-R-C-[GSTA]-G-C-C-X(6)-C-X(32-41)-C.

VEGF-A was originally purified from several sources on the basis of its mitogenic activity toward endothelial cells, and also by its ability to induce microvascular permeability, hence it is also called vascular permeability factor (VPF). VEGF-A has subsequently been shown to induce a number of biological processes including the mobilization of intracellular calcium, the induction of plasminogen activator and plasminogen activator inhibitor-1 synthesis, promotion of monocyte migration in vitro, induction of anti-apoptotic protein expression in human endothelial cells, induction of fenestrations in endothelial cells, promotion of cell adhesion molecule expression in endothelial cells and induction of nitric oxide mediated vasodilation and hypotension [Ferrara, J Mol Med 77: 527-543 (1999); Neufeld et al., FASEB J 13: 9-22 (1999); Zachary, Intl J Biochem Cell Bio 30: 1169-1174 (1998)].

VEGF-A is a secreted, disulfide-linked homodimeric glycoprotein composed of 23 kD subunits. Five human VEGF-A isoforms of 121, 145, 165, 189 or 206 amino acids in length (VEGF₁₂₁₋₂₀₆), encoded by distinct mRNA splice variants, have been described, all of which are capable of stimulating mitogenesis in endothelial cells. However, each isoform differs in biological activity, receptor specificity, and affinity for cell surface- and extracellular matrix-associated heparin-sulfate proteoglycans, which behave as low affinity receptors for VEGF-A. VEGF₁₂₁ does not bind to either heparin or heparin-sulfate; VEGF₁₄₅ and VEGF₁₆₅ (GenBank Acc. No. M32977) are both capable of binding to heparin; and VEGF₁₈₉ and VEGF₂₀₆ show the strongest affinity for heparin and heparin-sulfates. VEGF₁₂₁, VEGF₁₄₅, and VEGF₁₆₅ are secreted in a soluble form, although most of VEGF₁₆₅ is confined to cell surface and extracellular matrix proteoglycans, whereas VEGF189 and VEGF₂₀₆ remain associated with extracellular matrix. Both VEGF₁₈₉ and VEGF₂₀₆ can be released by treatment with heparin or heparinase, indicating that these isoforms are bound to extracellular matrix via proteoglycans. Cell-bound VEGF₁₈₉ can also be cleaved by proteases such as plasmin, resulting in release of an active soluble VEGF₁₁₀. Most tissues that express VEGF are observed to express several VEGF isoforms simultaneously, although VEGF₁₂₁ and VEGF₁₆₅ are the predominant forms, whereas VEGF₂₀₆ is rarely detected [Ferrara, J Mol Med 77:527-543 (1999)]. VEGF₁₄₅ differs in that it is primarily expressed in cells derived from reproductive organs [Neufeld et al., FASEB J 13:9-22 (1999)].

The pattern of VEGF-A expression suggests its involvement in the development and maintenance of the normal vascular system, and in angiogenesis associated with tumor growth and other pathological conditions such as rheumatoid arthritis. VEGF-A is expressed in embryonic tissues associated with the developing vascular system, and is secreted by numerous tumor cell lines. Analysis of mice in which VEGF-A was knocked out by targeted gene disruption indicate that VEGF-A is critical for survival, and that the development of the cardiovascular system is highly sensitive to VEGF-A concentration gradients. Mice lacking a single copy of VEGF-A die between day 11 and 12 of gestation. These embryos show impaired growth and several developmental abnormalities including defects in the developing cardiovasculature. VEGF-A is also required post-natally for growth, organ development, regulation of growth plate morphogenesis and endochondral bone formation. The requirement for VEGF-A decreases with age, especially after the fourth postnatal week. In mature animals, VEGF-A is required primarily for active angiogenesis in processes such as wound healing and the development of the corpus luteum. [Neufeld et al. FASEB J 13:9-22 (1999); Ferrara, J Mol Med 77:527-543 (1999)]. VEGF-A expression is influenced primarily by hypoxia and a number of hormones and cytokines including epidermal growth factor (EGF), TGF-β, and various interleukins. Regulation occurs transcriptionally and also post-transcriptionally such as by increased mRNA stability [Ferrara, supra]

PIGF, a second member of the VEGF subfamily, is generally a poor stimulator of angiogenesis and endothelial cell proliferation in comparison to VEGF-A, and the in vivo role of PIGF is not well understood. Three isoforms of PIGF produced by alternative mRNA splicing have been described [Hauser et al., Growth Factors 9:259-268 (1993); Maglione et al., Oncogene 8:925-931 (1993)]. PIGF forms both disulfide-linked homodimers and heterodimers with VEGF-A. The PIGF-VEGF-A heterodimers are more effective at inducing endothelial cell proliferation and angiogenesis than PIGF homodimers. PIGF is primarily expressed in the placenta, and is also co-expressed with VEGF-A during early embryogenesis in the trophoblastic giant cells of the parietal yolk sac [Stacker and Achen, Growth Factors 17:1-11 (1999)].

VEGF-B. described in detail in International Patent Publication No. WO 96/26736 and U.S. Pat. Nos. 5,840,693 and 5,607,918, incorporated herein by reference, shares approximately 44% amino acid identity with VEGF-A. Although the biological functions of VEGF-B in vivo remain incompletely understood, it has been shown to have angiogenic properties, and may also be involved in cell adhesion and migration, and in regulating the degradation of extracellular matrix. It is expressed as two isoforms of 167 and 186 amino acid residues generated by alternative splicing. VEGF-B₁₆₇ is associated with the cell surface or extracellular matrix via a heparin-binding domain, whereas VEGF-B₁₈₆ is secreted. Both VEGF-B₁₆₇ and VEGF-B₁₈₆ can form disulfide-linked homodimers or heterodimers with VEGF-A. The association to the cell surface of VEGF₁₆₅-VEGF-B₁₆₇ heterodimers appears to be determined by the VEGF-B component, suggesting that heterodimerization may be important for sequestering VEGF-A. VEGF-B is expressed primarily in embryonic and adult cardiac and skeletal muscle tissues [Joukov et al., J Cell Physiol 173:211-215 (1997); Stacker and Achen, Growth Factors 17:1-11 (1999)]. Mice lacking VEGF-B survive but have smaller hearts, dysfunctional coronary vasculature, and exhibit impaired recovery from cardiac ischemia [Bellomo et al., Circ Res 2000;E29-E35].

A fourth member of the VEGF subfamily, VEGF-C, comprises a VHD that is approximately 30% identical at the amino acid level to VEGF-A. VEGF-C is originally expressed as a larger precursor protein, prepro-VEGF-C, having extensive amino- and carboxy-terminal peptide sequences flanking the VHD, with the C-terminal peptide containing tandemly repeated cysteine residues in a motif typical of Balbiani ring 3 protein. Prepro-VEGF-C undergoes extensive proteolytic maturation involving the successive cleavage of a signal peptide, the C-terminal pro-peptide, and the N-terminal pro-peptide to produce a fully processed mature form (ΔNΔC VEGF-C). Secreted VEGF-C protein comprises a non-covalently-linked homodimer, in which each monomer contains the VHD. The intermediate forms of VEGF-C produced by partial proteolytic processing show increasing affinity for the VEGFR-3 receptor, and the mature protein is also able to bind to the VEGFR-2 receptor. [Joukov et al., EMBO J., 16:(13):3898-3911 (1997).] It has also been demonstrated that a mutant VEGF-C (VEGF-C ΔC₁₅₆), in which a single cysteine at position 156 is either substituted by another amino acid or deleted, loses the ability to bind VEGFR-2 but remains capable of binding and activating VEGFR-3 [U.S. Pat. No. 6,130,071 and International Patent Publication No. WO 98/33917]. Exemplary substitutions at amino acid 156 of SEQ. ID NO: 24 include substitution of a serine residue for the cytsteine at position 156 (VEGF-C C156S). In mouse embryos. VEGF-C mRNA is expressed primarily in the allantois, jugular area, and the metanephros. [Joukov et al., J Cell Physiol 173:211-215 (1997)]. VEGF-C is involved in the regulation of lymphatic angiogenesis: when VEGF-C was overexpressed in the skin of transgenic mice, a hyperplastic lymphatic vessel network was observed, suggesting that VEGF-C induces lymphatic growth [Jeltsch et al., Science, 276:1423-1425 (1997)]. Continued expression of VEGF-C in the adult also indicates a role in maintenance of differentiated lymphatic endothelium [Ferrara, J Mol Med 77:527-543 (1999)]. VEGF-C also shows angiogenic properties: it can stimulate migration of bovine capillary endothelial (BCE) cells in collagen and promote growth of human endothelial cells [see, e.g., U.S. Pat. No. 6,245,530; U.S. Pat. No. 6,221,839; and International Patent Publication No. WO 98/33917, incorporated herein by reference].

The prepro-VEGF-C polypeptide is processed in multiple stages to produce a mature and most active VEGF-C polypeptide of about 21-23 kD (as assessed by SDS-PAGE under reducing conditions). Such processing includes cleavage of a signal peptide (SEQ ID NO: 24, residues 1-31); cleavage of a carboxyl-terminal peptide (corresponding approximately to amino acids 228-419 of SEQ ID NO: 24 to produce a partially-processed form of about 29 kD; and cleavage (apparently extracellularly) of an amino-terminal peptide (corresponding approximately to amino acids 32-102 of SEQ ID NO: 24) to produced a fully-processed mature form of about 21-23 kD. Experimental evidence demonstrates that partially-processed forms of VEGF-C (e.g., the 29 kD form) are able to bind the Flt4 (VEGFR-3) receptor, whereas high affinity binding to VEGFR-2 occurs only with the fully processed forms of VEGF-C. It appears that VEGF-C polypeptides naturally associate as non-disulfide linked dimers.

Moreover, it has been demonstrated that amino acids 103-227 of SEQ ID NO: 24 are not all critical for maintaining VEGF-C functions. A polypeptide consisting of amino acids 112-215 (and lacking residues 103-111 and 216-227) of SEQ ID NO: 24 retains the ability to bind and stimulate VEGF-C receptors, and it is expected that a polypeptide spanning from about residue 131 to about residue 211 will retain VEGF-C biological activity. The cysteine residue at position 156 has been shown to be important for VEGFR-2 binding ability. However. VEGF-C C156 polypeptides (i.e., analogs that lack this cysteine due to deletion or substitution) remain potent activators of VEGFR-3. The cysteine at position 165 of SEQ ID NO: 24 is essential for binding either receptor, whereas analogs lacking the cysteines at positions 83 or 137 compete with native VEGF-C for binding with both receptors and stimulate both receptors. Also contemplated for use in the invention is a chimeric, heparin-binding VEGF-C polypeptide in which a receptor binding VEGF-C sequence is fused to a heparin binding sequence from another source (natural or synthetic). Heparin binding forms of VEGF-C and VEGF-D are described in greater detail in commonly owned, co-filed U.S. Provisional Patent Application No. 60/478,390, incorporated herein by reference. For example, plasmids were constructed encoding chimeric proteins comprised of the signal sequence and the VEGF homology domain (VHD) of VEGF-C (SEQ ID NO: 24), and VEGF exons 6-8 (CA89) or exons 7-8 (CA65) (SEQ ID NO: 20), which encode heparin binding domains. The chimeric polypeptide CA65 was secreted and released into the supernatant, but CA89 was not released into the supernatant unless heparin was included in the culture medium, indicating that it apparently binds to cell surface heparin sulfates similar to what has been described for VEGF189.

VEGF-D is structurally and functionally most closely related to VEGF-C [see U.S. Pat. No. 6,235,713 and International Patent Publ. No. WO 98/07832, incorporated herein by reference]. Like VEGF-C, VEGF-D is initially expressed as a prepro-peptide that undergoes N-terminal and C-terminal proteolytic processing, and forms non-covalently linked dimers. VEGF-D stimulates mitogenic responses in endothelial cells in vitro. During embryogenesis, VEGF-D is expressed in a complex temporal and spatial pattern, and its expression persists in the heart, lung, and skeletal muscles in adults. Isolation of a biologically active fragment of VEGF-D designated VEGF-D ΔNΔC, is described in International Patent Publication No. WO 98/07832, incorporated herein by reference. VEGF-D ΔNΔC consists of amino acid residues 93 to 201 of VEGF-D (SEQ ID NO: 26) optionally linked to the affinity tag peptide FLAG®, or other sequences.

The prepro-VEGF-D polypeptide has a putative signal peptide of 21 amino acids and is apparently proteolytically processed in a manner analogous to the processing of prepro-VEGF-C. A “recombinantly matured” VEGF-D lacking residues 1-92 and 202-354 of SEQ ID NO: 26 retains the ability to activate receptors VEGFR-2 and VEGFR-3, and appears to associate as non-covalently linked dimers. Thus, preferred VEGF-D polynucleotides include those polynucleotides that comprise a nucleotide sequence encoding amino acids 93-201 of SEQ ID NO: 26. The guidance provided above for introducing function-preserving modifications into VEGF-C polypeptides is also suitable for introducing function-preserving modifications into VEGF-D polypeptides. Heparin binding forms of VEGF-D are also contemplated. See U.S. Provisional Patent Application No. 60/478,390, incorporated herein by reference.

Four additional members of the VEGF subfamily have been identified in poxviruses, which infect humans, sheep and goats. The orf virus-encoded VEGF-E and NZ2 VEGF are potent mitogens and permeability enhancing factors. Both show approximately 25% amino acid identity to mammalian VEGF-A, and are expressed as disulfide-linked homodimers. Infection by these viruses is characterized by pustular dermatitis which may involve endothelial cell proliferation and vascular permeability induced by these viral VEGF proteins. [Ferrara, J Mol Med 77:527-543 (1999); Stacker and Achen, Growth Factors 17:1-11 (1999)]. VEGF-like proteins have also been identified from two additional strains of the orf virus, D1701 [GenBank Acc. No. AF106020; described in Meyer et al., EMBO J. 18:363-374 (1999)] and NZ10 [described in International Patent Application PCT/US99/25869, incorporated herein by reference]. These viral VEGF-like proteins have been shown to bind VEGFR-2 present on host endothelium, and this binding is important for development of infection and viral induction of angiogenesis [Meyer et al., supra; International Patent Application PCT/US99/25869].

PDGF/VEGF Receptors

Seven cell surface receptors that interact with PDGF/VEGF family members have been identified. These include PDGFR-α (see e.g., GenBank Acc. No. (NM006206), PDGFR-β (see e.g., GenBank Acc. No. NM002609), VEGFR-1/Flt-1 fms-like tyrosine kinase-1; GenBank Acc. No. X51602; De Vries et al., Science 255:989-991 (1992)); VEGFR-2/KDR/Flk-1 (kinase insert domain containing receptor/fetal liver kinase-1; GenBank Acc. Nos. X59397 (Flk-1) and L04947 (KDR); Terman et al., Biochem Biophys Res Comm 187:1579-1586 (1992); Matthews et al., Proc Natl Acad Sci USA 88:9026-9030 (1991)), VEGFR-3/Flt4 (fms-like tyrosine kinase 4; U.S. Pat. No. 5,776,755 and GenBank Acc. No. X68203 and S66407: Pajusola et al., Oncogene 9:3545-3555 (1994)), neuropilin-1 (Gen Bank Acc. No. NM003873), and neuropilin-2 (Gen Bank Acc. No. NM003872). The two PDGF receptors mediate signaling of PDGFs as described above. VEGF121, VEGF165, VEGF-B, PIGF-1 and PIGF-2 bind VEGF-R1; VEGF121, VEGF145, VEGF165, VEGF-C, VEGF-D, VEGF-E, and NZ2 VEGF bind VEGF-R2; VEGF-C and VEGF-D bind VEGFR-3; VEGF165, VEGF-B, PIGF-2, and NZ2 VEGF bind neuropilin-1; and VEGF165, and VEGF145 bind neuropilin-2.[Neufeld et al. FASEB J 13:9-22 (1999); Stacker and Achen, Growth Factors 17:1-11 (1999); Ortega et al., Fron Biosci 4:141-152 (1999); Zachary, Intl J Biochem Cell Bio 30:1169-1174 (1998); Petrova et al., Exp Cell Res 253:117-130 (1999); Gluzman-Poltorak et al., J. Biol. Chem. 275:18040-45 (2000)].

The PDGF receptors are protein tyrosine kinase receptors (PTKs) that contain five immunoglobulin-like loops in their extracellular domains. VEGFR-1, VEGFR-2, and VEGFR-3 comprise a subgroup of the PDGF subfamily of PTKs, distinguished by the presence of seven Ig domains in their extracellular domain and a split kinase domain in the cytoplasmic region. Both neuropilin-1 and neuropilin-2 are non-PTK VEGF receptors, with short cytoplasmic tails not currently known to possess downstream signaling capacity.

Several of the VEGF receptors are expressed as more than one isoform. A soluble isoform of VEGFR-1 lacking the seventh Ig-like loop, transmembrane domain, and the cytoplasmic region is expressed in human umbilical vein endothelial cells. This VEGFR-1 isoform binds VEGF-A with high affinity and is capable of preventing VEGF-A-induced mitogenic responses [Ferrara et al., J Mol Med 77:527-543 (1999); Zachary, Intl J Biochem Cell Bio 30:1169-1174 (1998)]. A C-terminal truncated from of VEGFR-2 has also been reported [Zachary, supra]. In humans, there are two isoforms of the VEGFR-3 protein which differ in the length of their C-terminal ends. Studies suggest that the longer isoform is responsible for most of the biological properties of VEGFR-3.

The expression of VEGFR-1 occurs mainly in vascular endothelial cells, although some may be present on monocytes and renal mesangial cells [Neufeld et al., FASEB J 13:9-22 (1999)], trophoblast cells (Charnock-Jones, Biol Reprod 51:524-30. 1994), hematopoietic stem cells (Luttun et al. Ann N Y Acad. Sci. 979:80-93. 2002), spermatogenic cells and Leydig cells (Korpelainen et al., J Cell Biol 143:1705-121. 1998) and smooth muscle cells (Ishida et al., J. Cell Physiol. 188:359-68. 2001). High levels of VEGFR-1 mRNA are also detected in adult organs, suggesting that VEGFR-1 has a function in quiescent endothelium of mature vessels not related to cell growth. VEGFR-1−/− mice die in utero between day 8.5 and 9.5. Although endothelial cells developed in these animals, the formation of functional blood vessels was severely impaired, suggesting that VEGFR-1 may be involved in cell-cell or cell-matrix interactions associated with cell migration. Recently, it has been demonstrated that mice expressing a mutated VEGFR-1 in which only the tyrosine kinase domain was missing show normal angiogenesis and survival, suggesting that the signaling capability of VEGFR-1 is not essential. [Neufeld et al., supra; Ferrara, J Mol Med 77:527-543 (1999)].

VEGFR-2 expression is similar to that of VEGFR-1 in that it is broadly expressed in the vascular endothelium, but it is also present in hematopoietic stem cells, megakaryocytes, and retinal progenitor cells [Neufeld et al., supra]. Although the expression pattern of VEGFR-1 and VEGFR-2 overlap extensively, evidence suggests that, in most cell types, VEGFR-2 is the major receptor through which most of the VEGFs exert their biological activities. Examination of mouse embryos deficient in VEGFR-2 further indicate that this receptor is required for both endothelial cell differentiation and the development of hematopoietic cells [Joukov et al., J Cell Physiol. 173:211-215 (1997)].

VEGFR-3 is expressed broadly in endothelial cells during early embryogenesis. During later stages of development, the expression of VEGFR-3 becomes restricted to developing lymphatic vessels [Kaipainen et al., Proc. Natl. Acad. Sci. USA, 92: 3566-3570 (1995)]. In adults, the lymphatic endothelia and some high endothelial venules express VEGFR-3, and increased expression occurs in lymphatic sinuses in metastatic lymph nodes and in lymphangioma. VEGFR-3 is also expressed in a subset of CD34+ hematopoietic cells which may mediate the myelopoietic activity of VEGF-C demonstrated by overexpression studies [WO 98/33917]. Targeted disruption of the VEGFR-3 gene in mouse embryos leads to failure of the remodeling of the primary vascular network, and death after embryonic day 9.5 [Dumont et al., Science, 282: 946-949 (1998)]. These studies suggest an essential role for VEGFR-3 in the development of the embryonic vasculature and also during lymphangiogenesis.

Structural analyses of the VEGF receptors indicate that the VEGF-A binding site on VEGFR-1 and VEGFR-2 is located in the second and third Ig-like loops. Similarly, the VEGF-C and VEGF-D binding sites on VEGFR-2 and VEGFR-3 are also contained within the second Ig-loop [Taipale et al., Curr Top Microbiol Immunol 237:85-96 (1999)]. The second Ig-like loop also confers ligand specificity as shown by domain swapping experiments [Ferrara. J Mol Med 77:527-543 (1999)]. Receptor-ligand studies indicate that dimers formed by the VEGF family proteins are capable of binding two VEGF receptor molecules, thereby dimerizing VEGF receptors. The fourth Ig-like loop on VEGFR-1, and also possibly on VEGFR-2, acts as the receptor dimerization domain that links two receptor molecules upon binding of the receptors to a ligand dimer [Ferrara, J Mol Med 77:527-543 (1999)]. Although the regions of VEGF-A that bind VEGFR-1 and VEGFR-2 overlap to a large extent, studies have revealed two separate domains within VEGF-A that interact with either VEGFR-1 or VEGFR-2, as well as specific amino acid residues within these domains that are critical for ligand-receptor interactions. Mutations within either VEGF receptor-specific domain that specifically prevent binding to one particular VEGF receptor have also been recovered [Neufeld et al., FASEB J 13:9-22 (1999)].

VEGFR-1 and VEGFR-2 are structurally similar, share common ligands (VEGF121 and VEGF165), and exhibit similar expression patterns during development. However, the signals mediated through VEGFR-1 and VEGFR-2 by the same ligand appear to be slightly different. VEGFR-2 has been shown to undergo autophosphorylation in response to VEGF-A, but phosphorylation of VEGFR-1 under identical conditions was barely detectable. VEGFR-2 mediated signals cause striking changes in the morphology, actin reorganization, and membrane ruffling of porcine aortic endothelial cells recombinantly overexpressing this receptor. In these cells, VEGFR-2 also mediated ligand-induced chemotaxis and mitogenicity; whereas VEGFR-1-transfected cells lacked mitogenic responses to VEGF-A. Mutations in VEGF-A that disrupt binding to VEGFR-2 fail to induce proliferation of endothelial cells, whereas VEGF-A mutants that are deficient in binding VEGFR-1 are still capable of promoting endothelial proliferation. Similarly, VEGF stimulation of cells expressing only VEGFR-2 leads to a mitogenic response whereas comparable stimulation of cells expressing only VEGFR-1 can result in cell migration (e.g. in monocytes), but does not induce cell proliferation. In addition, phosphoproteins co-precipitating with VEGFR-1 and VEGFR-2 are distinct, suggesting that different signaling molecules interact with receptor-specific intracellular sequences.

The emerging hypothesis is that the primary function of VEGFR-1 in angiogenesis may be to negatively regulate the activity of VEGF-A by binding it and thus preventing its interaction with VEGFR-2, whereas VEGFR-2 is thought to be the main transducer of VEGF-A signals in endothelial cells. In support of this hypothesis, mice deficient in VEGFR-1 die as embryos while mice expressing a VEGFR-1 receptor capable of binding VEGF-A but lacking the tyrosine kinase domain survive and do not exhibit abnormal embryonic development or angiogenesis. In addition, analyses of VEGF-A mutants that bind only VEGFR-2 show that they retain the ability to induce mitogenic responses in endothelial cells. However, VEGF-mediated migration of monocytes is dependent on VEGFR-1, indicating that signaling through this receptor is important for at least one biological function. In addition, the ability of VEGF-A to prevent the maturation of dendritic cells is also associated with VEGFR-1 signaling, suggesting that VEGFR-1 may function in cell types other than endothelial cells. [Ferrara, J Mol Med 77:527-543 (1999); Zachary, Intl J Biochem Cell Bio 30:1169-1174 (1998)].

With respect to the VEGF-C polypeptides, neuropilins or other polypeptides used to practice the invention, it will be understood that native sequences will usually be most preferred. By “native sequences” is meant sequences encoded by naturally occurring polynucleotides, including but not limited to prepro-peptides, pro-peptides, and partially and fully proteolytically processed polypeptides. As described above, many of the polypeptides have splice variants that exist, e.g., due to alternative RNA processing, and such splice variants comprise native sequences. For purposes described herein, fragments of the forgoing that retain the binding properties of interest also shall be considered native sequences. Moreover, modifications can be made to most protein sequences without destroying the activity of interest of the protein, especially conservative amino acid substitutions, and proteins so modified are also suitable for practice of the invention. By “conservative amino acid substitution” is meant substitution of an amino acid with an amino acid having a side chain of a similar chemical character. Similar amino acids for making conservative substitutions include those having an acidic side chain (glutamic acid, aspartic acid); a basic side chain (arginine, lysine, histidine): a polar amide side chain (glutamine, asparagine); a hydrophobic, aliphatic side chain (leucine, isoleucine, valine, alanine, glycine); an aromatic side chain (phenylalanine, tryptophan, tyrosine); a small side chain (glycine, alanine, serine, threonine, methionine); or an aliphatic hydroxyl side chain (serine. threonine).

Moreover, deletion and addition of amino acids is often possible without destroying a desired activity. With respect to the present invention, where binding activity is of particular interest and the ability of molecules to activate or inhibit receptor tyrosine kinases upon binding is of special interest, binding assays and tyrosine phophorylation assays are available to determine whether a particular ligand or ligand variant (a) binds and (b) stimulates or inhibits RTK activity.

Candidate VEGF-C analog polypeptides can be rapidly screened first for their ability to bind and (with respect to certain receptors) stimulate autophosphorylation of VEGF-C receptors (VEGFR-2, VEGFR-3) or cellular activation through their receptors (VEGFR-2, VEGFR-3, NRP-1 and NRP-2). Polypeptides that stimulate these receptors are rapidly re-screened in vitro for their mitogenic and/or chemotactic activity against cultured capillary or arterial endothelial cells (e.g., as described in WO 98/33917). Polypeptides with mitogenic and/or chemotactic activity are then screened in vivo as described herein for efficacy in methods of the invention. In this way, variants (analogs) of naturally occurring VEGF-C proteins are rapidly screened to determine whether or not the variants have the requisite biological activity to constitute “VEGF-C polypeptides” for use in the present invention.

Two manners for defining genera of polypeptide variants include percent amino acid identity to a native polypeptide (e.g., 80, 85, 90, 91, 92, 93, 94, 95, 96, 97, 98, or 99% identity preferred), or the ability of encoding-polynucleotides to hybridize to each other under specified conditions. One exemplary set of conditions is as follows: hybridization at 42° C. in 50% formamide, 5× SSC, 20 mM Na.PO4, pH 6.8; and washing in 1× SSC at 55° C. for 30 minutes. Formula for calculating equivalent hybridization conditions and/or selecting other conditions to achieve a desired level of stringency are well known. It is understood in the art that conditions of equivalent stringency can be achieved through variation of temperature and buffer, or salt concentration as described Ausubel. et al. (Eds.). Protocols in Molecular Biology, John Wiley & Sons (1994). pp. 6.0.3 to 6.4.10. Modifications in hybridization conditions can be empirically determined or precisely calculated based on the length and the percentage of guanosine/cytosine (GC) base pairing of the probe. The hybridization conditions can be calculated as described in Sambrook, et al., (Eds.), Molecular Cloning: A Laboratory Manual, Cold Spring Harbor Laboratory Press: Cold Spring Harbor, N.Y. (1989), pp. 9.47 to 9.51.

B. Neural Stem Cells

The preset invention relates to the activation and proliferation of neural stem cells by vascular endothelial growth factor C and methods for using VEGF-C to stimulate neuronal growth and regeneration in the treatment of neuropathologies

Stem cells, also referred to as progenitor cells, comprise both embryonic and adult stem cells. Adult stems cells include, but are not limited to, neural stem cells, hematopoietic stem cells, endothelial stem cells, and epithelial stem cells. See Tepper, et al., Plastic and Reconstructive Surgery, 111:846-854 (2003). Endothelial progenitor cells circulated in the blood and migrate to regions characterized by injured endothelia. Kaushal, et al., Nat. Med., 7:1035-1040 (2001). A small subpopulation of human CD34(+)CD133(+) stem cells from different hematopioetic sources co-express VEGFR-3 (Salven, et al., Blood, 101(1): 168-72 (2003). These cells also have the capacity to differentiate to lymphatic and/or vascular endothelial cells in vitro.

The term “stem cell recruitment” refers to the ability to cause mobilization of stem cells (e.g., from bone marrow into circulation). The term “proliferation” refers to mitotic reproduction. The term “differentiation” refers to the process by which the pluripotent stem cells develop into other cell types. Differentiation may involve a number of stages between pluripotency and fully differentiated cell types.

The present invention further provides methodology for stimulating growth of neural cell populations. These neural cell populations, including neurons and glial derived cells, are used therapeutically to treat a subject exhibiting neuropathology. For example, the present invention is used to treat neurodegenerative diseases such as Alzheimer's disease or Parkinson's disease, or neuropathology resulting from insults such as during stroke, ischemia or surgery, or traumatic injury such as spinal cord injuries.

Neural stem cells (NSCs) are immature, uncommitted cells that exist in the developing, and even adult, CNS and are postulated to give rise to the array of specialized cells in the CNS. They are operationally defined by their ability to self-renew and to differentiate into cells of most (if not all) neuronal and glial lineages, and to populate developing and/or degenerating CNS regions [Ciage et al., Ann Rev Neurosci 18: 159-92, 1995; Whittemore et al., Mol. Neurobiology 12:13-39 1996; McKay Science 276: 66-71, 1997; Gage F H, Christen Y. (eds.), Research & Perspectives in Neurosciences: Isolation, Characterization, & Utilization of CNS Stem Cells, Springer-Verlag, Heidelberg, Berlin, 1997; Snyder, The Neuroscientist 4, 408-25, 1998].

Neural stem cells found in adult mammals are isolated primarily from the hippocampus, olfactory bulb and adult ventricular zone, as well as the spinal cord (Temple, S. Nature 414:112-117. 2001). Studies have demonstrated that precursor cells isolated from the hippocampus (esp. the subgranular zone of the dentate gyrus) of adult rodents proliferate in vitro when stimulated with epidermal growth factor or basic fibroblast growth factor, and upon transplantation to brain in vivo, migrate and differentiate into mature neurons (Gage et al., Proc. Natl. Acad. Sci. 92: 11879-83. 1995).

Examples of migrating stem cells useful according to the present invention include, but are not limited to, the C17.2 neuronal stem cell line (Riess et al., Neurosurgery. 51:1043-52.2002), purified neural stem cells, HSN-1 cells (human cerebral cortex), fetal pig cells and neural crest cells, bone marrow derived neural stem cells, hNT cells (human neuronal cell line), and a human neuronal progenitor cell line (Clonetics, Walkersville, Md., catalog number CC-2599). HSN-1 cells useful in the invention are prepared as described in, e.g., Ronnett et al., [Science 248, 603-605, 1990]. hNT cells useful in the invention are prepared as described in, e.g., Konobu et al. [Cell Transplant 7, 549-558, 1998]. The preparation of neural crest cells is described by Stemple and Anderson (U.S. Pat. No. 5,654,183), which is incorporated herein by reference. Briefly, neural crest cells from mammalian embryos are isolated from the region containing the caudal-most 10 somites and are dissected from early embryos (equivalent to gestational day 10.5 day in the rat). These tissue sections are transferred in a balanced salt solution to chilled depression slides, typically at 4° C., and treated with collagenase in an appropriate buffer solution such as Howard's Ringer's solution. After the neural tubes are free of somites and notochords, they are plated onto fibronectin (FN)-coated culture dishes to allow the neural crest cells to migrate from the neural tube. Twenty-four hours later, following removal of the tubes with a sharpened tungsten needle, the crest cells are removed from the FN-coated plate by treatment with a Trypsin solution, typically at 0.05%. The suspension of detached cells is then collected by centrifugation and plated at an appropriate density, generally 225 cells/100 mm dish in an appropriate chemically defined medium, such as Dulbecco's modified Eagle's medium with 4 mM L-glutamine adjusted to contain 1.5 g/L sodium bicarbonate, 4.5 g/L glucose, 90%; fetal bovine serum, 10%. The growth medium should be adjusted to pH 7.35 prior to filtration. See U.S. Pat. No. 5,196,315.

The specific growth factors and concentrations of particular additives are altered as needed to provide optimal growth to a particular culture of neural stem cells. The medium can also be used free of serum and contains components which permit the growth and self-renewal of neural crest stem cells. The culture dishes are coated with an appropriate substratum, typically a combination of FN and poly-D-lysine (PDL).

Neural crest cells as described above are isolated based on their cell surface expression of low-affinity nerve growth factor receptor (LNGFR) and nestin and lack of neuronal or glial lineage markers including glial fibrillary acidic protein (GFAP). Antibodies to these molecules are used to purify populations of neural crest cells.

Both the isolated neural crest cells cultured according to this method and the cells resulting from their differentiating into are used in the instant invention.

A “neural stem cell” as used herein is a neural progenitor cell which is proto-neuronal/proto glial. The term neural stem cell is used interchangeably with neuronal progenitor cell, neuronal cell, neuronal precursor cell, and neurosphere. During development, embryonic stem cells which are very primitive totipotent cells are thought to pass through a neural stem cell stage as they are developing into neural cells. Neural stem cells can be induced to differentiate into any neural cells including glia, oligodendrocytes, neurons, or astrocytes. Cells are characterized as multipotent neural progenitor cells based on the ability to propagate over many passages, expression of nestin and Ki-67, proto-neuronal morphology, as well as the ability to differentiate into neurons and glia. Sources of NSCs may be any tissue that contains NSCs, including but not limited to: brain, spinal cord, fetal tissue, retina, and embryo (see U.S. Patent Publ. No. 2003/0040023). Mammalian neural crest stem cells and multipotent neural stem cells and their progeny can be isolated from tissues from human and non-human primates, equines, canines, felines, bovines, porcines, etc.

Many differentiation agents or neurotrophic factors are known to one of skill in the art which can differentiate adult stem cells, embryonic stem cells, retinal stem cells, or neural stem cells into specific types of nerve cells, retina cells or types of progenitors. These neurotrophic factors include endogenous soluble proteins regulating survival, growth, morphological plasticity, or synthesis of proteins for differentiated functions of neurons. Therefore, it is envisioned that the stem cells isolated herein may be differentiated if so desired by any means known to one of skill in the art. Some examples of differentiation agents, include, but are not limited to Interferon gamma, fetal calf serum, nerve growth factor, removal of epidermal growth factor (EGF), removal of basic fibroblast growth factor (bFGF) (or both), neurogenin, brain derived neurotrophic factor (BDNF), thyroid hormone, bone morphogenic proteins (BMPs), LIF, sonic hedgehog, and glial cell line-derived neurotrophic factor (GDNFs), vascular endothelial growth factor (VEGF), interleukins, interferons, stem cell factor (SCF), activins, inhibins, chemokines, retinoic acid and CNTF. The cells may be differentiated permanently or temporarily. For example, cells may be differentiated temporarily to express a specific marker, for example, in order to use that marker for identification. Then, the differentiation agent may be removed and the marker may no longer be expressed.

It is contemplated that anti-differentiation agents may also be used as necessary to inhibit differentiation of progenitor cells and maintain totipotency. These anti-differentiation agents including but are not limited to: TGF-β, TGFα, EGF, FGFs, and delta (notch ligand).

The neural stem cells described above are useful in the treatment of neuropathologies via administration and transfer of these cells to a mammalian subject suffering from a disease or condition which requires neural cell regeneration. VEGF-C product or VEGF-D product is administered to these individuals to generate regrowth of neural stem cells in vivo, and is administered in any one of the methods described below. In one alternative method, VEGF-C product or VEGF-D product is administered to cells in culture to stimulate proliferation of the stem cells themselves, or to induce differentiation of a desired population of neural cell, which is then transplanted into the individual in need of therapy.

It is further contemplated that viral vectors carrying a VEGF-C or VEGF-D transgene and designed to infect mammalian cells and cause the cells to secrete VEGF-C or VEGF-D polypeptide are administered directly to a subject in need of therapy for neuropathology or alternatively, are transferred to neural stem cells in in vitro culture and then transplanted into the subject. The viral vectors are designed to secrete VEGF-C or VEGF-D and stimulate neural stem cell proliferation and ameliorate symptoms of neuropathology.

C. Neuropathological Indications and VEGF-C/VEGF-D Treatment Therapies

The peripheral nervous system (PNS) comprises both sensory neurons and motor neurons that connect the central nervous system (CNS) to the internal organs, such as heart, lungs, and glands. The peripheral nervous system is divided into the sensory nervous system and the autonomic nervous system, which is further subdivided into the sympathetic and parasympathetic nervous systems. The sympathetic nervous system is regulated by the neurotransmitters acetylcholine and norepinerphrine, which help regulate such basic functions as heartbeat, blood pressure, pupil dilation, swallowing mechanisms, liver activity, and movement of blood to muscles, heart and brain. Neurodegeneration of neurons or other supporting nervous system cells in the sympathetic nervous system can cause tremendous systemic difficulties. The disclosure herein that VEGF-C stimulates sympathetic nervous cell precursors in vitro to proliferate and grow points to VEGF-C as an emerging therapeutic to overcome the effects of these detrimental neuropathologies.

Recent discoveries in the field of neurology indicate that neural stem cells may be isolated from the adult hippocampus of mammals. The hippocampus is critically involved in learning and memory and is extremely vulnerable to insults such as brain trauma and ischemia. (Nakatomi et al. Cell 110:429-41, 2002). This region is often affected in neurodegenerative disease.

Neurodegenerative diseases are characterized by a progressive degeneration (i.e., nerve cell dysfunction and death) of specific brain regions, resulting in weakened motor function, and may lead to dampened cognitive skills and dementia. Examples of neurodegenerative disease include but are not limited to Alzheimer's disease, Parkinson's disease, ALS and motor neuron disease.

Alzheimer's disease is diagnosed as a progressive forgetfulness leading to dementia. The AD brain demonstrates diffuse cerebral atrophy with enlarged ventricles, resulting from neuronal loss. In general, neurons in the hippocampal region are primarily involved in the pathology of AD.

Parkinson's Disease is characterized by tremors and reduced motor neuron function, rigidity, and akinesia. These neurologic signs are due to malfunction of the major efferent projection of the substantia nigra, i.e., the nigrostriatal tract. The cell bodies of neurons in the dopaminergic system are the primary cells involved in PD progression. Examples of primary parkinsonian syndromes include Parkinson's disease (PD), progressive supranuclear palsy (PSP), and striatonigral degeneration (SND), which is included with olivopontocerebellear degeneration (OPCD) and Shy. Drager syndrome (SDS) in a syndrome known as multiple system atrophy (MSA).

Amyotrophic lateral sclerosis (ALS), often referred to as “Lou Gehrig's disease,” is a progressive neurodegenerative disease that attacks motor neurons in the brain and spinal cord. The progressive degeneration of the motor neurons in ALS eventually leads to their death, reducing the ability of the brain to initiate and control muscle movement.

Huntington's disease (HD), although a genetically heritable disease, results in the degeneration of neurons in the striatal medium spiny GABAergic neurons (Hickey et al., Prog Neuropsychopharmacol Biol Psychiatry. 27:255-65, 2003). This degeneration causes uncontrolled movements, loss of intellectual faculties, and emotional disturbance.

Cerebral palsy (CP) is another condition that may be treated by the method of the invention. CP syndromes are a group of related motor disorders with originating usually from either developmental abnormalities or perinatal or postnatal central nervous system (CNS) disorder damage occurring before age 5. CP is characterized by impaired voluntary movement.

Patients affected by any of the above disorders are treated with VEGF-C product or VEGF-D product either systemically, or preferably at the site of neuropathology, to stimulate the proliferation of neural stem cells in vivo. Alternatively, patients are administered neural stem cells isolated from a biological sample, from a commercial source or an immortalized neural stem cell, which has been treated in vitro with VEGF-C or VEGF-D product, including viral vectors expressing VEGF-C or VEGF-D. The neural stem cells are then administered to a patient with a neurodegenerative disorder or neural trauma such that they will migrate to the site of neural degeneration and proliferate. The administration is done either systemically or locally as described below.

A patient suffering from any of the above disorders can be treated at the earliest signs of disease symptoms, such as impaired motor function or impaired cognitive function, in order to halt the progression of neurodegeneration. It is also contemplated that VEGF-C/D or VEGF-C/D cultured neuronal precursor cells are administered to individuals in late stages of disease to slow the progression of the nervous system damage.

It is also contemplated by the invention that administration of the VEGF-C product or VEGF-D product in combination with a neurotherapeutic agent commonly used to treat neuropathologies will create a synergism of the two treatments, thereby causing marked improvement in patients receiving the combination therapy as compared to individuals receiving only a single therapy.

Neurodegenerative disorders are treatable by several classes of neurotherapeutics. Therapeutics include, but are not limited to the following drugs: secretin, amantadine hydrochloride, risperidone, fluvoxamine, clonidine, amisulpride, bromocriptine clomipramine and desipramine.

Neurotherapeutics commonly used to treat Alzheimer's disease include tacrine (Cognex), donepezil (Aricept), rivastigmine (Exelon), or galantamine (Reminyl) which may help prevent some symptoms from becoming worse for a limited time. Also, some medicines may help control behavioral symptoms of AD such as sleeplessness, agitation, wandering, anxiety, and depression. Additional therapies for AD are anti-inflammatory drugs such as non-steroidal anti-inflammatory drugs (NSAIDs), e.g. COX-2 inhibitors (Celebrex) and naproxen sodium. Other anti-inflammatory agents also used are salicylates, steroids, receptor site blockers, or inhibitors of complement activation

Pramipexole (mirapex) and levodopa are effective medications to treat motor symptoms of early Parkinson disease (PD). In vitro studies and animal studies suggest that pramipexole may protect and that levodopa may either protect or damage dopamine neurons. Neuroimaging offers the potential of an objective biomarker of dopamine neuron degeneration in PD patients. Coenzyme Q10, a neurotransmitter that is expressed at low levels in Parkinson's patients, is also used for treatment of PD. Levodopa can be combined with another drug such as carbidopa to aid in relieving the side effects of L-dopa. Other medications used to treat Parkinson's disease, either as solo agents or in combination, are Sinemet, Selegiline, (marketed as Eldepryl) may offer some relief from early Parkinson symptoms. Amantadine (Symmetrel) is an anti-viral drug that also provides an anti-Parkinson effect, and is frequently used to widen the “therapeutic window” for Levodopa when used in combination with Sinemet.

Benadryl, Artane, and Cogentine are brand names for anti-cholinergic agents that may be prescribed to treat tremors. Anticholinergics block the action of acetylcholine in the neuromuscular junction, thereby rebalancing it in relation to dopamine and reducing rigidity and tremor. While effective, these drugs can have side effects such as dry mouth, blurred vision, urinary retention and constipation which limits their use in older adults.

Ropinirole (Requip), Pramipexole (Mirapex), Bromocriptine (Parlodel) and Pergolide (Permax) are dopamine agonists. These drugs enter the brain directly at the dopamine receptor sites, and are often prescribed in conjunction with Sinemet to prolong the duration of action of each dose of levodopa. They may also reduce levodopa-induced involuntary movements called “dyskinesias”. The physician slowly titrates a dopamine agonist to a therapeutic level, then gradually decreases the levodopa dose to minimize dyskinesias. Apomorphine is a dopamine agonist often given as a continuous subcutaneous infusion or as a subcutaneous injection.

Tolcaponc (Tasmar) and Entacapone, are COMT (catechol-0-methyl-transterase) inhibitors. When COMT activity is blocked, dopamine remains in the brain for a longer period of time. Their mechanism of action is totally different than that of dopamine agonists.

Rilutek®, Myotrophin®, Coenzyme Q, Topiramate, Xaliproden and Oxandrolone are exemplary agents used in the treatment of ALS.

It is contemplated that treatment with VEGF-C either before, after or simultaneously with any of the above neurotherapeutics will enhance the effect of the neurotherapeutic agent, thereby reducing the amount of agent required by an individual and reducing unwanted side effects produced by multiple or large doses of neurotherapeutic.

In addition to neurodegenerative disease, it is contemplated that VEGF-C or VEGF-D is useful in the treatment of disease of the autonomic nervous system. Exemplary disease include: Shy Drager syndrome, which is characterized by multiple system atrophy and severe hypotension (Lamarre-Cliché et al., Can J Clin Pharmacol. 6:213-5. 1999); Adie's syndrome, which is characterized by tonic pupil and areflexia (Mak et al., J Clin Neurosci. 7:452.2000); Horner's syndrome, which affects the innervation of the eye (Patel et al., Optometry 74:245-56. 2003); familial dysautonomia, which affects cardiovascular regulation (Bernardi, et al., Am. J. Respir. Crit. Care Med. 167:141-9. 2003); and regional pain syndrome, which is characterized by pain and altered sensation (Tumer-Stokes, L. Disabil. Rehabil. 24:939-47. 2002).

As stated above, it is further contemplated that VEGF-C and VEGF-D products are useful in the treatment of physical damage to the nervous system. Trauma may be caused by physical injury of the brain and spinal cord or crush or cut injuries, such as abrasion, incision, contusion, puncture, compression, or other injury resulting from traumatic contact of a foreign object to the arm, hand or other parts of the body, and also includes temporary or permanent cessation of blood flow to parts of the nervous system.

D. Gene Therapy

Much of the application, including some of the examples, are written in the context of protein-protein interactions and protein administration. Genetic manipulations to achieve modulation of protein expression or activity is also specifically contemplated. For example, where administration of proteins is contemplated, administration of a gene therapy vector to cause the protein of interest to be produced in vivo also is contemplated. Where inhibition of proteins is contemplated (e.g., through use of antibodies or small molecule inhibitors), inhibition of protein expression in vivo by genetic techniques, such as knock-out techniques or anti-sense therapy, is contemplated.

Any suitable vector may be used to introduce a transgene of interest into an animal. Exemplary vectors that have been described in the literature include replication-deficient retroviral vectors, including but not limited to lentivirus vectors [Kim et al., J. Virol., 72(1): 811-816 (1998); Kingsman & Johnson, Scrip Magazine, October, 1998, pp. 43-46.]; adenoviral (see, for example, U.S. Pat. No. 5,824,544; U.S. Pat. No. 5,707,618; U.S. Pat. No. 5,792,453; U.S. Pat. No. 5,693,509; U.S. Pat. No. 5,670,488; U.S. Pat. No. 5,585,362; Quantin et al., Proc. Natl. Acad. Sci. USA, 89: 2581-2584 (1992); Stratford-Perricadet et al., J. Clin. Invest., 90: 626-630 (1992); and Rosenfeld et al., Cell, 68: 143-155 (1992)), retroviral (see, for example, U.S. Pat. No. 5,888,502; U.S. Pat. No. 5,830,725; U.S. Pat. No. 5,770,414; U.S. Pat. No. 5,686,278; U.S. Pat. No. 4,861,719), adeno-associated viral (see, for example, U.S. Pat. No. 5,474,935; U.S. Pat. No. 5,139,941; U.S. Pat. No. 5,622,856; U.S. Pat. No. 5,658,776; U.S. Pat. No. 5,773,289; U.S. Pat. No. 5,789,390; U.S. Pat. No. 5,834,441; U.S. Pat. No. 5,863,541; U.S. Pat. No. 5,851,521; U.S. Pat. No. 5,252,479; Gnatenko et al., J. Investig. Med., 45: 87-98 (1997), an adenoviral-adenoassociated viral hybrid (see, for example, U.S. Pat. No. 5,856,152) or a vaccinia viral or a herpesviral (see, for example, U.S. Pat. No. 5,879,934; U.S. Pat. No. 5,849,571; U.S. Pat. No. 5,830,727; U.S. Pat. No. 5,661,033; U.S. Pat. No. 5,328,688); Lipofectin-mediated gene transfer (BRL); liposomal vectors [See, e.g., U.S. Pat. No. 5,631,237 (Liposomes comprising Sendai virus proteins)]; and combinations thereof. All of the foregoing documents are incorporated herein by reference in the entirety. Replication-deficient adenoviral vectors, adeno-associated viral vectors and lentiviruses constitute preferred embodiments.

In embodiments employing a viral vector, preferred polynucleotides include a suitable promoter and polyadenylation sequence to promote expression in the target tissue of interest. For many applications of the present invention, suitable promoters/enhancers for mammalian cell expression include, e.g., cytomegalovirus promoter/enhancer [Lehner et al., J. Clin. Microbiol. 29:2494-2502 (1991); Boshart et al., Cell, 41:521-530 (1985)]; Rous sarcoma virus promoter [Davis et al. Hum. Gene Ther., 4:151 (1993)]; simian virus 40 promoter, long terminal repeat (LTR) of retroviruses, keratin 14 promoter, and α myosin heavy chain promoter.

In other embodiments, non-viral delivery is contemplated. These include calcium phosphate precipitation (Graham and Van Der Eb, Virology, 52:456-467 (1973); Chen and Okayama, Mol. Cell Biol., 7:2745-2752, (1987); Rippe, et al., Mol. Cell Biol., 10:689-695 (1990)), DEAE-dextran (Gopal, Mol. Cell Biol., 5:1188-1190 (1985)), electroporation (Tur-Kaspa, et al., Mol. Cell Biol. 6:716-718. (1986); Potter, et al, Proc. Nat. Acad. Sci. USA, 81:7161-7165, (1984)), direct microinjection (Harland and Weintraub, J. Cell Biol., 101:1094-1099 (1985)), DNA-loaded liposomes (Nicolau and Sene, Biochim. Biophys. Acta, 721:185-190 (1982); Fraley, et al., Proc. Natl. Acad. Sci. USA, 76:3348-3352 (1979); Felgner, Sci. Am., 276(6):102-6 (1997); Felgner, Hum. Gene Ther., 7(15):1791-3, (1996)), cell sonication (Fechheimer, et al., Proc. Natl. Acad. Sci. USA, 84:8463-8467 (1987)), gene bombardment using high velocity microprojectiles (Yang, et al., Proc. Natl. Acad. Sci. USA, 87:9568-9572 (1990)), and receptor-mediated transfection (Wu and Wu, J. Biol. Chem., 262:4429-4432 (1987); Wu and Wu, Biochemistry, 27:887-892 (1988); Wu and Wu, Adv. Drug Delivery Rev., 12:159-167 (1993)).

In a particular embodiment of the invention, the expression construct (or indeed the peptides discussed above) may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, “In Liver Diseases, Targeted Diagnosis And Therapy Using Specific Receptors And Ligands,” Wu, G., Wu, C., ed., New York: Marcel Dekker, pp. 87-104 (1991)). The addition of DNA to cationic liposomes causes a topological transition from liposomes to optically birefringent liquid-crystalline condensed globules (Radler, et al., Science, 275(5301):810-4, (1997)). These DNA-lipid complexes are potential non-viral vectors for use in gene therapy and delivery.

Also contemplated in the present invention are various commercial approaches involving “lipofection” technology. In certain embodiments of the invention, the liposome may be complexed with a hemagglutinating virus (HVJ). This has been shows to facilitate fusion with the cell membrane and promote cell entry of liposome-encapsulated DNA (Kaneda, et al., Science, 243:3±5-378 (1989)). In other embodiments, the liposome may be complexed or employed in conjunction with nuclear nonhistone chromosomal proteins (HMG-1) (Kato, et al., J. Biol. Chem., 266:3361-3364 (1991)). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention.

Other vector delivery systems that can be employed to deliver a nucleic acid encoding a therapeutic gene into cells include receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu (1993), supra).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu (1987), supra) and transferrin (Wagner, et al., Proc. Nat'l. Acad Sci. USA, 87(9):3410-3414 (1990)). Recently, a synthetic neoglycoprotein, which recognizes the same receptor as ASOR, has been used as a gene delivery vehicle (Ferkol, et al., FASEB J., 7:1081-1091 (1993); Perales, et al., Proc. Natl. Acad. Sci. USA 91:4086-4090 (1994)) and epidermal growth factor (EGF) has also been used to deliver genes to squamous carcinoma cells (Myers, EPO 0273085).

In other embodiments, the delivery vehicle may comprise a ligand and a liposome. For example, Nicolau, et al., Methods Enzymol., 149:157-176 (1987) employed lactosyl-ceramide, a galactose-terminal asialganglioside, incorporated into liposomes and observed an increase in the uptake of the insulin gene by hepatocytes. Thus, it is feasible that a nucleic acid encoding a therapeutic gene also may be specifically delivered into a particular cell type by any number of receptor-ligand systems with or without liposomes.

In another embodiment of the invention, the expression construct may simply consist of naked recombinant DNA or plasmids. Transfer of the construct may be performed by any of the methods mentioned above that physically or chemically permeabilize the cell membrane. This is applicable particularly for transfer in vitro, however, it may be applied for in vivo use as well. Dubensky, et al, Proc. Nat. Acad. Sci. USA, 81:7529-7533 (1984) successfully injected polyomavirus DNA in the form of CaPO₄ precipitates into liver and spleen of adult and newborn mice demonstrating active viral replication and acute infection. Benvenisty and Neshif, Proc. Nat. Acad. Sci. USA, 83:9551-9555 (1986) also demonstrated that direct intraperitoneal injection of CaPO₄ precipitated plasmids results in expression of the transfected genes.

Another embodiment of the invention for transferring a naked DNA expression construct into cells may involve particle bombardment. This method depends on the ability to accelerate DNA coated microprojectiles to a high velocity allowing them to pierce cell membranes and enter cells without killing them (Klein, et al., Nature, 327:70-73 (1987)). Several devices for accelerating small particles have been developed. One such device relies on a high voltage discharge to generate an electrical current, which in turn provides the motive force (Yang, et al., Proc. Natl. Acad. Sci USA, 87:9568-9572 (1990)). The microprojectiles used have consisted of biologically inert substances such as tungsten or gold beads.

Those of skill in the art are aware of how to apply gene delivery to in vivo and ex vivo situations. For viral vectors, one generally will prepare a viral vector stock. Depending on the type of virus and the titer attainable, one will deliver 1×10⁴, 1×10⁵, 1×10⁶, 1×10⁷, 1×10⁸, 1×10⁹, 1×10¹⁰, 1×10¹¹ or 1×10¹² infectious particles to the patient. Similar figures may be extrapolated for liposomal or other non-viral formulations by comparing relative uptake efficiencies. Formulation as a pharmaceutically acceptable composition is discussed below.

Various routes are contemplated for various cell types. For practically any cell, tissue or organ type, systemic delivery is contemplated. In other embodiments, a variety of direct, local and regional approaches may be taken. For example, the cell, tissue or organ may be directly injected with the expression vector or protein.

In a different embodiment, ex vivo gene therapy is contemplated. In an ex vivo embodiment, cells from the patient are removed and maintained outside the body for at least some period of time. During this period, a therapy is delivered, after which the cells are reintroduced into the patient.

Anti-sense polynucleotides are polynucleotides which recognize and hybridize to polynucleotides encoding a protein of interest and can therefore inhibit transcription or translation of the protein. Full length and fragment anti-sense polynucleotides may be employed. Methods for designing and optimizing antisense nucleotides are described in Lima et al., (J Biol Chem; 272:626-38. 1997) and Kurreck et al., (Nucleic Acids Res.; 30:1911-8. 2002). Additionally, commercial software is available to optimize antisense sequence selection and also to compare selected sequences to known genomic sequences to help ensure uniqueness/specificity for a chosen gene. Such uniqueness can be further confirmed by hybridization analyses. Antisense nucleic acids are introduced into cells (e.g., by a viral vector or colloidal dispersion system such as a liposome). It is contemplated that the VEGF-C antisense nucleic acid molecules comprise a sequence complementary to any integer number of nucleotides from the target sequence from about 10 to 500, preferably from about 10 to 50. VEGFR-C antisense molecule may comprises a complementary sequence at least about 10, 25, 50, 100, 250 or 500 nucleotides in length or complementary to an entire VEGF-C coding strand. The antisense nucleic acid binds to the target nucleotide sequence in the cell and prevents transcription or translation of the target sequence. Phosphorothioate and methylphosphonate antisense oligonucleotides are specifically contemplated for therapeutic use by the invention. The antisense oligonucleotides may be further modified by poly-L-lysine, transferrin polylysine, or cholesterol moieties at their 5′ end.

In one embodiment, RNA of the invention can be used for induction of RNA interference (RNAi), using double stranded (dsRNA) (Fire et al., Nature 391: 806-811. 1998) or short-interfering RNA (siRNA) sequences (Yu et al., Proc Natl Acad Sci USA. 99:6047-52.2002). “RNAi” is the process by which dsRNA induces homology-dependent degradation of complimentary mRNA. In one embodiment, a nucleic acid molecule of the invention is hybridized by complementary base pairing with a “sense” ribonucleic acid of the invention to form the double stranded RNA. The dsRNA antisense and sense nucleic acid molecules are provided that correspond to at least about 20, 25, 50, 100, 250 or 500 nucleotides or an entire VEGF-C coding strand, or to only a portion thereof. In an alternative embodiment, the siRNAs are 30 nucleotides or less in length, and more preferably 21- to 23-nucleotides, with characteristic 2- to 3-nucleotide 3′-overhanging ends, which are generated by ribonuclease III cleavage from longer dsRNAs. See e.g. Tuschl T. (Nat Biotechnol. 20:446-48. 2002).

Intracellular transcription of small RNA molecules can be achieved by cloning the siRNA templates into RNA polymerase III (Pol III) transcription units, which normally encode the small nuclear RNA (snRNA) U6 or the human RNAse P RNA H1. Two approaches can be used to express siRNAs: in one embodiment, sense and antisense strands constituting the siRNA duplex are transcribed by individual promoters (Lee, et al. Nat. Biotechnol. 20, 500-505, 2002); in an alternative embodiment, siRNAs are expressed as stem-loop hairpin RNA structures that give rise to siRNAs after intracellular processing (Brummelkamp et al. Science 296:550-553. 2002) (herein incorporated by reference).

The dsRNA/siRNA is most commonly administered by annealing sense and antisense RNA strands in vitro before delivery to the organism. In an alternate embodiment, RNAi may be carried out by administering sense and antisense nucleic acids of the invention in the same solution without annealing prior to administration, and may even be performed by administering the nucleic acids in separate vehicles within a very close timeframe. Nucleic acid molecules encoding fragments, homologs, derivatives and analogs of a VEGF-C or antisense nucleic acids complementary to a VEGF-C nucleic acid sequence are additionally provided.

Genetic control can also be achieved through the design of novel transcription factors for modulating expression of the gene of interest in native cells and animals. For example, the Cys2-His2 zinc finger proteins, which bind DNA via their zinc finger domains, have been shown to be amenable to structural changes that lead to the recognition of different target sequences. These artificial zinc finger proteins recognize specific target sites with high affinity and low dissociation constants, and are able to act as gene switches to modulate gene expression. Knowledge of the particular target sequence of the present invention facilitates the engineering of zinc finger proteins specific for the target sequence using known methods such as a combination of structure-based modeling and screening of phage display libraries [Segal et al., Proc Natl Acad Sci USA 96:2758-2763. (1999): Liu et al., Proc Natl Acad Sci USA 94:5525-30. (1997); Greisman and Pabo Science 275:657-61 (1997); Choo et al., J Mol Biol 273:525-32 (1997)]. Each zinc finger domain usually recognizes three or more base pairs. Since a recognition sequence of 18 base pairs is generally sufficient in length to render it unique in any known genome, a zinc finger protein consisting of 6 tandem repeats of zinc fingers would be expected to ensure specificity for a particular sequence [Segal et al., supra]. The artificial zinc finger repeats, designed based on target sequences, are fused to activation or repression domains to promote or suppress gene expression [Liu et al., supra]. Alternatively, the zinc finger domains can be fused to the TATA box-binding factor (TBP) with varying lengths of linker region between the zinc finger peptide and the TBP to create either transcriptional activators or repressors [Kim et al., Proc Natl Acad Sci USA 94:3616-3620.(1997). Such proteins, and polynucleotides that encode them, have utility for modulating expression in vivo in both native cells, animals and humans. The novel transcription factor can be delivered to the target cells by transfecting constructs that express the transcription factor (gene therapy), or by introducing the protein. Engineered zinc finger proteins can also be designed to bind RNA sequences for use in therapeutics as alternatives to antisense or catalytic RNA methods [McColl et al., Proc Natl Acad Sci USA 96:9521-6 (1999); Wu et al., Proc Natl Acad Sci USA 92:344-348 (1995)].

E. Antibodies

Antibodies are useful for modulating Neuropilin-VEGF-C interactions and VEGF-C mitogenic activity due to the ability to easily generate antibodies with relative specificity, and due to the continued improvements in technologies for adopting antibodies to human therapy. Thus, the invention contemplates use of antibodies (e.g., monoclonal and polyclonal antibodies, single chain antibodies, chimeric antibodies, bifunctional/bispecific antibodies, humanized antibodies, human antibodies, and complementary determining region (CDR)-grafted antibodies, including compounds which include CDR sequences which specifically recognize a polypeptide of the invention) specific for polypeptides of interest to the invention, especially neuropilins. VEGF receptors, and VEGF-C and VEGF-D proteins. Preferred antibodies are human antibodies which are produced and identified according to methods described in WO93/11236, published Jun. 20, 1993, which is incorporated herein by reference in its entirety. Antibody fragments, including Fab, Fab′, F(ab′)2, and Fv, are also provided by the invention. The term “specific for,” when used to describe antibodies of the invention, indicates that the variable regions of the antibodies of the invention recognize and bind the polypeptide of interest exclusively (i.e., able to distinguish the polypeptides of interest from other known polypeptides of the same family, by virtue of measurable differences in binding affinity, despite the possible existence of localized sequence identity, homology, or similarity between family members). It will be understood that specific antibodies may also interact with other proteins (for example, S. aureus protein A or other antibodies in ELISA techniques) through interactions with sequences outside the variable region of the antibodies, and in particular, in the constant region of the molecule. Screening assays to determine binding specificity of an antibody of the invention are well known and routinely practiced in the art. For a comprehensive discussion of such assays, see Harlow et al. (Eds), Antibodies A Laboratory Manual; Cold Spring Harbor Laboratory; Cold Spring Harbor, N.Y. (1988), Chapter 6. Antibodies of the invention can be produced using any method well known and routinely practiced in the art.

Bispecific antibodies are monoclonal, preferably human or humanized, antibodies that have binding specificities for at least two different antigens. In the present case, one of the binding specificities is for NRP-2, the other one is for an NRP-2 binding partner, and preferably for a cell-surface protein or receptor or receptor subunit, such as VEGFR-3.

In one embodiment, a bispecific antibody which binds to both NRP-2 and VEGFR-3 is used to modulate the growth, migration or proliferation of cells that results from the interaction of VEGF-C with VEGFR-3. For example, the bispecific antibody is administered to an individual having tumors characterized by lymphatic metastasis or other types of tumors expressing both VEGF-C and VEGFR-3, and NRP-2. The bisepcific antibody which binds both NRP-2 and VEGFR-3 blocks the binding of VEGF-C to VEGFR-3, thereby interfereing with VEGF-C mediated lymphangiogenesis and slowing the progression of tumor metastatsis. In another embodiment, the same procedure is carried out with a bispecific antibody which binds to NRP-2 and VEGF-C, wherein administration of said antibody sequesters soluble VEGF-C and prevents its binding to VEGFR-3, effectively acting as an inhibitor of VEGF-C mediated signaling through VEGFR-3.

Bispecific antibodies are produced, isolated, and tested using standard procedures that have been described in the literature. See. e.g., Pluckthun & Pack, Immunotechnology, 3:83-105 (1997); Carter et al., J. Hematotherapy, 4: 463-470 (1995); Renner & Pfreundschuh, Immunological Reviews, 1995, No. 145, pp. 179-209; Pfreundschuh U.S. Pat. No. 5,643,759; Segal et al., J. Hematotherapy, 4: 377-382 (1995); Segal et al., Immunobiology, 185: 390-402 (1992); and Bolhuis et al., Cancer Immunol. Immunother., 34: 1-8 (1991), all of which are incorporated herein by reference in their entireties.

The term “bispecific antibody” refers to a single, divalent antibody which has two different antigen binding sites (variable regions). As described below, the bispecific binding agents are generally made of antibodies, antibody fragments, or analogs of antibodies containing at least one complementarity determining region derived from an antibody variable region. These may be conventional bispecific antibodies, which can be manufactured in a variety of ways (Holliger, P. and Winter G. Current Opinion Biotechnol. 4, 446-449 (1993)), e.g. prepared chemically, using hybrid hybridomas, via linking the coding sequence of such a bispecific antibody into a vector and producing the recombinant peptide or by phage display. The bispecific antibodies may also be any bispecific antibody fragments.

In one method, bispecific antibodies fragments are constructed by converting whole antibodies into (monospecific) F(ab′)₂ molecules by proteolysis, splitting these fragments into the Fab′ molecules and recombine Fab′ molecules with different specificity to bispecific F(ab′)₂ molecules (see, for example, U.S. Pat. No. 5,798,229).

A bispecific antibody can be generated by enzymatic conversion of two different monoclonal antibodies, each comprising two identical L (light chain)-H (heavy chain) half molecules and linked by one or more disulfide bonds, into two F(ab′)₂ molecules, splitting each F(ab′)2 molecule under reducing conditions into the Fab′ thiols, derivatizing one of these Fab′ molecules of each antibody with a thiol activating agent and combining an activated Fab′ molecule bearing NRP-2 specificity with a non-activated Fab′ molecule bearing an NRP-2 binding partner specificity or vice versa in order to obtain the desired bispecific antibody F(ab′)₂ fragment.

As enzymes suitable for the conversion of an antibody into its F(ab′)₂ molecules, pepsin and papain may be used. In some cases, trypsin or bromelin are suitable. The conversion of the disulfide bonds into the free SH-groups (Fab′ molecules) may be performed by reducing compounds, such as dithiothreitol (DTT), mercaptoethanol, and mercaptoethylamine. Thiol activating agents according to the invention which prevent the recombination of the thiol half-molecules, are 5,5′-dithiobis(2-nitrobenzoic acid) (DTNB), 2,2′-dipyridinedisulfide, 4,4′-dipyridinedisulfide or tetrathionate/sodium sulfite (see also Raso et al., Cancer Res., 42:457 (1982), and references incorporated therein).

The treatment with the thiol-activating agent is generally performed only with one of the two Fab′ fragments. Principally, it makes no difference which one of the two Fab′ molecules is converted into the activated Fab′ fragment (e.g., Fab′-TNB). Generally, however, the Fab′ fragment being more labile is modified with the thiol-activating agent. In the present case, the fragments bearing the anti-tumor specificity are slightly more labile, and, therefore, preferably used in the process. The conjugation of the activated Fab′ derivative with the free hinge-SH groups of the second Fab′ molecule to generate the bivalent F(ab′)₂ antibody occurs spontaneously at temperatures between 0° and 30° C. The yield of purified F(ab′)₂ antibody is 20-40% (starting from the whole antibodies).

Another method for producing bispecific antibodies is by the fusion of two hybridomas to form a hybrid hybridoma. As used herein, the term “hybrid hybridoma” is used to describe the productive fusion of two B cell hybridomas. Using now standard techniques, two antibody producing hybridomas are fused to give daughter cells, and those cells that have maintained the expression of both sets of clonotype immunoglobulin genes are then selected.

To identify the bispecific antibody standard methods such as ELISA are used wherein the wells of microtiter plates are coated with a reagent that specifically interacts with one of the parent hybridoma antibodies and that lacks cross-reactivity with both antibodies. In addition, FACS, immunofluorescence staining, idiotype specific antibodies, antigen binding competition assays, and other methods common in the art of antibody characterization may be used in conjunction with the present invention to identify preferred hybrid hybridomas.

Bispecific molecules of this invention can also be prepared by conjugating a gene encoding a binding specificity for NRP-2 to a gene encoding at least the binding region of an antibody chain which recognizes a binding partner of NRP-2 such as VEGF-C or VEGFR-3. This construct is transfected into a host cell (such as a myeloma) which constitutively expresses the corresponding heavy or light chain, thereby enabling the reconstitution of a bispecific, single-chain antibody, two-chain antibody (or single chain or two-chain fragment thereof such as Fab) having a binding specificity for NRP-2 and for a NRP-2 binding partner. Construction and cloning of such a gene construct can be performed by standard procedures.

Bispecific antibodies are also generated via phage display screening methods using the so-called hierarchical dual combinatorial approach as disclosed in WO 92/01047 in which an individual colony containing either an H or L chain clone is used to infect a complete library of clones encoding the other chain (L or H) and the resulting two-chain specific binding member is selected in accordance with phage display techniques such as those described therein. This technique is also disclosed in Marks et al., (Bio/Technology, 1992, 10:779-783).

The bispecific antibody fragments of the invention can be administered to human patients for therapy. Thus, in one embodiment the bispecific antibody is provided with a pharmaceutical formulation comprising as active ingredient at least one bispecific antibody fragment as defined above, associated with one or more pharmaceutically acceptable carrier, excipient or diluent. In another embodiment, the compound further comprises an anti-neoplastic or cytotoxic agent conjugated to the bispecific antibody.

Recombinant antibody fragments, e.g. scFvs, can also be engineered to assemble into stable multimeric oligomers of high binding avidity and specificity to different target antigens. Such diabodies (dimers), triabodies (trimers) or tetrabodies (tetramers) are well known within the art and have been described in the literature, see e.g. Kortt et al., Biomol Eng. 2001 Oct. 15;18(3):95-108 and Todorovska et al., J Immunol Methods. 2001 Feb. 1;248(1-2):47-66.

In addition to the production of monoclonal antibodies, techniques developed for the production of “chimeric antibodies”, the splicing of mouse antibody genes to human antibody genes to obtain a molecule with appropriate antigen specificity and biological activity, can be used (Morrison et al. Proc Natl Acad Sci 81: 6851-6855, 1984; Neuberger et al., Nature 312: 604-608. 1984; Takeda et al. Nature 314: 452-454; 1985).

Non-human antibodies may be humanized by any methods known in the art. A preferred “humanized antibody” has a human constant region, while the variable region, or at least a CDR, of the antibody is derived from a non-human species. Methods for humanizing non-human antibodies are well known in the art. (see U.S. Patent Nos. 5,585,089, and 5,693,762). Generally, a humanized antibody has one or more amino acid residues introduced into its framework region from a source which is non-human. Humanization can be performed, for example, using methods described in Jones et al. [Nature 321: 522-525, (1986)], Riechmann et al., [Nature, 332: 323-327, (1988)] and Verhoeyen et al. [Science 239:1534-1536, (1988)], by substituting at least a portion of a rodent complementarity-determining region (CDRs) for the corresponding regions of a human antibody. Numerous techniques for preparing engineered antibodies are described, e.g., in Owens and Young, J. Immunol. Meth., 168:149-165 (1994). Further changes can then be introduced into the antibody framework to modulate affinity or immunogenicity.

F. Formulation of Pharmaceutical Compositions

The VEGF-C products are preferably administered in a composition with one or more pharmaceutically acceptable carriers. Pharmaceutical carriers used in the invention include pharmaceutically acceptable salts, particularly where a basic or acidic group is present in a compound. For example, when an acidic substituent, such as —COOH, is present, the ammonium, sodium, potassium, calcium and the like salts, are contemplated as preferred embodiments for administration to a biological host. When a basic group (such as amino or a basic heteroaryl radical, such as pyridyl) is present, then an acidic salt, such as hydrochloride, hydrobromide, acetate, maleate, pamoate, phosphate, methanesulfonate, p-toluenesulfonate, and the like, is contemplated as a preferred form for administration to a biological host.

Similarly, where an acid group is present, then pharmaceutically acceptable esters of the compound (e.g., methyl, tert-butyl, pivaloyloxymethyl, succinyl, and the like) are contemplated as preferred forms of the compounds, such esters being known in the art for modifying solubility and/or hydrolysis characteristics for use as sustained release or prodrug formulations.

In addition, some compounds may form solvates with water or common organic solvents. Such solvates are contemplated as well.

Pharmaceutical VEGF-C product compositions can be used directly to practice materials and methods of the invention, but in preferred embodiments, the compounds are formulated with pharmaceutically acceptable diluents, adjuvants, excipients, or carriers. The phrase “pharmaceutically or pharmacologically acceptable” refer to molecular entities and compositions that do not produce adverse, allergic, or other untoward reactions when administered to an animal or a human, e.g., orally, topically, transdermally, parenterally, by inhalation spray, vaginally, rectally, or by intracranial injection. (The term parenteral as used herein includes subcutaneous injections, intravenous, intramuscular, intracisternal injection, or infusion techniques. Administration by intravenous, intradermal, intramuscular, intramammary, intraperitoneal, intrathecal, retrobulbar, intrapulmonary injection and or surgical implantation at a particular site is contemplated as well.) Generally, this will also entail preparing compositions that are essentially free of pyrogens, as well as other impurities that could be harmful to humans or animals. The term “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art.

The pharmaceutical compositions containing the VEGF-C products described above may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any known method, and such compositions may contain one or more agents selected from the group consisting of sweetening agents, flavoring agents, coloring agents and preserving agents in order to provide pharmaceutically elegant and palatable preparations. Tablets may contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid: binding agents, for example starch, gelatin or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for controlled release.

Formulations for oral use may also be presented as hard gelatin capsules wherein the active ingredient is mixed with an inert solid diluent, for example, calcium carbonate, calcium phosphate or kaolin, or as soft gelating capsules wherein the active ingredient is mixed with water or an oil medium, for example peanut oil, liquid paraffin, or olive oil.

Aqueous suspensions may contain the active compounds in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be presented by the addition of an anti-oxidant such as ascorbic acid.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents. The pharmaceutical compositions may be in the form of a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compositions may also be in the form of suppositories for rectal administration of the PTPase modulating compound. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols, for example.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

G. Administration and Dosing

Some methods of the invention include a step of polypeptide administration to a human or animal. Polypeptides may be administered in any suitable manner using an appropriate pharmaceutically-acceptable vehicle, e.g., a pharmaceutically-acceptable diluent, adjuvant, excipient or carrier. The composition to be administered according to methods of the invention preferably comprises (in addition to the polynucleotide or vector) a pharmaceutically-acceptable carrier solution such as water, saline, phosphate-buffered saline, glucose, or other carriers conventionally used to deliver therapeutics or imaging agents.

The “administering” that is performed according to the present invention may be performed using any medically-accepted means for introducing a therapeutic directly or indirectly into a mammalian subject, including but not limited to injections (e.g., intravenous, intramuscular, subcutaneous, intracranial or catheter); oral ingestion; intranasal or topical administration; and the like. For administration to a subject with neuronal disease, it is contemplated that the cells are injected into an area containing various peripheral nerves known to be effected in a particular mammal or into the spinal cord or brain for mammals which show involvement of the nervous system (Craig et al., J Neurosci. 1996 16:2649-58; Frisen et al., CMLS Cell. Mol. Life Sci. 54:935-45. 1998). In one embodiment, administering the composition is performed at the site of a lesion or affected tissue needing treatment by direct injection into the lesion site or via a sustained delivery or sustained release mechanism, which can deliver the formulation internally. For example, biodegradable microspheres or capsules or other biodegradable polymer configurations capable of sustained delivery of a composition (e.g., a soluble polypeptide, antibody, or small molecule) can be included in the formulations of the invention implanted near the lesion.

The therapeutic composition may be delivered to the patient at multiple sites. The multiple administrations may be rendered simultaneously or may be administered over a period of several hours. In certain cases it may be beneficial to provide a continuous flow of the therapeutic composition. Additional therapy may be administered on a period basis, for example, daily, weekly or monthly.

Polypeptides for administration may be formulated with uptake or absorption enhancers to increase their efficacy. Such enhancer include for example, salicylate, glycocholate/linoleate, glycholate, aprotinin, bacitracin, SDS caprate and the like. See, e.g., Fix (J. Pharm. Sci., 85:1282-1285, 1996) and Oliyai and Stella (Ann. Rev. Pharmacol. Toxicol., 32:521-544, 1993).

The amounts of peptides in a given dosage will vary according to the size of the individual to whom the therapy is being administered as well as the characteristics of the disorder being treated. In exemplary treatments, it may be necessary to administer about 50 mg/day, 75 mg/day, 100 mg/day, 150 mg/day, 200 mg/day, 250 mg/day, 500 mg/day or 1000 mg/day. These concentrations may be administered as a single dosage form or as multiple doses. Standard dose-response studies, first in animal models and then in clinical testing, reveal optimal dosages for particular disease states and patient populations.

It will also be apparent that dosing should be modified if traditional therapeutics are administered in combination with therapeutics of the invention. For example, treatment of neuropathology using traditional neurotherapeutic agents or nerve growth factors, in combination with methods of the invention, is contemplated.

H. Kits

As an additional aspect, the invention includes kits which comprise one or more compounds or compositions of the invention packaged in a manner which facilitates their use to practice methods of the invention. In a simplest embodiment, such a kit includes a compound or composition described herein as useful for practice of a method of the invention (e.g., polynucleotides or polypeptides for administration to a person or for use in screening assays), packaged in a container such as a sealed bottle or vessel, with a label affixed to the container or included in the package that describes use of the compound or composition to practice the method of the invention. Preferably, the compound or composition is packaged in a unit dosage form. The kit may further include a device suitable for administering the composition according to a preferred route of administration or for practicing a screening assay.

Additional aspects and details of the invention will be apparent from the following examples, which are intended to be illustrative rather than limiting.

### EXAMPLE 1

### VEGF-C Isoforms Bind to Neuropilin-2 and Neuropilin-1

The following experiments demonstrated that VEGF-C isoforms interact with the neuropilin family members, neuropilin-2 and neuropilin-1.

A. Materials

To investigate the binding of neuropilin-2 to VEGF-C the following constructs were either made or purchased from commercial sources:

a) Cloning of the NRP-2/IgG expression vector. The extracellular domain of hNRP-2 was cloned into the plgplus vector in frame with the human IgG1 Fc tail as follows. Full-length NRP-2 cDNA (SEQ ID NO. 3) was assembled from several IMAGE Consortium cDNA Clones (Incyte Genomics) (FIG. 1A). The Image clones used are marked as 2A (GenBank Acc. No AA621145; Clone ID 1046499). 3 (AA931763; 1564852), 4 (AA127691; 490311), and 5 (AW296186; 2728688); these clones were confirmed by sequencing. Image clones 4 and 5 differ due to alternative splicing, coding for a17 and a22 isoforms, respectively. The BamHI-NotI fragment from the image clone 3 was first cloned into the pcDNA3.1z+ vector (Invitrogen), and fragments KpnI-BglII from clone 2A and BgIII-BamHI from clone 3 were then added to obtain the 5′ region (bp 1-2188). NotI-BamHI fragments from clones 4 and 5 were separately transferred into the plgplus vector, and the KpnI-NotI fragment from the pcDNA3.1 z+ vector was then inserted to obtain the expression vector coding for the extracellular domain of the hNRP-2/IgG fusion protein (SEQ ID NO. 3, positions 1 to 2577). The NRP-2 inserts in the resulting vectors were sequenced. The Image clone 3 codes for one amino acid different from the GenBank Sequence (AAA 1804-1806 GAG | K602E). However, the amino acid sequence in the Image clone 3 is identical to the original sequence published by Chen et al. (Chen et al., Neuron, 19:547. 1997).

b) a VEGFR-3-Fc construct, in which an extracellular domain portion of VEGFR-3 comprising the first three immunoglobulin-like domains (SEQ ID NO. 32, amino acids 1 to 329) was fused to the Fc portion of human IgG1 [see Makinen et al., Nat. Med., 7:199-205 (2001)]. Full length VEGFR-3 cDNA and amino acid sequences are set forth in SEQ. ID NOS: 31 and 32.

c) a NRP-1-Fc construct, in which an extracellular domain portion of murine NRP-1 (base pairs 248-2914 of SEQ. ID NO: 5) was fused to the Fc portion of human IgG1 (Makinen et al, J. Biol. Chem 274:21217-222. 1999); and

d) the expression vectors, in pREP7 backbone, encoding either VEGF165 (Genbank Accession No. M32997) or full-length VEGF-C (SEQ. ID NO: 24), have been described recently (Olofsson et al., Proc. Natl. Acad. Sci. USA 93: 2576-81.1996; and Joukov et al., EMBO J. 15: 290-298.1996).

B. Co-Immunoprecipitation of VEGF-C with NRP-2

The NRP-2, NRP-1, and VEGFR-3 plgplus fusion constructs were transfected into 293T cells using the FUGENETM6 transfection reagent (Roche Molecular Biochemicals). The cells were grown in Dulbecco's modified Eagle's medium supplemented with 10% fetal calf serum (Gibco BRL), glutamine, and antibiotics. The media was replaced 48 h after transfection by DMEM containing 0.2% BSA and collected after 20 h.

For growth factor production. 293EBNA cells were transfected with expression vectors coding for VEGF₁₆₅, prepro-VEGF-C, or empty vector (Mock). 36 h after transfection, the cells were first incubated in methionine and cysteine free MEM (Gibco BRL) for 45 min, metabolically labeled in the same medium supplemented with 100 millicurie [mCi]/ml Pro-mix [³⁵S] (Amersham) for 6-7 h (1 mCi=37 kBq) containing radiolabelled methionine and cysteine.

For immunoprecipitation controls, 1 ml of the labeled medium was incubated with either MAB 293 monoclonal anti-VEGF-Ab (R&D Systems), or rabbit antiserum 882 against VEGF-C (Joukov et al., EMBO J. 16:3898-3911. 1997) for 2 h, with rotation, at +4° C. Protein A-Sepharose (Pharmacia) was then added, and incubated overnight. The immunoprecipitates were washed two times with ice-cold PBS-0.5% Tween 20, heated in Laemmli sample buffer, and electrophoresed in 15% SDS PAGE. The gel was dried and exposed to Kodak Biomax MR film.

For binding experiments, the labeled supernatants from the Mock- or VEGF-C transfected cells were first immunoprecipitated with VEGF antibodies (R & D Systems) for depletion of endogenous VEGF. 4 ml of hNRP-2 a17-IgG or 1 ml of VEGFR-3-IgG or NRP-1-IgG fusion protein containing media were incubated with 1 ml of growth factor containing media (Mock, VEGF or VEGF-C) in binding buffer (0.5% BSA, 0.02% Tween 20) for 2 h, Protein A-Sepharose was added, and incubated overnight. The samples were then washed once with ice-cold binding buffer and three times with PBS and subjected to 15% SDS PAGE. The radiolabeled VEGF-C polypeptide was detected via chemiluminescence (ECL).

Results show that both the 29 kD isoform and 21-23 kD VEGF-C isoform (as a heterodimer) bind to NRP-2 while only the 29 kD form binds to NRP-1. VEGFR-3 binding to VEGF-C was used as a positive control for VEGF-C binding in the assay. It has been shown previously that heparin strongly increases VEGF binding to NRP-2 (Gluzman-Poltorak et al., J. Biol. Chem. 275: 18040-045. 2000). Addition of heparin to the assay mixture illustrates that VEGF₁₆₅ binding to NRP-2 is heparin dependent while VEGF₁₆₅ binding to NRP-1 is independent of heparin binding, and the presence of heparin has no effect on VEGF-C binding to any of its receptors.

c. CELL-Based Assay Using Cells that Naturally Express Neuropilin Receptors.

The preceding experiment can be modified by substituting cells that naturally express a neuropilin receptor (especially NRP-2) for the transfected 293EBNA cells. Use of primary cultures of neural cells expressing neuropilin receptors is specifically contemplated, e.g., cultured cerebellar granule cells derived from embryos. Additionally, NRP-receptor-specific antibodies can be employed to identify other cells (e.g., cells involved in the vasculature), such as human microvascular endothelial cells (HMVEC), human cutaneous fat pad microvascular cells (HUCEC) that express NRP receptors.

### EXAMPLE 2

### Neuropilin-2 Interacts with VEGFR-3

Recent results indicate that NRP-1 is a co-receptor for VEGF₁₆₅ binding, forming a complex with VEGFR-2, which results in enhanced VEGF₁₆₅ signaling through VEGFR-2, over VEGF₁₆₅ binding to VEGFR-2 alone, thereby enhancing the biological responses to this ligand (Soker et al., Cell 92: 735-45.1998). A similar phenomenon may apply to VEGF-C signaling via possible VEGFR-3/NRP-2 receptor complexes.

A. Binding Assay

The NRP-2(a22) expression vector was cloned as described in Example 1 (FIG. 1B) with the addition of a detectable tag on the 3′ end. For 3′ end construction, the Not I-Bam HI fragment (clone 5) was then constructed by PCR, introducing the V5 tag (GKPIPNPLLGLDST) (SEQ ID NO:33) and a stop codon to the 3′ terminus. To obtain the expression vector coding for the full-length hNRP-2(a22) protein, this 3′ end was then transferred into the vector containing the 5′ fragment. The resulting clone was referred to as V5 NRP-2.

To determine the interaction of VEGFR-3 with NRP-2, 10 cm plates of human embryonic kidney cells (293T or 293EBNA) were transfected with the V5 NRP-2 construct or VEGFR-3 using 6 μl of FUGENE TM6 (Roche Molecular Biochemicals, Indianapolis, Ind.) and 2 μg DNA. The cells were grown in Dulbecco's modified Eagle's medium supplemented with 10% fetal calf serum (Gibco BRL), glutamine, and antibiotics. For Mock transfections, 2 μg of empty vector was used. For single receptor transfections, the VEGFR-3-myc/pcDNA3.1 (Karkkainen et al, Nat. Genet. 25:153-59.2000) or NRP-2(a22)/pcDNA3.1z+ and empty vector were used in a one to one ratio. The VEGFR-3/NRP-2 co-transfections were also made in a one to one ratio. After 24 h, the 293EBNA cells were starved overnight, and stimulated for 10 min using 300 ng/ml ΔNΔCVEGF-C (produced in P. pastoris; (Joukov et al. EMBOJ. 16: 3898-3911. 1997)). The cells were then washed twice with ice-cold PBS containing vanadate (100 μM) and PMSF (100 μM). and lysed in dimerization lysis buffer (20 mM HEPES pH 7.5, 150 mM NaCl,10% glycerol,1% Triton X-100.2 mM.MgCl2, 2 mM CaC12.10 μg/ml bovine serum albumin (BSA)) containing 2 mM vanadate. 1 mM PMSF. 0.07 U/ml aprotinin, and 4 μg/ml leupeptin. The lysates were cleared by centrifugation for 10 min at 19,000 g, and incubated with antibodies for VEGFR-3 (9d9F;(Jussila et al., Cancer Res. 58: 1599-1604. 1998)), or V5 (Invitrogen) for 5 h at +4° C. The immunocomplexes were then incubated with protein A-Sepharose (Pharmacia) overnight at +4° C., the immunoprecipitates were washed four times with dimerization lysis buffer without BSA, and the samples subjected to 7.5% SDS-PAGE in reducing conditions. The proteins were transferred to a Protran nitrocellulose filter (Schleicher & Schuell) using semi-dry transfer apparatus. After blocking with 5% non-fat milk powder in TBS-T buffer (10 mM Tris pH 7.5, 150 mM NaCl, 0.1% Tween 20), the filters were incubated with the V5 antibodies, followed by HRP-conjugated rabbit-anti-mouse immunoglobulins (Dako), and visualized using enhanced chemiluminescence (ECL).

Co-immunoprecipitation of VEGFR-3 and NRP-2 constructs transfected into 293T cells demonstrates that NRP-2 interacts with VEGFR-3 when co-expressed in the same cell. Immunoprecipitation after the addition of VEGF-C to the cell culture media shows that the NRP-21VEGFR-3 interaction is not dependent on the presence of the VEGF-C ligand, implying that these receptors may associate naturally in vivo without the presence of VEGF-C. This finding may have tremendous implications on the binding and activity of VEGF-C during angiogenesis. VEGF-C, an integral molecule in promoting growth and development of the lymphatic vasculature, is also highly involved in the metastasis of cancerous cells through the lymph system and apparently the neovascularization of at least some solid tumors (see International Patent Publication No. WO 00/21560). The novel interaction between neuropilins and VEGF-C provides for a means to specifically block this lymphatic growth into solid tumors by inhibiting lymphatic cell migration as a result of VEGF-C binding to VEGFR-3. Neuropilins-1 and -2 are the only VEGF receptors at the surface of some tumor cells, indicating the binding of VEGF to neuropilins is relevant to tumor growth (Soker et al, Cell 92: 735-45. 199S) and that VEGF-C binding to neuropilin-2 may be a means to specifically target tumor metastasis through the lymphatic system.

### EXAMPLE 3

### Inhibition of VEGF-C Binding to VEGFR-3 by Neuropilins

The binding affinity between VEGF-C and neuropilin receptor molecules provides therapeutic indications for modulators of VEGF-C-induced VEGFR-3 receptor signaling, in order to modulate, i.e. stimulate or inhibit, VEGF-receptor-mediated biological processes. The following examples are designed to provide proof of this therapeutic concept.

A. In Vitro Cell-Free Assay

To demonstrate the inhibitory effects of neuropilin-1-Fc and neuropilin-2-Fc against VEGF-C stimulation, a label, e.g. a biotin molecule, is fused with the VEGF-C protein and first incubated with neuropilin-1-Fc, neuropilin-2-Fc, VEGFR-2 Fc or VEGFR-3-Fc at various molar ratios, and then applied on microtiter plates pre-coated with 1 microgram/ml of VEGFR-3 or VEGFR-2. After blocking with 1% BSA/PBS-T, fresh, labeled VEGF-C protein or the VEGF-C/receptor-Fc mixture above is applied on the microtiter plates overnight at 4 degrees Centigrade. Thereafter, the plates are washed with PBS-T, and 11:1000 of avidin-HRP will be added. Bound VEGF-C protein is detected by addition of the ABTS substrate (KPL). The bound labeled VEGF-C is analyzed in the presence and absence of the soluble neuropilins or soluble VEGFRs and the percent inhibition of binding assessed, as well as the effects the neuropilins have on binding to either VEGFR-2 or VEGFR-3 coated microtiter plates. In a related variation, this assay is carried out substituting VEGF-D for VEGF-C.

B. In Vitro Cell-Based Assay

VEGF-C is used as described above to contact cells that naturally or recombinantly express NRP-2 and VEGFR-3 receptors on their surface. By way of example, 293EBNA or 293T cells recombinantly modified to transiently or stably express neuropilins and VEGFR-3 as outlined above are employed. Several native endothelial cell types express both receptors and can also be employed, including but not limited to, human microvascular endothelial cells (HMEC) and human cutaneous fat pad microvascular cells (HUCEC).

For assessment of autophosphorylation of VEGFR-3, 293T or 293EBNA human embryonic kidney cells grown in Dulbecco's modified Eagle's medium (DMEM) supplemented with 10% fetal calf serum (GIBCO BRL), glutamine and antibiotics, are transfected using the FUGENE TM6 transfection reagent (Roche Molecular Biochemicals) with plasmid DNAs encoding the receptor constructs (VEGFR-3 or VEGFR-3-myc tag and/or neuropilin-V5 tag,) or an empty pcDNA3.1z+ vector (Invitrogen). For stimulation assay, the 293EBNA cell monolayers are starved overnight (36 hours after transfection) in serum-free medium containing 0.2% BSA. The 293EBNA cells are then stimulated with 300 ng/ml recombinant DNDC VEGF-C (Joukov et al., EMBO J. 16:3898-3911. 1997) for 10 min at +37° C., in the presence or absence of neuropilin-Fc to determine inhibition of VEGF-C/VEGFR-3 binding. The cells are then washed twice with cold phosphate buffered saline (PBS) containing 2 mM vanadate and 2 mM phenylmethylsulfonyl fluoride (PMSF), and lysed into PLCLB buffer (150 mM NaCl, 5% glycerol, 1% Triton X-100, 1.5 M MgC12, and 50 mM Hepes, pH 7.5) containing 2 mM Vanadate, 2 mM PMSF, 0.07 U/ml Aprotinin, and 4 mg/ml leupeptin. The lysates are centrifuged for 10 min at 19 000 g, and incubated with the supernatants for 2 h on ice with 2 μg/ml of monoclonal anti-VEGFR-3 antibodies (9D9f9) (Jussila et al., Cancer Res. 58:1599-1604. 1998), or alternatively with antibodies against the specific tag epitopes (1.1 mg/ml of anti-V5 antibodies (Invitrogen) or 5 μg/ml anti-Myc antibodies (BabCO). The immunocomplexes are incubated with protein A sepharose (Pharmacia) for 45 min with rotation at +4° C. and the sepharose beads washed three times with cold PLCLB buffer (2 mM vanadate, 2 mM PMSF). The bound polypeptides are separated by 7.5% SDS-PAGE and transferred to a Protran nitrocellulose filter (Schleicher & Schuell) using semi-dry transfer apparatus. After blocking with 5% BSA in TBS-T buffer (10 mM Tris pH 7.5, 150 mM NaCl, 0.1% Tween 20), the filters are stained with the phosphotyrosine-specific primary antibodies (Upstate Biotechnology), followed by biotinylated goat-anti-mouse immunoglobulins (Dako) and Biotin-Streptavidin HRP complex (Amersham) Phosphotvrosine-specific bands are visualized by enhanced chemiluminescence (ECL). To analyze the samples for the presence of VEGFR-3, the filters are stripped for 30 min at +55° C. in 100 mM 2-mercaptoethanol, 2% SDS, 62.5 mM Tris-HCl pH 6.7 with occasional agitation, and stained with 9D9f9 antibodies and HRP conjugated rabbit-anti-mouse immunoglobulins (Dako) for antigen detection. Reduced VEGFR-3 autophosphorylation is indicative of successful neuropilin-Fc-mediated inhibition of VEGF-C/VEGFR3 binding.

VEGF-C protein naturally secreted into media conditioned by a PC-3 prostatic adenocarcinoma cell line (ATCC CRL 1435) in serum-free Ham's F-12 Nutrient mixture (GIBCO) (containing 7% fetal calf serum (FCS)) (U.S. Pat. No. 6,221,839) can be used to activate VEGFR3 expressing cells in vitro. For in vitro assay purposes, cells can be reseeded and grown in this medium, which is subsequently changed to serum-free medium. As shown in a previous experiment, pretreatment of the concentrated PC-3 conditioned medium with 50 microliters of VEGFR-3 extracellular domain coupled to CNBr-activated sepharose CL-4B (Pharmacia; about 1 mg of VEGFR-3EC domain/ml sepharose resin) completely abolished VEGFR-3 tyrosine phosphorylation (U.S. Pat. No. 6,221,839). In a related experiment, the PC-3 conditioned media can be pre-treated with a neuropilin composition or control Fc coupled to sepharose. The cells can be lysed, immunoprecipitated using anti-VEGFR-3 antiserum, and analyzed by Western blot using anti-phosphotyrosine antibodies as previously described. The percent inhibition of VEGF-C binding and downstream VEGFR-3 autophosphorylation as a result of neuropilin sequestering of VEGF-C can be determined in this more biologically relevant situation.

The above experiments will also be carried out with relevant semaphorin proteins in conjunction with the neuropilin composition of the invention to determine the effects of another natural ligand for the neuropilin receptor on blocking VEGF-C/neuropilin receptor interactions. If VEGF-C and semaphorin bind neuropilins in the same site on the receptor, there will be a subsequent increase in VEGF-C binding to VEGFR-3 and VEGFR-3 phosphorylation, due to the increase in VEGF-C unbound to the neuropilin-Fc. However, if the semaphorins and VEGF-C bind at different sites on the neuropilin receptor and do not inhibit each other's binding, then the amount of VEGF-C binding to VEGFR-3 will be comparable to binding in the absence of the semaphorins, i.e. with neuropilin-Fc alone. This assay will further define VEGF-C/neuropilin interactions.

The aforementioned in vitro cell-free and cell-based assays can also be performed with putative modulator compounds, e.g. cytokines that affect VEGF-C secretion (TNFa, TGFb, PDGF, TGFa, FGF-4, EGF. IL-1a IL-1b, IL-6) to determine the efficacy of the neuropilin composition at blocking VEGF-C activity in the presence of VEGF-C modulators which are biologically active in situations of inflammation and tumor growth, comparing the neuropilin composition to current experimental cancer therapeutics.

### EXAMPLE 4

### Effects of Neuropilin-2/VEGF-C Binding on VEGF-C Related BIOLOGICAL FUNCTIONS

VEGF-C is intimately involved with many functions of lymphangiogenesis and endothelial cell growth. The influence of NRP-2 on such VEGF-C functions in vivo is investigated using the following assays:

A. Cell Migration Assay

For example, human microvascular endothelial cells (HMVEC) express VEGFR-3 and NRP-2, and such cells can be used to investigate the effect of soluble and membrane bound neuropilin receptors on such cells. Since neuropilins and VEGF/VEGFR interactions are thought to play a role in migration of cells, a cell migration assay using HMVEC or other suitable cells can be used to demonstrate stimulatory or inhibitory effects of neuropilin molecules.

Using a modified Boyden chamber assay, polycarbonate filter wells (Transwell, Costar, 8 micrometer pore) are coated with 50 μg/ml fibronectin (Sigma), 0.1% gelatin in PBS for 30 minutes at room temperature, followed by equilibration into DMEM/0.1% BSA at 37° C. for 1 hour. HMVEC (passage 4-9, 1×10⁵ cells) naturally expressing VEGFR-3 and neuropilin receptors or endothelial cell lines recombinantly expressing VEGFR-3 and/or NRP-2 are plated in the upper chamber of the filter well and allowed to migrate to the undersides of the filters, toward the bottom chamber of the well, which contains serum-free media supplemented with prepro-VEGF-C, or enzymatically processed VEGF-C, in the presence of varying concentrations of neuropilin-1-Fc, neuropilin-2-Fc, and VEGFR-3-Fc protein. After 5 hours, cells adhering to the top of the transwell are removed with a cotton swab, and the cells that migrate to the underside of the filter are fixed and stained. For quantification of cell numbers, 6 randomly selected 400× microscope fields are counted per filter.

In another variation, the migration assay described above is carried out using porcine aortic endothelial cells (PAEC) stably transfected with constructs such as those described previously, to express NRP-2, VEGFR-3, or both NRP-2 and VEGFR-3 (i.e. PAE/NRP-2, PAE/VEGFR-3, or PAE/NRP-2/VEGFR-3). PAEC are transfected using the method described in Soker et al. (Cell 92:735-745. 1998). Transfected PAEC (1.5×10⁴ cells in serum free F12 media supplemented with 0.1% BSA) are plated in the upper wells of a Boyden chamber prepared with fibronectin as described above. Increasing concentrations of VEGF-C or VEGF-D are added to the wells of the lower chamber to induce migration of the endothelial cells. After 4 hrs, the number of cells migrating through the filter is quantitated by phase microscopy.

An increase in migration and chemotaxis of NRP-2/VEGFR-3 double transfectants over NRP-2 or VEGFR-3 single transfectants indicates that the presence of neuropilin-2 enhances the ability of VEGF-C or VEGF-D to signal through VEGFR-3 and stimulate downstream biological effects, particularly cell migration and, likely, angiogenesis or lymphangiogenesis.

Additionally, the porcine aortic endothelial cell migration assay is used to identify modulators of NRP-2NEGFR-3/VEGF-C mediated stimulation of endothelial cells. Migration of PAE/NRP-2/VEGFR-3 expressing cells is assessed after the addition of compositions, such as soluble receptor peptides, proteins or other small molecules (e.g. monoclonal and bispecific antibodies or chemical compounds), to the lower wells of the Boyden chamber in combination with VEGF-C ligand. A decrease in migration as a result of the addition of any of the peptides, proteins or small molecules identifies that composition as an inhibitor of NRP-2/VEGFR-3 mediated chemotaxis.

B. Mitogen Assay

Embryonic endothelial cells expressing VEGFR-3 alone, NRP-2 alone, or both VEGFR-3 and NRP-2 are cultured in the presence or absence of VEGF-C polypeptides, and potential modulators of this interactions such as semaphorins, more particularly Sema3F, as well as cytokines which may include but are not limited to TGF-β, TNF-α, IL-1α and IL-1β, IL-6, and PDGF, known to upregulate VEGF-C activity, to assay effects on cell growth using any cell growth or migration assay, such as assays that measure increase in cell number or assays that measure tritiated thymidine incorporation. See, e.g., Thompson et al., Am. J. Physiol. Heart Circ. Physiol., 281: H396-403 (2001).

### EXAMPLE 5

### Angiogenesis Assays

There continues to be a long-felt need for additional agents that can stimulate angiogenesis, e.g., to promote wound healing, or to promote successful tissue grafting and transplantation, as well as agents to inhibit angiogenesis (e.g., to inhibit growth of tumors). Moreover, various angiogenesis stimulators and inhibitors may work in concert through the same or different receptors, and on different portions of the circulatory system (e.g., arteries or veins or capillaries; vascular or lymphatic). Angiogenesis assays are employed to measure the effects of neuropilin/VEGF-C interactions, on angiogenic processes, alone or in combination with other angiogenic and anti-angiogenic factors to determine preferred combination therapy involving neuropilins and other modulators. Exemplary procedures include the following.

A. In Vitro Assays for Angiogenesis

1. Sprouting Assay

HMVEC cells (passage 5-9) are grown to confluency on collagen coated beads (Pharmacia) for 5-7 days. The beads are plated in a gel matrix containing 5.5 mg/ml fibronectin (Sigma), 2 units/ml thrombin (Sigma), DMEM/2% fetal bovine serum (FBS) and the following test and control proteins: 20 ng/ml VEGF, 20 ng/ml VEGF-C, or growth factors plus 10 micrograms/ml neuropilin-2-Fc, and several combinations of angiogenic factors and Fc fusion proteins. Serum free media supplemented with test and control proteins is added to the gel matrix every 2 days and the number of endothelial cell sprouts exceeding bead length are counted and evaluated.

2. Migration Assay

The transwell migration assay previously described may also be used in conjunction with the sprouting assay to determine the effects the neuropilin compositions of the invention have on the interactions of VEGF-C activators and cellular function. The effects of VEGF-Cs on cellular migration are assayed in response the neuropilin compositions of the invention, or in combination with known angiogenic or anti-angiogenic agents. A decrease in cellular migration due to the presence of the neuropilins after VEGF-C stimulation indicates that the invention provides a method for inhibiting angiogeneis.

This assay may also be carried out with cells that naturally express either VEGFR-3 or VEGFR-2, e.g. bovine endothelial cells which preferentially express VEGFR-2. Use of naturally occurring or transiently expressing cells displaying a specific receptor may determine that the neuropilin composition of the invention may be used to preferentially treat diseases involving aberrant activity of either VEGFR-3 or VEGFR-2.

B. In Vivo Assays for Angiogenesis

1. Chorioallantoic Membrane (CAM) Assay

Three-day old fertilized white Leghorn eggs are cracked, and chicken embryos with intact yolks are carefully placed in 20×100 mm plastic Petri dishes. After six days of incubation in 3% CO₂ at 37 degrees C., a disk of methylcellulose containing VEGF-C and various combinations of the neuropilin compositions, VEGFR-3, and neuropilin-2 and VEGFR-3 complexes, dried on a nylon mesh (3×3 mm) is implanted on the CAM of individual embryos, to determine the influence of neuropilins on vascular development and potential uses thereof to promote or inhibit vascular formation. The nylon mesh disks are made by desiccation of 10 microliters of 0.45% methylcellulose (in H₂O). After 4-5 days of incubation, embryos and CAMs are examined for the formation of new blood vessels and lymphatic vessels in the field of the implanted disks by a stereoscope. Disks of methylcellulose containing PBS are used as negative controls. Antibodies that recognize both blood and lymphatic vessel cell surface molecules are used to further characterize the vessels.

2. Corneal Assay

Corneal micropockets are created with a modified von Graefe cataract knife in both eyes of male 5- to 6-week-old C57BL6/J mice. A micropellet (0.35×0.35 mm) of sucrose aluminum sulfate (Bukh Meditec. Copenhagen. Denmark) coated with hydron polymer type NCC (IFN Science. New Brunswick, N.J.) containing various concentrations of VEGF molecules (especially VEGF-C or VEGF-D) alone or in combination with: i) factors known to modulate vessel growth (e.g. 160 ng of VEGF, or 80 ng of FGF-2); ii) neuropilin polypeptides outlined above; or iii) neuropilin polypeptides in conjunction with natural neuropilin ligands such as semaphorins, e.g. Sema-3C and Sema3F, is implanted into each pocket. The pellet is positioned 0.6-0.8 mm from the limbus. After implantation, erythromycin/ophthamic ointment is applied to the eyes. Eyes are examined by a slit-lamp biomicroscope over, a course of 3-12 days. Vessel length and clock-hours of circumferential neovascularization and lymphangiogenesis are measured. Furthermore, eyes are cut into sections and are immunostained for blood vessel and/or lymphatic markers (LYVE-1 [Prevo et al., J. Biol. Chem., 276: 19420-19430 (2001)], podoplanin [Breiteneder-Geleffet et al., Am. J. Pathol., 154: 385-94 (1999).] and VEGFR-3) to further characterize affected vessels.

### EXAMPLE 6

### In Vivo Tumor Models

There is mounting evidence that neuropilin receptors may play a significant role in tumor progression. Neuropilin-1 receptors are found in several tumor cell lines and transfection of NRP-1 into AT2.1 cells can promote tumor growth and vascularization (Miao et al, FASEB J. 14: 2532-39.2000). Additionally, investigation of neuropilin-2 expression in carcinoid tumors, slowly developing tumors derived from neuroendocrine cells in the digestive tract, illustrates that neuropilin-2 is actually expressed in normal tissue surrounding the tumor, but not in the center of the tumor itself (Cohen et al, Biochem. Biophys. Res. Comm. 284: 395-403. 2001), and it is established that neuroendocrine cells secrete VEGF-C, VEGF-D, and express VEGFR-3 on their cell surface (Partanen et al., FASEB J 14:2087-96. 2000). Differential expression levels of these neuropilins in association with VEGF molecules, which are often correlative with vascular density and tumor progression, in and around tumors could be indicative of tumor progression or regression.

A. Ectopic Tumor Implantation

Six- to 8-week-old nude (nu/nu) mice (SLC. Shizuoka. Japan) undergo subcutaneous transplantation of C6 rat glioblastoma cells or PC-3 prostate cancer cells in 0.1 mL phosphate-buffered saline (PBS) on the right flank. The neuropilin polypeptides outlined previously are administered to the animals at various concentrations and dosing regimens. Tumor size is measured in 2 dimensions, and tumor volume is calculated using the formula, width2× length/2. After 14 days, the mice are humanely killed and autopsied to evaluate the quantity and physiology of tumor vasculature in response to VEGF-C inhibition by neuropilin polypeptides.

It will be apparent that the assay can also be performed using other tumor cell lines implanted in nude mice or other mouse strains. Use of wild type mice implanted with LLC lung cancer cells and B16 melanoma cells is specifically contemplated.

B Orthotopic Tumor Implantation

Approximately 1×10⁷ MCF-7 breast cancer cells in PBS are inoculated into the fat pads of the second (axillar) mammary gland of ovarectomized SCID mice or nude mice, carrying s.c. 60-day slow-release pellets containing 0.72 mg of 17β-estradiol (Innovative Research of America). The ovarectomy and implantation of the pellets are done 4-8 days before tumor cell inoculation. The neuropilin polypeptides and VEGF-C polypeptides outlined previously, as well as semaphorins, specifically Sema3C and Sema3F, are administered to the animals at various concentrations and dosing regimens. Tumor size is measured in 2 dimensions, and tumor volume is calculated using the formula, width 2× length/2. After 14 days, the mice are humanely killed and autopsied to evaluate the quantity and physiology of tumor vasculature.

A similar protocol is employed wherein PC-3 cells are implanted into the prostate of male mice.

C. Lymphatic Metastasis Model

VEGF-C/VEGFR3 interactions are often associated in adult tissue with the organization and growth of lymphatic vessels, thus the presence of neuropilin receptor at these sites may be involved in the metastatic nature of some cancers. The following protocol indicates the ability of neuropilin polypeptides, especially neuropilin-2 polypeptides, or fragments thereof for inhibition of lymphatic metastasis.

MDA-MB-435 breast cancer cells are injected bilaterally into the second mammary fat pads of athymic, female, eight week old nude mice. The cells often metastasize to lymph node by 12 weeks. Initially, the role of neuropilin-2 binding to VEGF-C and VEGFR-3 in tumor metastasis can be assessed using modulators of neuropilin-VEGF-C binding determined previously, especially contemplated are the semaphorins. A decrease in metastasis correlating with NRP-2 blockade indicates NRP-2 is critical in tumor metastasis. The modulators of neuropilin-VEGF-C binding determined previously [by the invention] are then administered to the animals at various concentrations and dosing regimens. Moreover, the neuropilin-2 polypeptides are administered in combination with other materials for reducing tumor metastasis. See, e.g., International Patent Publication No. WO 00/21560, incorporated herein by reference in its entirety. Mice are sacrificed after 12 weeks and lymph nodes are investigated by histologic analysis. Decrease in lymphatic vessels and tumor spread as a result of administration of the neuropilin compositions indicate the invention may be a therapeutic compound in the prevention of tumor metastasis.

### EXAMPLE 7

### Assessment of VEGF-C on Growth Cone Collapse by Collagen Repulsion Assay

The constitutive expression of semaphorins in the central nervous system has been proposed as a primary factor in the lack of regeneration of nerves in this area. Regeneration of peripheral nerves after nerve insult, such as sciatic nerve crush, is made possible by the downregulation of semaphorin-3A expression immediately following injury. Sema3A expression returns to baseline levels after approximately 36 days following injury, but this extended period of decreased semaphorin expression allows for the growth and regeneration of the peripheral nerve into the area of damage before the regrowth is halted by semaphorin activity (reviewed in Pasterkamp and Verhaagen, Brain Res. Rev. 35: 36-54. 2000). While numerous semaphorins are extensively expressed in the CNS and PNS, semaphorin-3F, the primary ligand for neuropilin-2, demonstrates wide distribution in human brain, and has even been found to be overexpressed in certain areas of the brain in Alzheimer's patients (Hirsch et al, Brain Res. 823:67-79. 1999). The newly discovered interaction of VEGF-C binding to NRP-2 may provide a factor for specifically inhibitino the actions of sema-3F activity in halting neural regeneration in many neurodegenerative diseases such as Alzheimer's or macular degeneration. Moreover, the apparent neurotrophic effects of VEGF-C (described in Example 8, for example) may synergistically combine with a sema-3F-inhibitory activity to produce beneficial results.

Superior cervical ganglia (SCG) are dissected out of E13.5 or E15.5-17.5 rat or mouse embryos according to the method of Chen et al (Neuron, 25:43-56. 2000) and Giger et al (Neuron, 25:29-41.2000) for use in a collagen repulsion assay. Following dissection, hindbrain-midbrain junction explants are co-cultured with COS cells recombinantly modified to express Alkaline phosphatase conjugated Sema3F or mock transfected COS cells in collagen matrices in culture medium [OPTI-MEM and F12 at 70:25, supplemented with 1% P/S, Glutamax (Gibco), 5% FCS and 40 mM glucose] for 48 h. Neurite extension is quantitated using the protocol outlined by Giger et al (Neuron, 25:29-41. 2000), briefly described by determining the percentage of neurite extension beyond a defined point in the culture matrix. Neurite extension can be measured in the presence of varying concentrations of a VEGF-C composition as compared to in the absence of a VEGF-C composition and the subsequent increase of neurite extension as a result of VEGF-C addition to the culture and blockade of Sema3F interaction with neuropilin-2 can be assessed.

The effects of Sema3F inhibition as a result of the present invention may be extrapolated into treatments for several diseases wherein neuronal regeneration is prohibited by the presence of semaphorins, for example scarring after cranial nerve damage, and perhaps in the brains of Alzheimer's patients.

Variations to the examples above will be apparent and are considered aspects of the invention within the claims. For example, the materials and methods described in the preceding Examples are useful and readily adapted for screening for new modulators of the polypeptide interactions described herein, and for demonstrating the effects of such new modulators in cell-based systems and in vivo. In other words, the procedures in the materials and methods of the Examples are useful for identifying modulators and screening the modulators for activity in vitro and in vivo.

By way of illustration. Example 1 describes an experimental protocol wherein VEGF-C binding to neuropilins was investigated. Similar binding experiments can be performed in which a test agent is added to the binding experiment at one or more test agent concentrations, to determine if the test agent modulates (increases or decreases) the measurable binding between VEGF-C and the neuropilin. Example 2 describes an experimental protocol wherein VEGFR-3 binding to neuropilins was investigated. Similar binding experiments can be performed in which a test agent is included in the reaction to determine if the test agent modulates (increases or decreases) the measurable binding between VEGFR-3 and the neuropilin. Test agents that are identified as modulators in initial binding assays can be included in cell-based and in vivo assays that are provided in subsequent Examples, to measure the biological effects of the test agents on cells that express receptors of interest (e.g., VEGFR-3 or neuropilin-expressing cells) or on biological systems and organisms.

Similarly, a number of the Examples describe using a soluble form of neuropilin receptor or other protein in experiments that further prove binding relationships between molecules described herein for the first time. These experiments also demonstrate that molecules that bind one or both members of a ligand/receptor pair or receptor/co-receptor pair can be added to a system to modulate (especially inhibit) the ability of the binding pair to interact. For example, soluble NRP molecules are used in Example 3 to modulate (inhibit) VEGF-C or VEGF-D binding to VEGFR-3 or VEGFR-2. The disruption of VEGF-C or VEGF-D binding to their respective VEGFR receptors has practical applications for treatment of numerous diseases characterized by undesirable ligand-mediated stimulation of VEGFR-3 or VEGFR-2. Similar binding experiments can be performed in which a test agent suspected of modulating the same binding reactions is substituted for the soluble NRP molecule. In this way, the materials and methods of the Examples are used to identify and verify the therapeutic value of test agents.

### EXAMPLE 8

### Phenotype of VEGF-C−/− Animals

In order to analyze the role of VEGF-C in lymphangiogenesis and neuronal growth, mice deficient in the VEGF-C gene were generated by replacing the VEGF-C first coding exon with the LacZ gene.

A. Generation of VEGF-C Knockout Mice:

The VEGF-C gene was isolated from a 129Sv mouse genomic library in 5′ and 3′ segments. A 2.9-kb BamHI-PstI fragment was blunt-end cloned into the BamHI site of the pNTP1oxP targeting vector to make the 3′ arm. The 3.3-kb 5′ arm was excised by HindIII and (partial) BsmBI digestion and inserted into the pSDKlacZ plasmid upstream of the LacZ/NeoR block. Subsequently, a SalI cassette of this construct was cloned into the XhoI site of the pNTP1oxP plasmid containing the 3′arm to generate the final targeting vector. The 5′arm was designed to delete the first exon, including a 125-bp fragment upstream of the translation initiation site, the first 147-bp (49 codons) of the coding region and 143-bp of the first intron (including the signal peptide). This placed the LacZ reporter gene under the control of the regulatory regions of the VEGF-C gene.

The targeting construct was electroporated into R1 (129/Sv×129/SvJ) mouse ES cells. Screening for the targeted mutation was done by Southern blot analysis using NcoI digestion and a 5′ external probe. Positive clones were aggregated with WT morulas to obtain chimeric mice, which were bred with ICR mice. The pups were genotyped by Southern blotting or by PCR using primers 5′-TCC GGT TTC CTG TGA GGC-3′ (forward) (SEQ ID NO: 34), 5′-AAG TTG GGT AAC GCC AGG-3′ (reverse for targeted allele) (SEQ ID NO: 35) and 5′-TGA CCT CGC CCC CGT C-3′ (reverse for VEGF-C 1st exon) (SEQ ID NO: 36).

B. Lethality of VEGF-C−/− Phenotype

Only a few VEGF-C−/− pups were found among 243 offspring of VEGF-C+/− mice, suggesting that VEGF-C deficiency results in embryonic lethality. The VEGF-C−/− embryos were found at the expected frequency but most of them were edematous from E12.5 onwards and severely swollen and growth retarded at E18.5. All VEGF-C−/− embryos died late.

Whole mount staining for β-galactosidase activity in embryos containing the LacZ-VEGF-C marker gene indicated that VEGF-C was strongly expressed from E8.5 onwards in the jugular region where the first ivmph sacs form (Kukk et al., Development 122, 3829. 1996). Accordingly, double staining for β-galactosidase and VEGFR-3 in sections of E10.5 VEGF-C+/−embryos indicated that VEGF-C is abundant in the mesenchyme dorso-lateral to the VEGFR-3 positive jugular veins, which give rise to the lymphatic endothelium.

The localization and timing of VEGF-C expression suggested that VEGF-C plays a role in the development of the lymphatic vasculature. Accordingly, staining of sections from the jugular region for the lymphatic markers VEGFR-3, LYVE-1 or podoplanin showed that the lymph sacs did not form in the VEGF-C−/−embryos, whereas they were clearly visible in their VEGF-C+/−and VEGF-C+/+littermates. Interestingly, VEGFR-3 expression also continued in some erythrocyte-containing capillaries of the VEGF-C−/− embryos whereas it was downregulated in their littermates. The veins and arteries appeared normal in PECAM-1 and smooth muscle actin stained sections. VEGFR-3 whole mount staining of the VEGF-C−/−embryos at E17.5 indicated that at later stages the lymphatic vessels including the thoracic duct were also absent.

C. Prox-1 Expression in VEGF-C−/− Embryos

Prox-1 is a transcription factor expressed in lymphatic endothelial cells which is useful in measuring the extent of lymphatic network formation. Similar to VEGF-C−/− embryos, embryos deficient in Prox-1 also fail to form the primitive lymph sacs (Wigle and Oliver, Cell 98, 769 (1999) Wigle et al., EMBO J. 21, 1505 (2002)). To measure the effects of VEGF-C expression on Prox-1, Prox-1 expression was studied in VEGF-C−/− embryos by whole mount immunofluorescence.

To produce Prox-1 antibodies, cDNA encoding Prox-1 (SEQ ID NO: 37) homeobox domain and prospero domain (amino acids 578-750 of human Prox-1, SEQ ID NO: 38) was subcloned into the pGEX2t vector to produce a GST-Prox-1 fusion construct, and the GST-Prox-1 fusion protein was purified from E. coli using glutathione Sepharose according to the manufacturer's instructions (Amersham, Piscataway, N.J.). The fusion protein was used to immunize rabbits according to a standard protocol, and Prox-1 specific antibodies were isolated from rabbit serum using sequential columns with GST- and GST-Prox-1-coupled to vinylsulfone agarose resin (Sigma). The purified antibody recognized an 85-kD protein in lysates from 293T cells transfected with Prox-1, but not from cells transfected with the empty vector. The antibodies also specifically stained lymphatic but not blood endothelial cells in frozen sections of mouse skin.

For the whole mount explants, the axial vascular system, part of the endodermal, and all intermediate mesodermal derivatives from E10-E13 embryos were separated. At E10.5, strong endothelial Prox-1 staining was detected bilaterally in the jugular veins in all embryos. These Prox-1 expressing lymphatic endothelial cells had started sprouting in the VEGF-C+/+ and in the VEGF-C+/−embryos, whereas the Prox-1 expressing endothelial cells in the VEGF-C−/− embryos were confined to the wall of the cardinal vein. Subsequently, the Prox-1 expressing endothelial cells in the VEGF-C+/+ and in the VEGF-C+/−embryos formed the jugular lymph sacs, which were clearly seen at E13. However, in the VEGF-C−/− embryos, there were only a few Prox-1 expressing endothelial cells left in the cardinal vein at this stage and no lymph sac like structures were found. Prox-1 expression in cardiomyocytes and hepatocytes appeared normal in the VEGF-C−/− embryos at all stages analyzed. This suggested that VEGF-C is not needed for cell commitment to the lymphatic endothelial lineage, but that paracrine VEGF-C signaling is required for the migration of the Prox-1 expressing endothelial cells from the cardinal vein and for the subsequent formation of the lymph sacs. In the absence of VEGF-C, the number of Prox-1 expressing endothelial cells also decreased by E13, suggesting that VEGF-C is required for the survival of these cells.

D. VEGF-C Expression in the Nervous System

Analysis of VEGF-C expression in regions of VEGF-C−/− embryonic development aside from lymphatic development indicated that VEGF-C expression during embryogenesis was also localized to the nervous system. Analysis of Prox-1 expression in the VEGF-C−/− mice also demonstrated that Prox-1 co-localized with VEGF-C in the mid-hindbrain region, and was also expressed in the developing eye and in the region of the developing forelimb. No Prox-1 expression was detected in the mid-hindbrain region in VEGF-C−/− embryos while levels remained the same at other sites in VEGF-C−/− animals.

VEGF-C was strongly expressed in the mid-hindbrain region and in the wall of the cerebellum at various stages of embryogenesis. VEGF-C expression in adult brains was detected via in situ hybridization of VEGF-C+/−animals. VEGF-C was detected the majority of brain regions in the adult animal, including the cerebellum (granular and purkinje cells), smooth muscle cells in the brain, the subventricular zone (SVZ), olfactory bulb glial cells, hypothalamus, hippocampus, brain stem, the visual zone, regions of the cerebral cortex, and the cranial ganglias.

The extensive VEGF-C expression in the brain suggests that it has a role in the CNS. VEGF-C may function as neuroprotective or neurotrophic agent in the CNS. In addition, its expression in the smooth muscle cells surrounding the blood vessels suggests that VEGF-C may have a function (eg. survival or permeability function) on the endothelial cells in the brain. The expression in the visual zone suggests that VEGF-C may have a crucial function in the development and maintenance of the visual system. Furthermore, the SVZ is known to contain neural progenitors (Picard-Riera et al., Proc. Natl. Acad. Sci. USA 99:13211-13216.2002). From this zone, the progenitors migrate through the rostral migratory stream to the olfactory bulb, where they replace the periglomerular and granular neurons. However, the SVZ cells can be triggered to proliferate more extensively and to differentiate into astrocytes in response to injury (Picard-Riera et al., supra). Thus, VEGF-C may play a role in the survival and proliferation and/or migration of the neural progenitor cells.

D.1 VEGF-C Induces Proliferation of Prox-1 Positive Cells

The effects of exogenous VEGF-C were analyzed in tissue explants from the VEGF-C−/− and VEGF-C+/+ embryos on embryonic day (E) 11.5, using VEGF-C release from agarose beads. Affi-Gel Blue beads (mesh size 100-200; Bio-Rad, Hercules, Calif.) were incubated in PBS containing 100 ng/μl of VEGF-C (Pichia pastoris produced hVEGF-C ΔNΔC-6×His, described in (Joukov et al., 1997)). In control samples, 100 ng/μl human serum albumin (HSA); or 1% BSA containing agarose beads were used. The beads were added to the tissue explant as follows: two beads lateral from dorsal aorta close to the metanephric region, two beads lateral from the dorsal aorta to the cranial mesonephric region and two beads lateral from the aortic arches to the jugular region. The explants were cultured for 48 hours on Track-tech Nuclepore filters (pore-size 0.1 μm; Whatmann) placed on top of a metal grid in Trowell-type organ culture system (Sainio, 2003).

After 48 hours in culture, the embryos were fixed and analyzed for Prox-1 and PECAM-1 expression by immunohistochemistry. For immunohistochemical staining, the tissues were fixed in −20° C. methanol for 10 min, washed with PBS three times and blocked with 1% BSA in PBS at 4° C. for 1 hour. The tissues were then incubated overnight in the primary antibodies diluted in blocking solution. The primary antibodies used were rat-anti-mouse PECAM-1 (PharMingen. San Diego, Calif.), and affinity-purified rabbit-anti-Proxl. Cy2, FITC or TRITC-1 labeled secondary antibodies (Jackson Laboratories) were used for staining. The tissues were mounted with Immu-mount™ (Thermo Shandon, Pittsburgh, Pa.) or with Vectashield (Vector Laboratories) and analyzed by Zeiss Axioplan 2 fluorescent microscope.

In general, the high concentrations of VEGF-C used destroyed the normal arterial/venous hierarchy of the vessels. In all embryos, Prox-1/PECAM-1 expressing lymphatic endothelial cells migrated towards the VEGF-C expressing beads. However, in all genotypes, VEGF-C also induced massive proliferation of Prox-1 positive and PECAM-1 negative cells. As all other Prox-1 expressing cells/tissues (e.g. liver primordia, heart, dorsal ganglia; see (Oliver et al., Mech Dev. 44:3-16. 1993) had been dissected out from the tissue preparations, these cells must have originated from the developing sympathetic neural system (sympathetic ganglia), in which Prox-1 has been shown to be expressed (Wigle et al., EMBO J. 21:1505-1513. 2002).

### EXAMPLE 9

### VEGF-C and Differentiation of Sympathetic Ganglia

A. Effects of VEGF-C or VEGF-D on Neuronal Expansion

In order to analyze the neural cell populations in more detail, sympathetic ganglia from the embryo explants were isolated and cultured. E11 wild-type (NMRI mouse) embryos were dissected and a VEGF-C bead experiment was performed as above using VEGF-C ΔNΔC. Beads containing BSA were used as a control.

E11.5 embryos from the VEGF-C knockout mouse or E11 mouse (NMR1) wild-type embryos were dissected as follows: from the retroperitoneal area the urogenital tissues with gonads, mesonephric and metanephric kidney primordia were dissected (Sainio. 2003). Intestine, liver primordia, heart and lung primordia were removed. The dorsal aorta and the sympathetic ganglia chain in its ventrolateral sides were left intact. In the jugular area, the aortic arches and the sympathetic chain were also left intact.

After 48 hours, the sympathetic ganglia of wild-type mice had formed a clearly transparent and expanded area around the VEGF-C beads, and were removed and mechanically dissociated. Two of the VEGF-C bead-containing NMRI explants were removed from the filters to the standard, freshly made culture media (D-MEM F12 (3:1) supplemented with B27) containing EGF (20 ng/ml) and FGF (40 ng/ml) to support the survival and proliferation of undifferentiated neurons. VEGF-C (100 ng/ml) was added to the medium and the pieces were cultured at 37° C. After 72 hours, there were clear neurospheres in the cultures. These neurospheres were then collected and cultured in neural stem cell medium (DMEM/F12 described above) containing VEGF-C (100 ng/ml), or plated on media without EGF and FGF, thus allowing the differentiation of the neurons.

For differentiation assays, four of the VEGF-C bead-containing NMRI explants and the control (BSA bead-containing) explants are fixed after 48 hours in culture with ice-cold methanol and are processed for whole-mount immunohistochemistry. Alternatively, to detect cellular differentiation, neurospheres are dissociated and plated as single cells on a polylysin-coated cover slip in 24-well plate well in EGF-FGF free medium supplemented with 100 ng/ml nerve growth factor (NGF) for 4 days. Antibodies that detect the primary neurons (Tuj-1 and p75 NGF-receptor), epithelial structures (pan-cytokeratin) and differentiated neurons (tyrosine hydroxylase (TH), neurofilament antibodies) are used to confirm that it is the sympathetic neural cells that proliferate in these cultures and to determine VEGF-C influence on neural differentiation.

B. Effects of VEGF-C or VEGF-D on Neurite and Axonal Outgrowth

The above experiments indicate that VEGF-C acts as a neurotrophic growth factor. To determine the effects of VEGF-C or VEGF-D products on proliferation or regeneration of adult axons, axonal outgrowth assays are performed in the presence and absence of VEGF-C and VEGF-D products with or without culture with other neurotrophic factors.

For example, superior cervical ganglia (SCG) are dissected from adult rats and mounted in MATRIGEL® as in Sondell et al (J. Neurosci. 19:5731-40. 1999). Two to three ganglia are mounted per 35 mm culture dish and explant cultures are maintained in RPMI 1640 serum free medium in a humidified chamber of 5% CO₂ for 48 hours or 72 hours. VEGF-C product or VEGF-D product is added to the culture at varying timepoints post mounting, including at 0 hours. 4 hours. 6 hours, 8 hours, 12 hours, or 24 hours after explant. VEGF-C or VEGF-D is added over dose ranges from ng/ml to μg/ml, such as 1, 10, 25, 50, 100 or 200 ng/ml. Nerve growth factor is used as a positive control while non-treated ganglia or ganglia treated with irrelevant protein are used as a negative control.

To measure the extent of axonal growth induced by VEGF-C or VEGF-D products, both the length and density of axons grown in culture are measured. Increased axon length and axon density in the VEGF-C or VEGF-D treated ganglia indicates that VEGF-C or VEGF-D induces adult axons to grow and may be useful therapies for axonal growth in human neuropathologies requiring axonal regeneration.

Additional experiments are carried out to measure the synergistic effects of treating axonal explants with VEGF-C or VEGF-D in combination with other neurotrophic factors.

The effects of VEGF-C and VEGF-D are further assessed on embryonic axons. Trigeminal ganglia are dissected from E10-E12 rat embryos and embedded into three-dimensional collagen matrix prepared according to Ebendal (1989). Typically, 3-5 ganglia are cultured in 0.5 ml of matrix in 24-well tissue culture plates. The gels are covered by 0.5 ml of Eagle's Basal Medium (GIBCO BRL) containing 1% heat-inactivated horse serum. The collagen gel is prepared into the same medium. Recombinant VEGF-C or VEGF-D products are added to the culture media and control cultures are devoid of any factors, NGF cultures can serve as positive control. The neurotrophic factors are typically applied at ng/ml or μg/ml concentrations, e.g. 1, 10, 25, 50, 100 or 200 ng/ml. The explant cultures are incubated at 37° C. in a humidified atmosphere containing 5% CO₂ in the presence or absence of VEGF-C product or VEGF-D product and examined after 24 and 48 hours for neurite outgrowth and optionally stained with anti-neurofilament antibodies to better visualize the neurites.

C. Neurotrophic Effects of VEGF-C or VEGF-D in a Model of Spinal Cord Injury

A major requirement in the treatment of nerve trauma or injury is the regeneration of axons at the site of injury. To assess the neurotrophic effects of VEGF-C and VEGF-D products in stimulating axon regeneration, a rat model of spinal cord injury is used. For instance, adult rats are transected at the T-8 level of the spinal cord according to Facchiano et al. (J. Neurosurg. 97:161-68. 2002) and administered, at the site of lesion. VEGF-C or VEGF-D products suspended in matrigel which allows for a slow release of the therapeutic. Animals may also be administered VEGF-C or VEGF-D products via other well-established treatment routes such as intraperitoneal, intravenous, or retro orbital injection. Administration systemically is an option, but local administration at the site of injury is preferred. VEGF-C or VEGF-D product is administered in doses pre-determined to be effective for the size and type of animal being treated, and may be administered in one treatment or over a course of treatments, such as every 2 days, once weekly or any other regimen effective for the animal being treated. Control animals receive either no treatment or treatment with irrelevant protein such as bovine serum albumin.

To assess the extent of axon regeneration in the VEGF-C- or VEGF-D-treated animals, the spinal cord is dissected out at varying timepoints after treatment, e.g. day 14, day 21 or day 28 after initial spinal cord transection and degeneration of the axons measured according to the methods of Facchiano et al. (supra), wherein the distance between transection site and tips of the new axons are measured, indicating whether or not the axons grow in response to growth factor or if they cannot respond and simply die.

An increase in axon regeneration in the VEGF-C or VEGF-D treated animals as compared to control animals indicates that VEGF-C or VEGF-D acts as a potent neurotrophic factor and promotes axonal regeneration critical to repairing motor neuron injury.

To characterize VEGF-C or VEGF-D receptor expression in the sympathetic or motor neurons in the experiments described above, isolated neuronal cells (both before and after VEGF-C or VEGF-D stimulation) are stained with antibodies directed to VEGFR-2, VEGFR-3, NRP-1 and NRP-2.

### EXAMPLE 10

### Proliferation of Neurons in the Presence of VEGF-C or VEGF-D

To quantify the mitogenic potential of VEGF-C or VEGF-D products in cultures of sympathetic neurons, proliferation (MTT) assays are performed.

The neurospheres cultured in neuronal cell medium are stimulated with VEGF-C, VEGF-D, VEGF-C ΔC₁₅₆, or other forms of VEFG-C or VEGF-D product. VEGF (or another growth factor) or with control proteins for 48 hours in starvation medium (w/o serum). Cells are incubated with the MTT substrate, 3-[4,5-dimethylthiazol-2-y]-2,5-diphenyltetrazolium bromide, (5 mg/ml) for 4 hours at 37° C., lysed and the optical density at 540 nm is measured.

Additionally, VEGF-C or VEGF-D product is tested for the ability to stimulate cell proliferation using Bromodeoxyuridine (BrdU) incorporation and/or tritiated thymidine incorporation as a labeling index and as a measure of cell proliferation [Vicario-Abejon et al., Neuron 15:105-114 (1995)]. For example, neuronal cells are plated and then pulsed with BrdU for a set amount of time (e.g., 18 hours) in the presence or absence of VEGF-C or control protein, prior to fixation. The cells are fixed and neutralized, and incubated with BrdU monoclonal antibody. The BrdU antibody is then detected with a labeled secondary antibody. To examine if BrdU-positive cells are of a specific subset of neuron, BrdU labeling is combined with staining for neuron-specific markers as set forth above.

Neuronal proliferation is also measured in vivo by a non-invasive method by measuring neuron density by NMR microscopy (See U.S. Pat. No. 6,245,965). Additionally, animals models and controls can be administered BrdU or tritiated thymidine prior to, during, and/or after the administration of VEGF-C: After the final injection, the animals are anesthetized and/or sacrificed, and the tissues of interest are removed. These tissues are analyzed as for BrdU incorporation using anti-Brdu antibodies, or by measuring the amount of [³H] counts in cell extracts.

Fragments and analogs of VEGF-C and VEGF-D polypeptides are used in the above proliferation assays to determine the minimal VEGF-C fragments useful in mediating neural stem cell growth and differentiation. Delineation of a minimal VEGF-C or VEGF-D polypeptide fragment capable of stimulating neural stem cell growth may provide a VEGF-C or VEGF-D polypeptide small enough to transverse the blood brain barrier. Development of a therapeutic which flows across the blood brain barrier could eliminate invasive methods of administration of VEGF-C or VEGF-D polypeptides and lead to more moderate forms of treatment such as intravenous or subcutaneous injections.

### EXAMPLE 11

### VEGF-C- or VEGF-D-Expressing Adenovirus in the Treatment Of Neuropathology

Gene therapy vectors such as adenoviral, adeno-associated virus and lentiviral vectors are effective exogenously administered agents for inducing in vivo production of a protein, and are designed to provide long lasting, steady state protein levels at a specific site in vivo.

To determine the effects of exogenous VEGF-C or VEGF-D on neural stem cells in vivo, viral gene therapy vectors were employed. For example, adenoviral expression vectors containing VEGF-C (AdVEGF-C) or nuclear targeted LacZ (Ad-LacZ) transgenes were constructed as described in Enholm et al., Circ. Res., 88:623-629 (2001); and Puumalainen et al., (supra). Briefly, for Ad-VEGF-C, a full-length human VEGF-C cDNA was cloned under the cytomegalovirus promoter in the pcDNA3 vector (Invitrogen). The SV40-derived polyadenylation signal of the vector was then exchanged for that of the human growth hormone gene, and the transcription unit was inserted into the pAdBgIII vector as a BamHI fragment. Replication-deficient recombinant E1-E3-deleted adenoviruses were produced in human embryonic kidney 293 cells and concentrated by ultracentrifugation as previously described (Puumalainen et al., Hum. Gene Ther., 9:1769-1774, 1998). Adenoviral preparations are analyzed to be free of helper viruses, lipopolysaccharide, and bacteriological contaminants (Laitinen et al., Hum. Gene Ther., 9:1481-1486, 1998).

Rodent models useful in the assessment of VEGF-C in neuropathology include but are not limited to: the N-methyl-4-phenyl-1,2,3,6-tetrahydropyridine (MPTP) mouse model of Parkinsons's disease (Crocker et al., J Neurosci. 23:4081-91, 2003), methamphetamine induced mouse model of PD (Brown et al., Genome Res. 12:868-84, 2002), 6-OHDA induced PD (Björklund et al., Proc. Natl. Acad. Sci. U.S.A. 99:2344-2349, 2002), a transgenic Tg2576 mouse model of Alzheimer's disease (Quinn et al., J Neuroimmunol. 137:32-41. 2003), and the PDAPP mouse model of AD (Hartman et al., J Neurosci. 22:10083-7. 2002). The role of VEGF-C in neural trauma is assessed using a rat transection model (e.g. transection of fourth thoracic vertebra as described in Krassioukov, et al. (Am. J. Physiol. 268:H2077-H2083, 1995) and a spinal cord compression model (Gorio et al., Proc Natl. Acad. Sci. U.S.A. 99:9450-5, 2002).

VEGF-C adenoviral vector (Ad-VEGF-C) or LacZ control (Laitinen et al, supra) adenoviruses are injected at varying concentrations (ranging from 5×10⁶ to 5×10⁹ plaque forming units (pfu) into susceptible mice. The adenoviral vectors are administered either i.v., i.p., sub-cutaneously, intra-cranially or locally at the site of nervous system trauma. Ad-VEGF-C is administered before the onset of Alzheimer's or Parkinson's Disease neurodegenerative-like symptoms.

For Parkinson's disease, treated and control animals are monitored for progression of disease as above and are sacrificed at varying times after disease onset (d3, d7, d10, d14 or day 21 post onset) for histological assessment of neural proliferation, VEGF-C expression and neural cell differentiation as described above. In another embodiment, the adenoviral vectors are administered at varying times during the course of disease, including day 0, day 1, day 3, day 7, day 14, day 21 post induction or at times after the onset of disease to investigate the administration of VEGF-C on the progression and amelioration of neuronal disease. It is further contemplated that the adenoviral vector is administered multiple times on any of the days after onset of disease symptoms, to maintain a constant level of VEGF-C protein at the site of neuropathology.

Alzheimer's disease models generally require a longer development time in animal models. Assessment of the administration of VEGF-C on the progression of AD is determined several weeks to several months after birth of the transgenic animals or induction of disease in an experimentally-induced model of disease. VEGF-C treatment is administered at varying timepoints before the onset of AD symptoms. VEGF-C treated animals are sacrificed when control animals begin to exhibit signs of disease, and brain sections assayed for the extent of neurodegeneration and plaque formation. It is also contemplated that VEGF-C treatment is not administered until the first clinical sign of AD, and is then administered over varying timepoints at predetermined dosages. It is contemplated that VEGF-C or VEGF-D is administered daily, weekly, biweekly, or at other intervals determined to be effective for slowing the progression of AD.

Improvement of the disease symptoms or delay of disease progression in any of the animal models after VEGF-C treatment indicates a therapeutic benefit for VEGF-C to inhibit or reverse neurodegenerative disease progression.

### EXAMPLE 12

### Administration of Ex Vivo VEGF-C- or VEGF-D-Treated Neural Stem Cells

Neural stem cells are treated ex vivo with VEGF-C product or VEGF-D to induce the cells to proliferate. These cells are then implanted into a subject in need of neuronal generation and proliferation.

The use of neural stem cells as graft material has been illustrated by the neural progenitor clone, C17.2 [See U.S. Patent Publication No. 2002/0045261; Snyder et al., Cell 68: 33-51 1992; Snyder et al., Nature 374: 367-370, 1995; Park, J Neurotrauma 16: 675-87, 1999; Aboody-Guterman et al., NeuroReport 8: 3801-08. 1997]. C17.2 is a mouse cell line from postnatal day 0 cerebellum immortalized by infection with a retroviral construct containing the avian myc gene. This line has been transduced to constitutively express the lacZ and neoR genes. C17.2 cells transplanted into germinal zones throughout the brain can migrate, cease dividing, and participate in the normal development of multiple regions at multiple stages (fetus to adult) along the murine neuraxis, differentiating into diverse neuronal and glial cell types as expected. This clone of neural stem cells has been shown to be an effective vehicle for gene transfer to the CNS [Snyder et al., Nature 374: 367-70, 1995; Lacorraza et al., Nature Med 4: 424-29, 1996].

In one example, neural stem cells are cultured in vitro with VEGF-C beads as described above with an optimal concentration of soluble VEGF-C effective to stimulate growth and proliferation of the neural stem cells. The concentration of VEGF-C is optimized using techniques commonly used in the art, such as proliferation rate of cells over a given time period, changes in morphology, or state of cellular differentiation. Once optimized, VEGF-C is cultured with neural stem cells in vitro for a this optimal time period, e.g. 48 hours as in bead experiments.

Neural stem cells cultured with VEGF-C are then implanted into nu·nu mice as described in U.S. Patent Publication No. 2002/0045261. Intracerebral injection of neural stem cells is carried out as follows: male 6-8 weeks old nu/nu nude mice are anesthetized using an effective dose of anesthetic. e.g. by intraperitoneal (i.p.) injection with 70 μl of a solution consisting of 2 parts bacteriostatic 0.9% NaCl (Abbott Labs, Abbott, Ill.), and 1 part each of 20 mg/ml xylazine (Rompun, Miles. Kans.) and 100 mg/ml ketamine (Ketalar™., Parke-Davis, N.J.). The animals are positioned in a stereotactic apparatus (Kopf, Tujunga, Calif.), and a midline skin incision is made, and a burr hole drilled 2 mm rostral and 2 mm right of bregma. Cells are injected over a period of at least 2 min to a depth of 2.5 mm from the dura using a Hamilton syringe. The needle is gradually retracted over 2 min, the burr hole closed with bone wax (Ethicon, Somerville, N.J.), and the wound washed with Betadine (Purdue Frederick, Norwalk, Conn.). For secondary injections the same procedure is repeated.

Animals are sacrificed over a time course, e.g. day 2, day 4, day 5, day 6, day 7, day 10, day 14 or day 21 to assess the migration of VEGF-C treated stem cells. Animals are given an overdose of anesthesia and subsequent intracardiac perfusion with PBS followed by 4% paraformaldehyde and 2 mM MgCl₂ (pH 7.4). Brains are removed and post-fixed overnight at 4° C. and then transferred to 30% sucrose in PBS and 2 mM MgCl₂ (pH 7.4) for 3-7 days to cryoprotect the sample. Brains are stored at −80° C. and then 10-15 micron coronal serial sections are cut using a cryostat (Leica CM 3000, Wetzlar, Germany). It is also contemplated that neural stem cells are transfected with a marker protein such as LacZ as is commonly done in the art. These cells are treated with VEGF-C in culture as above, or with irrelevant control protein, e.g. bovine serum albumin, injected into animals and are subsequently easily traceable in vivo based on β-gal staining due to the presence of the LacZ gene.

Brain sections are stained to determine the extent of proliferation, migration and differentiation of VEGF-C treated neural stem cells. An increase in in vivo numbers of neural stem cells in the VEGF-C treated population or an overall increase in neural derived cells as compared to control group and assessment of their migration to appropriate sites after proliferation indicates that VEGF-C is a potent stimulator of neuronal growth and provides a useful therapy for the treatment of patients in need of neuronal regeneration. A change in tissue distribution of the VEGF-C treated cells provides an indication as to migration and differentiation effects of VEGF-C on the cells.

Neural stem cell transplantation described above is used in animal models of Parkinson's disease, Alzheimer's disease, or other neurodegenerative diseases to assess the ability of the VEGF-C or VEGF-D treated neural stem cells to improve neuropathology in a chronic neurodegenerative disease.

For example, VEGF-C treated neural stem calls are transplanted into mice affected by the (MPTP) mouse model of Parkinsons's disease (Crocker et al. supra). Neural stem cells are administered at varying times during the course of disease, either before or after disease onset, including day 0, day 1, day 3, day 7, day 14, or day 21 post disease induction, to investigate the administration of VEGF-C treated neural stem cells on the progression and amelioration of neuronal disease. Animals are sacrificed over a time course, e.g. day 2, day 4, day 5, day 6, day 7, day 10, day 14 or day 21 after neural stem cell transplantation to assess the migration of VEGF-C treated stem cells and measure the degree of improvement in brain lesions compared to control treated mice. A decrease in brain lesion size or improvement in motor skills in PD animals receiving VEGF-C treated stem cells indicates that VEGF-C acts as a potent activator of neural stem cell proliferation is a useful therapeutic for ameliorating the effects of neurodegenerative disease.

### EXAMPLE 13

### VEGF-C or VEGF-D Therapy in Patients with Neurodegenerative Disease

A. Treatment of Patients with Exogenous VEGF-C or VEGF-D

Patients exhibiting symptoms of a neurodegenerative disease or who have endured neural trauma or injury are treated with VEGF-C or VEGF-D products to promote regeneration, differentiation and migration of neural stem cells or neuronal progenitor cells.

In patients exhibiting signs of neurodegenerative disease, VEGF-C or VEGF-D products, as described previously, are administered to affected patients directly into the brain, e.g. intracerebroventricularly or intraputaminal injection, or by use of a catheter and infusion pump (Olson, L. Exp. Neurol. 124:5-15 (1993). VEGF-C or VEGF-D is administered in a therapeutically effective amount predetermined to be non-toxic to patients. VEGF-C- or EGF-D may be administered in one single dose or in multiple doses, and multiple doses may be given either in one day or over a timecourse determined by the treating physician to be most efficacious.

It is also contemplated that the VEGF-C or VEGF-D product is administered into the cerebrospinal fluid (CSF) of patients with neurodegenerative disease or patients suffering from neural trauma or injury.

For patients suffering from neural trauma or injury, VEGF-C or VEGF-D may also be also administered systemically via intravenous or subcutaneous injection in a therapeutically effective amount of VEGF-C/D product, or may be administered locally at the site of neural injury or trauma. Dosing (i.e. concentration of therapeutic and administration regimen) are determined by the administering physician and may be tailored to the patient being treated.

B. Transplant of VEGF-C or VEGF-D Treated Stem Cells to Patients with Neurodegenerative Disease.

Cells having the characteristics of multipotent neural stem cells, neuronal progenitors, or glial progenitors of the CNS (identified by in vitro assays) are treated with VEGF-C or VEGF-D product or infected with viral vectors expressing VEGF-C or VEGF-D product (e.g. adenoviral, adeno-associated, or lentiviral vectors), and are administered to a mammal exhibiting a neurological disorder to measure the therapeutic efficacy of these cells.

The cells are preferably isolated from a mammal having similar MHC genotypes. In one method, embryonic stem cell lines are isolated and cultured to induce differentiation toward a neuronal cell fate. This is done using neuronal growth factors as outlined above. Cells can be assessed for their state of differentiation based on cell surface staining for neuronal or glial cell lineage. These cells are subsequently cultured with VEGF-C and transferred into patients suffering from a neurodegenerative disease.

Isolation of neuronal stem cells is carried out as described in U.S. Pat. No. 5,196,315. In one instance, cerebral cortical tissue is obtained from a patient who may be undergoing treatment for their neuropathology or from removal of a neuronal tumor. Cortical tissue is dissected into gray and white matter, and the gray matter is immediately placed in minimal essential medium containing D-valine (MDV) (Gibco, Grand Island. N.Y.) and 15% dialyzed fetal bovine serum (dFBS) (Gibco), prepared by dialysis in tubing with a 12.000 to 14.000-dalton cut-off. Tissue is then finely minced and pushed through a 150-μm mesh wire screen. This cell suspension is distributed among 35-mm culture wells at a density of approximately 1×10⁴ cells per square centimeter and placed in a. 7% CO₂ humidified incubator at 37° C. The cell lines are maintained in MDV containing 15% dFBS and passaged by trypsinization [0.05% (w/v) in Hanks' balanced salt solution (Gibco)]. Cells are treated in vitro with varying concentration of VEGF-C or VEGF-D or transfected with viral vectors expressing either VEGF-C or VEGF-D.

The cultured cells are injected into the spinal cord or brain or other site of neural trauma or degeneration. The cells are injected at a range of concentrations to determine the optimal concentration into the desired site, and are microinjected into the brain and neurons of a subject animal.

Alternatively, the cells are introduced in a plasma clot, collagen gel or other slow release system to prevent rapid dispersal of cells from the site of injection. The slow release system is subsequently transplanted into the subject at or near the site of neuropathology. For example, to treat a patient suffering from Parkinson's disease, sufficient cells for grafting (assuming a 20% viability) are isolated from fetal/embryonic or adult brain tissue from surgical specimen or post-mortem donation which is homogenized and labeled with an neural stem cell marker. The cells are then sorted using fluorescence activated cell sorting (FACS). The cells which are neural marker positive are collected and further grown in tissue culture and treated. The cells are then transplanted into the striatum or the substantia nigra of a Parkinson's patient. The transplant is monitored for viability and differentiation of the cells.

It is contemplated that VEGF-C or VEGF-D treatment is used in conjunction with therapies commonly used to treat neurodegenerative diseases. For example, in one regimen for the treatment of a patient with Parkinson's disease, patients receive a neurotherapeutic agent such as pramipexole or levodopa, at a dose of 0.5 mg 3 times per day in conjunction with VEGF-C treatment, or after administration of VEGF-C cultured neural stem cells. Alternatively, patients receive carbidopa/levodopa, 25/100 mg 3 times per day either before, concurrent with, or after VEGF-C treatment or after transplantation of VEGF-C treated neural stem cells. If patients exhibit continued disability, the dosage is escalated during the first 10 weeks. It is well known in the art that treatment regimens are often modified and optimized by the treating physician and are patient specific. As such, the dosage of any of the chemotherapeutic agents may be further modified and given in any combination that proves effective at ameliorating the effects of the neurodegenerative disease. For example, if coenzyme Q10 is used as the therapeutic, it may be given at a dose range 300, or 600, or 1200 mg/day in conjunction with VEGF-C product

These techniques and methods are used in the treatment of neurological degenerative diseases such as Alzheimer's disease or Parkinson's disease, or in the treatment of a traumatic injury in which neuronal cells are damaged, such as during strokes. The effect of treatment on the neurological status of the subject patient is monitored. For instance, proliferation of neuronal stem cells in vivo can be detected by MRI. Desired therapeutic effects in the subject include improved motor-neuron function and decreased neuronal scarring or neuronal lesions in a subject affected by neuropathology.

Any of the above examples are performed using VEGF-D products in place of VEGF-C products. It is contemplated that VEGF-D produces similar neural cell growth stimulatory activity as VEGF-C and is used in much the same way as VEGF-C in administering to individuals suffering from a neuropathology or to stimulate neural cell growth in vitro for transplantation to patients exhibiting symptoms of neuropathology. Additionally, VEGF-D expressing viral vectors are used as gene therapy as described above for VEGF-C.

Practicing the Examples using small organic or inorganic molecules identified by screening peptide libraries or chemical compound libraries, in place of the neuropilin or VEGF-C polypeptides is particularly contemplated. Small molecules and chemical compounds identified as modulators of neuropilin/VEGF-C, VEGFR-3VEGF-C and/or neuropilin/VEGFR-3 interactions will be useful as therapeutic compositions to treat situations requiring neuronal cell growth and regeneration, and in the manufacture of a medicament for the treatment of diseases characterized by aberrant growth, migration, or proliferation of neuronal cells mediated by VEGF-C activity.

The foregoing describes and exemplifies the invention but is not intended to limit the invention defined by the claims which follow:

What is claimed is:

1. A method of promoting recruitment, proliferation, differentiation, migration or survival of neuronal cells or neuronal precursor cells in a mammalian subject comprising administering to the subject a composition comprising a vascular endothelial growth factor C (VEGF-C) product or a vascular endothelial growth factor D (VEGF-D) product.

2. The method of claim 1 further comprising a step, prior to the administrating step, of identifying a mammalian subject in need of neuronal cell or neuronal precursor cell recruitment, proliferation, or differentiation.

3. The method of claim 2 wherein the VEGF-C product comprises a purified mammalian prepro-VEGF-C polypeptide, VEGF-C ΔNΔC, VEGF-C ΔC₁₅₆, VEGF-C ΔNΔC C156S, a chimeric heparin-binding VEGF-C, or a fragment of the prepro-VEGF-C polypeptide that binds a VEGF-C receptor, wherein the VEGF-C receptor is selected from the group consisting of VEGFR-3, VEGFR-2, neuropilin-1 and neuropilin-2.

4. The method of claim 3 wherein the subject and the prepro-VEGF-C polypeptide are human.

5. The method of claim 4 wherein the VEGF-C product comprises a fragment of human prepro-VEGF-C that contains amino acids 32-227 of SEQ ID NO: 24.

6. The method of claim 2 wherein the VEGF-C product comprises a polynucleotide selected from the group consisting of:
(a) a polynucleotide comprising a nucleotide sequence that encodes the human VEGF-C amino acid sequence of SEQ ID NO: 24:
(b) a polynucleotide comprising a nucleotide sequence at least 90% identical to the nucleotide sequence of SEQ ID NO: 23 and encodes a polypeptide that binds VEGFR-3;
(c) a polynucleotide comprising a nucleotide sequence that encodes a polypeptide comprising an amino acid sequence at least 90% identical to SEQ ID NO: 24, wherein the polypeptide binds VEGFR-3;
(d) a polynucleotide that hybridizes to the complement of SEQ ID NO: 23 under the following stringent conditions and encodes a polypeptide that binds VEGFR-3: 2×SSC/0.1% SDS twice at RT, 1×SSC/0.1% SDS 15 min at 55° C., 0.1×SSC/0.1% SDS 15 min at 55° C.
(e) fragments of (a)-(d) that encoded a polypeptide that binds VEGFR-3.

7. The method of claim 2, wherein the VEGF-C product comprises a polynucleotide that encodes a VEGF-C polypeptide set forth in SEQ ID NO: 24 or fragment thereof that binds VEGFR-3.

8. The method of claim 6 or 7, wherein the VEGF-C product comprises a viral vector containing the polynucleotide.

9. The method of claim 8, wherein the vector comprises a replication-deficient adenovirus, adeno-associated virus, or lentivirus.

10. A method according to claim 2 wherein the composition further comprises a pharmaceutically acceptable carrier.

11. A method of stimulating neural stem cell proliferation or differentiation, comprising,
obtaining a biological sample from a mammalian subject, wherein said sample comprises neural stem cells, and
contacting the neural stem cells with a composition comprising a vascular endothelial growth factor C (VEGF-C) product or a vascular endothelial growth factor D (VEGF-D) product.

12. A method according to claim 11, wherein the contacting comprises culturing the stem cells in a culture containing VEGF-C product or VEGF-D product.

13. A method according to claim 11, further comprising a step of purifying and isolating the neural stem cells from the sample before the contacting step.

14. A method according to any one of claims 11-13, further comprising a step of purifying and isolating neural stem cells or neural cells after the contacting step.

15. Purified and isolated neural cells cultured according to claim 14.

16. The method according to claim 13, further comprising a step of administering the neural stem cells to the mammalian subject after the contacting step.

17. The method according to claim 13, further comprising a step of transplanting the neural stem cells into a different mammalian subject after the contacting step.

18. The method of claim 16 or 17, wherein the cells are seeded into a tissue, organ, or artificial matrix ex vivo, and said tissue, organ, or artificial matrix is attached, implanted, or transplanted into the mammalian subject

19. A method of inducing neural stem cell proliferation in vitro comprising contacting the neural stem cell with a composition comprising a VEGF-C product or a VEGF-D product, wherein the neural stem cell is selected from the group consisting of C17.2, purified neural stem cells, HSN-1 cells, fetal pig cells, neural crest cells, bone marrow derived neural stem cells, hNT cells and a human neuronal progenitor cell line.

20. A method according to claim 19, further comprising a step of administering the stem cells to a mammalian subject after the contacting step.

21. The method of claim 19, wherein the cells are seeded into a tissue, organ, or artificial matrix ex vivo, and said tissue, organ, or artificial matrix is attached, implanted, or transplanted into a mammalian subject.

22. The method of claim 16, 17, or 20 wherein the mammalian subject is human.

23. The method of claim 1 or 16 wherein the VEGF-C or VEGF-D product is administered in conjunction with a neural growth factor.

24. The method of claim 23 wherein the neural growth factor is selected from the group consisting of interferon gamma, nerve growth factor, epidermal growth factor (EGF), basic fibroblast growth factor (bFGF), neurogenin, brain derived neurotrophic factor (BDNF), thyroid hormone, bone morphogenic proteins (BMPs), leukemia inhibitory factor (LIF), sonic hedgehog, glial cell line-derived neurotrophic factor (GDNFs), vascular endothelial growth factor (VEGF), interleukins, interferons, stem cell factor (SCF), activins, inhibins, chemokines, retinoic acid and ciliary neurotrophic factor (CNTF).

25. A method according to claim 1 or 16, wherein the subject has a disease or condition characterized by aberrant growth of neuronal cells, neuronal scarring, or neural degeneration.

26. A method according to claim 25, wherein the neural degeneration is caused by a neurodegenerative disorder selected from the group consisting of is Alzheimer's disease, Parkinson's disease, Huntington's disease, motor neuron disease, Amyotrophic Lateral Sclerosis (ALS), dementia and cerebral palsy.

27. A method according to claim 1 or 16, wherein the disease or condition is selected from the group consisting of neural trauma or neural injury.

28. The method of claim 27, wherein the neural trauma is selected from the group consisting of stroke-related injury, spinal cord injury, post-operative injury and brain ischemia.

29. The method of claim 1 or 13 wherein the VEGF-C product is administered in conjunction with a neurotherapeutic agent.

30. The method of claim 29 wherein the neurotherapeutic agent is selected form the group consisting of tacrine (Cognex), donepezil (Aricept), rivastigmine (Exelon), galantamine (Reminyl), cholinesterase inhibitors and anti-inflammatory drugs.

31. The method of claim 29 wherein the neurotherapeutic agent is selected form the group consisting of anti-cholinergics, dopamine agonists, catechol-O-methyl-transterases (COMTs), amantadine (Symmetrel), Sinemet®, Selegiline, carbidopa, ropinirole (Requip). coenzyme Q10, Pramipexole (Mirapex) and levodopa (L-dopa).

32. A method of inhibiting growth and progression and of neuroblastoma and neural tumors comprising administering to a subject having a neuroblastoma or neuronal tumor a composition comprising a VEGF-C inhibitor.

33. The method of claim 32 wherein the VEGF-C inhibitor is selected from the group consisting of a polypeptide comprising an extracellular fragment of VEGFR-2 that binds to VEGF-C, VEGF-C neutralizing antibodies, VEGF-C antisense molecules, siRNA, and small molecule inhibitors.

34. The method of claim 32 wherein the VEGF-C inhibitor is selected from the group consisting of a polypeptide comprising an extracellular fragment of VEGFR-3 that binds to VEGF-C, an extracellular fragment of NRP-1 that binds to VEGF-C, and an extracellular fragment of NRP-2 that binds to VEGF-C.

35. A composition comprising a VEGF-C product and a neural growth factor in a pharmaceutically acceptable diluent or carrier.

36. A composition comprising a VEGF-C product and a neurotherapeutic agent in a pharmaceutically acceptable diluent or carrier.


SEQUENCE LISTING (38 sequences)

SEQ ID NO: 1 - DNA, 2772 bp, Homo sapiens
  CDS (1)..(2772)
atggagagggggctgccgctcctctgcgccgtgctcgccctcgtcctcgccccggccggcgcttttcgcaacgatgaatgtggcgatactataaaaattgaaagccccgggtaccttacatctcctggttatcctcattcttatcacccaagtgaaaaatgcgaatggctgattcaggctccggacccataccagagaattatgatcaacttcaaccctcacttcgatttggaggacagagactgcaagtatgactacgtggaagtcttcgatggagaaaatgaaaatggacattttaggggaaagttctgtggaaagatagcccctcctcctgttgtgtcttcagggccatttctttttatcaaatttgtctctgactacgaaacacatggtgcaggattttccatacgttatgaaattttcaagagaggtcctgaatgttcccagaactacacaacacctagtggagtgataaagtcccccggattccctgaaaaatatcccaacagccttgaatgcacttatattgtctttgcgccaaagatgtcagagattatcctggaatttgaaagctttgacctggagcctgactcaaatcctccaggggggatgttctgtcgctacgaccggctagaaatctgggatggattccctgatgttggccctcacattgggcgttactgtggacagaaaacaccaggtcgaatccgatcctcatcgggcattctctccatggttttttacaccgacagcgcgatagcaaaagaaggtttctcagcaaactacagtgtcttgcagagcagtgtctcagaagatttcaaatgtatggaagctctgggcatggaatcaggagaaattcattctgaccagatcacagcttcttcccagtatagcaccaactggtctgcagagcgctcccgcctgaactaccctgagaatgggtggactcccggagaggattcctaccgagagtggatacaggtagacttgggccttctgcgctttgtcacggctgtcgggacacagggcgccatttcaaaagaaaccaagaagaaatattatgtcaagacttacaagatcgacgttagctccaacggggaagactggatcaccataaaagaaggaaacaaacctgttctctttcagggaaacaccaaccccacagatgttgtggttgcagtattccccaaaccactgataactcgatttgtccgaatcaagcctgcaacttgggaaactggcatatctatgagatttgaagtatacggttgcaagataacagattatccttgctctggaatgttgggtatggtgtctggacttatttctgactcccagatcacatcatccaaccaaggagacagaaactggatgcctgaaaacatccgcctggtaaccagtcgctctggctgggcacttccacccgcacctcattcctacatcaatgagtggctccaaatagacctgggggaggagaagatcgtgaggggcatcatcattcagggtgggaagcaccgagagaacaaggtgttcatgaggaagttcaagatcgggtacagcaacaacggctcggactggaagatgatcatggatgacagcaaacgcaaggcgaagtcttttgagggcaacaacaactatgatacacctgagctgcggacttttccagctctctccacgcgattcatcaggatctaccccgagagagccactcatggcggactggggctcagaatggagctgctgggctgtgaagtggaagcccctacagctggaccgaccactcccaacgggaacttggtggatgaatgtgatgacgaccaggccaactgccacagtggaacaggtgatgacttccagctcacaggtggcaccactgtgctggccacagaaaagcccacggtcatagacagcaccatacaatcagagtttccaacatatggttttaactgtgaatttggctggggctctcacaagaccttctgccactgggaacatgacaatcacgtgcagctcaagtggagtgtgttgaccagcaagacgggacccattcaggatcacacaggagatggcaacttcatctattcccaagctgacgaaaatcagaagggcaaagtggctcgcctggtgagccctgtggtttattcccagaactctgcccactgcatgaccttctggtatcacatgtctgggtcccacgtcggcacactcagggtcaaactgcgctaccagaagccagaggagtacgatcagctggtctggatggccattggacaccaaggtgaccactggaaggaagggcgtgtcttgctccacaagtctctgaaactttatcaggtgattttcgagggcgaaatcggaaaaggaaaccttggtgggattgctgtggatgacattagtattaataaccacatttcacaagaagattgtgcaaaaccagcagacctggataaaaagaacccagaaattaaaattgatgaaacagggagcacgccaggatacgaaggtgaaggagaaggtgacaagaacatctccaggaagccaggcaatgtgttgaagaccttagaacccatcctcatcaccatcatagccatgagcgccctgggggtcctcctgggggctgtctgtggggtcgtgctgtactgtgcctgttggcataatgggatgtcagaaagaaacttgtctgccctggagaactataactttgaacttgtggatggtgtgaagttgaaaaaagacaaactgaatacacagagtacttattcggaggcatga

SEQ ID NO: 2 - protein, 923 aa, Homo sapiens
MERGLPLLCAVLALVLAPAGAFRNDECGDTIKIESPGYLTSPGYPHSYHPSEKCEWLIQAPDPYQRIMINFNPHFDLEDRDCKYDYVEVFDGENENGHFRGKFCGKIAPPPVVSSGPFLFIKFVSDYETHGAGFSIRYEIFKRGPECSQNYTTPSGVIKSPGFPEKYPNSLECTYIVFAPKMSEIILEFESFDLEPDSNPPGGMFCRYDRLEIWDGFPDVGPHIGRYCGQKTPGRIRSSSGILSMVFYTDSAIAKEGFSANYSVLQSSVSEDFKCMEALGMESGEIHSDQITASSQYSTNWSAERSRLNYPENGWTPGEDSYREWIQVDLGLLRFVTAVGTQGAISKETKKKYYVKTYKIDVSSNGEDWITIKEGNKPVLFQGNTNPTDVVVAVFPKPLITRFVRIKPATWETGISMRFEVYGCKITDYPCSGMLGMVSGLISDSQITSSNQGDRNWMPENIRLVTSRSGWALPPAPHSYINEWLQIDLGEEKIVRGIIIQGGKHRENKVFMRKFKIGYSNNGSDWKMIMDDSKRKAKSFEGNNNYDTPELRTFPALSTRFIRIYPERATHGGLGLRMELLGCEVEAPTAGPTTPNGNLVDECDDDQANCHSGTGDDFQLTGGTTVLATEKPTVIDSTIQSEFPTYGFNCEFGWGSHKTFCHWEHDNHVQLKWSVLTSKTGPIQDHTGDGNFIYSQADENQKGKVARLVSPVVYSQNSAHCMTFWYHMSGSHVGTLRVKLRYQKPEEYDQLVWMAIGHQGDHWKEGRVLLHKSLKLYQVIFEGEIGKGNLGGIAVDDISINNHISQEDCAKPADLDKKNPEIKIDETGSTPGYEGEGEGDKNISRKPGNVLKTLEPILITIIAMSALGVLLGAVCGVVLYCACWHNGMSERNLSALENYNFELVDGVKLKKDKLNTQSTYSEA

SEQ ID NO: 3 - DNA, 2781 bp, Homo sapiens
  CDS (1)..(2781)
atggatatgtttcctctcacctgggttttcttagccctctacttttcaagacaccaagtgagaggccaaccagacccaccgtgcggaggtcgtttgaattccaaagatgctggctatatcacctctcccggttacccccaggactacccctcccaccagaactgcgagtggattgtttacgcccccgaacccaaccagaagattgtcctcaacttcaaccctcactttgaaatcgagaagcacgactgcaagtatgactttatcgagattcgggatggggacagtgaatccgcagacctcctgggcaaacactgtgggaacatcgccccgcccaccatcatctcctcgggctccatgctctacatcaagttcacctccgactacgcccggcagggggcaggcttctctctgcgctacgagatcttcaagacaggctctgaagattgctcaaaaaacttcacaagccccaacgggaccatcgaatctcctgggtttcctgagaagtatccacacaacttggactgcacctttaccatcctggccaaacccaagatggagatcatcctgcagttcctgatctttgacctggagcatgaccctttgcaggtgggagagggggactgcaagtacgattggctggacatctgggatggcattccacatgttggccccctgattggcaagtactgtgggaccaaaacaccctctgaacttcgttcatcgacggggatcctctccctgacctttcacacggacatggcggtggccaaggatggcttctctgcgcgttactacctggtccaccaagagccactagagaactttcagtgcaatgttcctctgggcatggagtctggccggattgctaatgaacagatcagtgcctcatctacctactctgatgggaggtggacccctcaacaaagccggctccatggtgatgacaatggctggacccccaacttggattccaacaaggagtatctccaggtggacctgcgctttttaaccatgctcacggccatcgcaacacagggagcgatttccagggaaacacagaatggctactacgtcaaatcctacaagctggaagtcagcactaatggagaggactggatggtgtaccggcatggcaaaaaccacaaggtatttcaagccaacaacgatgcaactgaggtggttctgaacaagctccacgctccactgctgacaaggtttgttagaatccgccctcagacctggcactcaggtatcgccctccggctggagctcttcggctgccgggtcacagatgctccctgctccaacatgctggggatgctctcaggcctcattgcagactcccagatctccgcctcttccacccaggaatacctctggagccccagtgcagcccgcctggtcagcagccgctcgggctggttccctcgaatccctcaggcccagcccggtgaggagtggcttcaggtagatctgggaacacccaagacagtgaaaggtgtcatcatccagggagcccgcggaggagacagtatcactgctgtggaagccagagcatttgtgcgcaagttcaaagtctcctacagcctaaacggcaaggactgggaatacattcaggaccccaggacccagcagccaaagctgttcgaagggaacatgcactatgacacccctgacatccgaaggtttgaccccattccggcacagtatgtgcgggtatacccggagaggtggtcgccggcggggattgggatgcggctggaggtgctgggctgtgactggacagactccaagcccacggtaaaaacgctgggacccactgtgaagagcgaagagacaaccaccccctaccccaccgaagaggaggccacagagtgtggggagaactgcagctttgaggatgacaaagatttgcagctcccttcgggattcaattgcaacttcgatttcctcgaggagccctgtggttggatgtatgaccatgccaagtggctccggaccacctgggccagcagctccagcccaaacgaccggacgtttccagatgacaggaatttcttgcggctgcagagtgacagccagagagagggccagtatgcccggctcatcagcccccctgtccacctgccccgaagcccggtgtgcatggagttccagtaccaggccacgggcggccgcggggtggcgctgcaggtggtgcgggaagccagccaggagagcaagttgctgtgggtcatccgtgaggaccagggcggcgagtggaagcacgggcggatcatcctgcccagctacgacatggagtaccagattgtgttcgagggagtgatagggaaaggacgttccggagagattgccattgatgacattcggataagcactgatgtcccactggagaactgcatggaacccatctcggcttttgcagtggacatcccagaaatacatgagagagaaggatatgaagatgaaattgatgatgaatacgaggtggactggagcaattcttcttctgcaacctcagggtctggcgccccctcgaccgacaaagaaaagagctggctgtacaccctggatcccatcctcatcaccatcatcgccatgagctcactgggcgtcctcctgggggccacctgtgcaggcctcctgctctactgcacctgttcctactcgggcctgagctcccgaagctgcaccacactggagaactacaacttcgagctctacgatggccttaagcacaaggtcaagatgaaccaccaaaagtgctgctccgaggcatga

SEQ ID NO: 4 - protein, 926 aa, Homo sapiens
MDMFPLTWVFLALYFSRHQVRGQPDPPCGGRLNSKDAGYITSPGYPQDYPSHQNCEWIVYAPEPNQKIVLNFNPHFEIEKHDCKYDFIEIRDGDSESADLLGKHCGNIAPPTIISSGSMLYIKFTSDYARQGAGFSLRYEIFKTGSEDCSKNFTSPNGTIESPGFPEKYPHNLDCTFTILAKPKMEIILQFLIFDLEHDPLQVGEGDCKYDWLDIWDGIPHVGPLIGKYCGTKTPSELRSSTGILSLTFHTDMAVAKDGFSARYYLVHQEPLENFQCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFGCRVTDAPCSNMLGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKLFEGNMHYDTPDIRRFDPIPAQYVRVYPERWSPAGIGMRLEVLGCDWTDSKPTVKTLGPTVKSEETTTPYPTEEEATECGENCSFEDDKDLQLPSGFNCNFDFLEEPCGWMYDHAKWLRTTWASSSSPNDRTFPDDRNFLRLQSDSQREGQYARLISPPVHLPRSPVCMEFQYQATGGRGVALQVVREASQESKLLWVIREDQGGEWKHGRIILPSYDMEYQIVFEGVIGKGRSGEIAIDDIRISTDVPLENCMEPISAFAVDIPEIHEREGYEDEIDDEYEVDWSNSSSATSGSGAPSTDKEKSWLYTLDPILITIIAMSSLGVLLGATCAGLLLYCTCSYSGLSSRSCTTLENYNFELYDGLKHKVKMNHQKCCSEA

SEQ ID NO: 5 - DNA, 3652 bp, Mus musculus
  CDS (348)..(3119)
  misc_feature (348)..(410): Signal Peptide
tttttttttttttttttttttttttttttttttttcctccttcttcttcttcctgagacatggcccgggcagtggctcctggaagaggaacaagtgtgggaaaagggagaggaaatcggagctaaatgacaggatgcaggcgacttgagacacaaaaagagaagcgcttctcgcgaattcaggcattgcctcgccgctagccttccccgccaagacccgctgaggattttatggttcttaggcggacttaagagcgtttcggattgttaagattatcgtttgctggtttttcgtccgcgcaatcgtgttctcctgcggctgcctggggactggcttggcgaaggaggatggagagggggctgccgttgctgtgcgccacgctcgcccttgccctcgccctggcgggcgctttccgcagcgacaaatgtggcgggaccataaaaatcgaaaacccagggtacctcacatctcccggttaccctcattcttaccatccaagtgagaagtgtgaatggctaatccaagctccggaaccctaccagagaatcataatcaacttcaacccacatttcgatttggaggacagagactgcaagtatgactacgtggaagtaattgatggggagaatgaaggcggccgcctgtgggggaagttctgtgggaagattgcaccttctcctgtggtgtcttcagggccctttctcttcatcaaatttgtctctgactatgagacacatggggcagggttttccatccgctatgaaatcttcaagagagggcccgaatgttctcagaactatacagcacctactggagtgataaagtcccctgggttccctgaaaaataccccaactgcttggagtgcacctacatcatctttgcaccaaagatgtctgagataatcctggagtttgaaagttttgacctggagcaagactcgaatcctcccggaggaatgttctgtcgctatgaccggctggagatctgggatggattccctgaagttggccctcacattgggcgttattgtgggcagaaaactcctggccggatccgctcctcttcaggcgttctatccatggtcttttacactgacagcgcaatagcaaaagaaggtttctcagccaactacagtgtgctacagagcagcatctctgaagattttaagtgtatggaggctctgggcatggaatctggagagatccattctgatcagatcactgcatcttcacagtatggtaccaactggtctgtagagcgctcccgcctgaactaccctgaaaatgggtggactccaggagaagactcctacaaggagtggatccaggtggacttgggcctcctgcgattcgttactgctgtagggacacagggtgccatttccaaggaaaccaagaagaaatattatgtcaagacttacagagtagacatcagctccaacggagaggactggatctccctgaaagagggaaataaagccattatctttcagggaaacaccaaccccacagatgttgtcttaggagttttctccaaaccactgataactcgatttgtccgaatcaaacctgtatcctgggaaactggtatatctatgagatttgaagtttatggctgcaagataacagattatccttgctctggaatgttgggcatggtgtctggacttatttcagactcccagattacagcatccaatcaagccgacaggaattggatgccagaaaacatccgtctggtgaccagtcgtaccggctgggcactgccaccctcaccccacccatacaccaatgaatggctccaagtggacctgggagatgagaagatagtaagaggtgtcatcattcagggtgggaagcaccgagaaaacaaggtgttcatgaggaagttcaagatcgcctatagtaacaatggctctgactggaaaactatcatggatgacagcaagcgcaaggctaagtcgttcgaaggcaacaacaactatgacacacctgagcttcggacgttttcacctctctccacaaggttcatcaggatctaccctgagagagccacacacagtgggcttgggctgaggatggagctactgggctgtgaagtggaagcacctacagctggaccaaccacacccaatgggaacccagtgcatgagtgtgacgacgaccaggccaactgccacagtggcacaggtgatgacttccagctcacaggaggcaccactgtcctggccacagagaagccaaccattatagacagcaccatccaatcagagttcccgacatacggttttaactgcgagtttggctggggctctcacaagacattctgccactgggagcatgacagccatgcacagctcaggtggagtgtgctgaccagcaagacagggccgattcaggaccatacaggagatggcaacttcatctattcccaagctgatgaaaatcagaaaggcaaagtagcccgcctggtgagccctgtggtctattcccagagctctgcccactgtatgaccttctggtatcacatgtccggctctcatgtgggtacactgagggtcaaactacgctaccagaagccagaggaatatgatcaactggtctggatggtggttgggcaccaaggagaccactggaaagaaggacgtgtcttgctgcacaaatctctgaaactatatcaggttatttttgaaggtgaaatcggaaaaggaaaccttggtggaattgctgtggatgatatcagtattaacaaccatatttctcaggaagactgtgcaaaaccaacagacctagataaaaagaacacagaaattaaaattgatgaaacagggagcactccaggatatgaaggagaaggggaaggtgacaagaacatctccaggaagccaggcaatgtgcttaagaccctggatcccatcctgatcaccatcatagccatgagtgccctgggagtactcctgggtgcagtctgtggagttgtgctgtactgtgcctgttggcacaatgggatgtcagaaaggaacctatctgccctggagaactataactttgaacttgtggatggtgtaaagttgaaaaaagataaactgaacccacagagtaattactcagaggcgtgaaggcacggagctggagggaacaagggaggagcacggcaggagaacaggtggaggcatggggactctgttactctgctttcactgtaagctgggaagggcggggactctgttactccgctttcactgtaagctcggaagggcatccacgatgccatgccaggcttttctcaggagcttcaatgagcgtcacctacagacacaagcaggtgactgcggtaacaacaggaatcatgtacaagcctgctttcttctcttggtttcatttgggtaatcagaagccatttgagaccaagtgtgactgacttcatggttcatcctactagcccccttttttcctctctttctccttaccctgtggtggattcttctcggaaactgcaaaatccaagatgctggcactaggcgttattcagtgggcccttttgatggacatgtgacctgtagcccagtgcccagagcatattatcataaccacatttcaggggacgccaacgtccatccacctttgcatcgctacctgcagcgagcacagg

SEQ ID NO: 6 - protein, 923 aa, Mus musculus
  misc_feature (348)..(410): Signal Peptide
MERGLPLLCATLALALALAGAFRSDKCGGTIKIENPGYLTSPGYPHSYHPSEKCEWLIQAPEPYQRIIINFNPHFDLEDRDCKYDYVEVIDGENEGGRLWGKFCGKIAPSPVVSSGPFLFIKFVSDYETHGAGFSIRYEIFKRGPECSQNYTAPTGVIKSPGFPEKYPNCLECTYIIFAPKMSEIILEFESFDLEQDSNPPGGMFCRYDRLEIWDGFPEVGPHIGRYCGQKTPGRIRSSSGVLSMVFYTDSAIAKEGFSANYSVLQSSISEDFKCMEALGMESGEIHSDQITASSQYGTNWSVERSRLNYPENGWTPGEDSYKEWIQVDLGLLRFVTAVGTQGAISKETKKKYYVKTYRVDISSNGEDWISLKEGNKAIIFQGNTNPTDVVLGVFSKPLITRFVRIKPVSWETGISMRFEVYGCKITDYPCSGMLGMVSGLISDSQITASNQADRNWMPENIRLVTSRTGWALPPSPHPYTNEWLQVDLGDEKIVRGVIIQGGKHRENKVFMRKFKIAYSNNGSDWKTIMDDSKRKAKSFEGNNNYDTPELRTFSPLSTRFIRIYPERATHSGLGLRMELLGCEVEAPTAGPTTPNGNPVHECDDDQANCHSGTGDDFQLTGGTTVLATEKPTIIDSTIQSEFPTYGFNCEFGWGSHKTFCHWEHDSHAQLRWSVLTSKTGPIQDHTGDGNFIYSQADENQKGKVARLVSPVVYSQSSAHCMTFWYHMSGSHVGTLRVKLRYQKPEEYDQLVWMVVGHQGDHWKEGRVLLHKSLKLYQVIFEGEIGKGNLGGIAVDDISINNHISQEDCAKPTDLDKKNTEIKIDETGSTPGYEGEGEGDKNISRKPGNVLKTLDPILITIIAMSALGVLLGAVCGVVLYCACWHNGMSERNLSALENYNFELVDGVKLKKDKLNPQSNYSEA

SEQ ID NO: 7 - DNA, 4769 bp, Mus musculus
  CDS (567)..(3347)
aaactggagctccaccgcggtggcggccgcccgggcaggtctagaattcagcggccgctgaattctatccagcggtcggtgcctctgcccgcgtgtgtgtcccgggtgccgggggacctgtgtcagttagcgcttctgagatcacacagctgcctaggggccgtgtgatgcccagggcaattcttggctttgatttttattattattactattattttgcgttcagctttcgggaaaccctcgtgatgttgtaggataaaggaaatgacactttgaggaactggagagaacatacacgcgtttgggtttgaagaggaaaccggtctccgcttccttagcttgctccctctttgctgatttcaagagctatctcctatgaggtggagatattccagcaagaataaaggtgaagacagactgactgccaggacccaggaggaaaacgttgatcgttagagacctttgcagaagacaccaccaggaggaaaattagagaggaaaaacacaaagacataattataggagatcccacaaacctagcccgggagagagcctctctgtcaaaaatggatatgtttcctcttacctgggttttcttagctctgtacttttcaggacacgaagtgagaagccagcaagatccaccctgcggaggtcggccgaattccaaagatgctggctacatcacttccccaggctacccccaggactatccctcccaccagaactgtgagtggattgtctacgcccccgaacccaaccagaagattgttctcaacttcaaccctcactttgaaatcgagaaacacgactgcaagtatgacttcattgagattcgggatggggacagtgagtcagctgacctcctgggcaagcactgtgggaacatcgccccgcccaccatcatctcctcaggctccgtgttatacatcaagttcacctcagactacgcccggcagggggcaggtttctctctacgctatgagatcttcaaaacaggctctgaagattgttccaagaactttacaagccccaatgggaccattgaatctccagggtttccagagaagtatccacacaatctggactgtaccttcaccatcctggccaaacccaggatggagatcatcctacagttcctgacctttgacctggagcatgaccctctacaagtgggggaaggagactgtaaatatgactggctggacatctgggatggcattccacatgttggacctctgattggcaagtactgtgggacgaaaacaccctccaaactccgctcgtccacggggatcctctccttgacctttcacacggacatggcagtggccaaggatggcttctccgcacgttactatttgatccaccaggagccacctgagaattttcagtgcaatgtccctttgggaatggagtctggccggattgctaatgaacagatcagtgcctcctccaccttctctgatgggaggtggactcctcaacagagccggctccatggtgatgacaatggctggacacccaatttggattccaacaaggagtatctccaggtggacctgcgcttcctaaccatgctcacagccattgcaacacagggagccatttccagggaaacccagaaaggctactacgtcaaatcgtacaagctggaagtcagcacaaatggtgaagattggatggtctaccggcatggcaaaaaccacaagatattccaagcgaacaatgatgcgaccgaggtggtgctaaacaagctccacatgccactgctgactcggttcatcaggatccgcccgcagacgtggcatttgggcattgcccttcgcctggagctctttggctgccgggtcacagatgcaccctgctccaacatgctggggatgctctcgggcctcattgctgatacccagatctctgcctcctccacccgagagtacctctggagccccagtgctgcccgcctggttagtagccgctctggctggtttcctcggaaccctcaagcccagccaggtgaagaatggcttcaggtagacctggggacacccaagacagtgaaaggggtcatcatccagggagcccgaggaggagacagcatcactgccgtggaagccagggcgtttgtacgcaagttcaaagtctcctacagcctaaatggcaaggactgggaatatatccaggaccccaggactcagcagacaaagctgtttgaagggaacatgcactatgacacccctgacatccgaaggttcgatcctgttccagcgcagtatgtgcgggtgtacccagagaggtggtcgccagcaggcatcgggatgaggctggaggtgctgggctgtgactggacagactcaaagcccacagtggagacgctgggacccaccgtgaagagtgaagagactaccaccccatatcccatggatgaggatgccaccgagtgtggggaaaactgcagctttgaggatgacaaagatttgcaacttccttcaggattcaactgcaactttgattttccggaagagacctgtggttgggtgtacgaccatgccaagtggctccggagcacgtggatcagcagcgctaaccccaatgacagaacatttccagatgacaagaacttcttgaaactgcagagtgatggccgacgagagggccagtacgggcggctcatcagcccaccggtgcacctgccccgaagccctgtgtgcatggagttccagtaccaagccatgggcggccacggggtggcactgcaggtggttcgggaagccagccaggaaagcaaactcctttgggtcatccgtgaggaccagggcagcgagtggaagcacgggcgcattatcctgcccagctatgacatggagtatcagatcgtgttcgagggagtgatagggaagggacgatcgggagagatttccggcgatgacattcggataagcactgatgtcccactggagaactgcatggaacccatatcagcttttgcagtggacatcccagaaacccatgggggagagggctatgaagatgagattgatgatgaatatgaaggagattggagcaactcttcttcctctacctcaggggctggtgacccctcatctggcaaagaaaagagctggctgtacaccctagatcccattctgatcaccatcatcgccatgagctcgctgggggtcctgctgggggccacctgtgcgggcctcctcctttactgcacctgctcctattcgggtctgagttcgaggagctgcaccacactggagaactacaactttgagctctacgatggcctcaagcacaaggtcaagatcaatcatcagaagtgctgctcggaggcatgaccgattgtgtctggatcgcttctggcgtttcattccagtgagaggggctagcgaagattacagttttgttttgttttgttttgttttccctttggaaactgaatgccataatctggatcaaagtgttccagaatactgaaggtatggacaggacagacaggccagtctagggagaaagggagatgcagctgtgaaggggatcgttgcccaccaggactgtggtggccaagtgaatgcaggaaccgggcccggaattccggctctcggctaaaatctcagctgcctctggaaaggctcaaccatactcagtgccaactcagactctgttgctgtggtgtcaacatggatggatcatctgtaccttgtatttttagcagaattcatgctcagatttctttgttctgaatccttgctttgtgctagacacaaagcatacatgtccttctaaaattaatatgatcactataatctcctgtgtgcagaattcagaaatagacctttgaaaccatttgcattgtgagtgcagatccatgactggggctagtgcagcaatgaaacagaattccagaaacagtgtgttctttttattatgggaaaatacagataaaaatggccactgatgaacatgaaagttagcactttcccaacacagtgtacacttgcaaccttgttttggatttctcatacaccaagactgtgaaacacaaatttcaagaatgtgttcaaatgtgtgtgtgtgtgtgtgtgtgtgtgtgtgtgtgtgtgtatgtgtgtgtgtgtgtgtgtgcttgtgtgtttctgtcagtggtatgagtgatatgtatgcatgtgtgtatgtatatgtatgtatgtatgtatgtatgtacgtacatatgtatgtatgtatgtatgtatgtatgtatgtatatgtgtgtgtgtgtttgtgtgtgtgtgtgtttgtgtgtgtgtgtgtggtaagtgtggtatgtgtgtatgcatttgtctatatgtgtatctgtgtgtctatgtgtttctgtcagtggaatgagtggcatgtgtgcatgtgtatgtatgtggatatgtgtgttgtgtttatgtgcttgtgtataagaggtaagtgtggtgtgtgtgcatgtgtctctgtgtgtgtttgtctgtgtacctctttgtataagtacctgtgtttgtatgtgggaatatgtatattgaggcattgctgtgttagtatgtttatagaaaagaagacagtctgagatgtcttcctcaatacctctccacttatatcttggatagacaaaagtaatgacaaaaaattgctggtgtgtatatggaaaagggggacacatatccatggatggtagaagtgtaaactgtgcagtcactgtggacatcaatatgcaggttcttcacaaatgtagatataaagctactatagttataccc

SEQ ID NO: 8 - protein, 926 aa, Mus musculus
MDMFPLTWVFLALYFSGHEVRSQQDPPCGGRPNSKDAGYITSPGYPQDYPSHQNCEWIVYAPEPNQKIVLNFNPHFEIEKHDCKYDFIEIRDGDSESADLLGKHCGNIAPPTIISSGSVLYIKFTSDYARQGAGFSLRYEIFKTGSEDCSKNFTSPNGTIESPGFPEKYPHNLDCTFTILAKPRMEIILQFLTFDLEHDPLQVGEGDCKYDWLDIWDGIPHVGPLIGKYCGTKTPSKLRSSTGILSLTFHTDMAVAKDGFSARYYLIHQEPPENFQCNVPLGMESGRIANEQISASSTFSDGRWTPQQSRLHGDDNGWTPNLDSNKEYLQVDLRFLTMLTAIATQGAISRETQKGYYVKSYKLEVSTNGEDWMVYRHGKNHKIFQANNDATEVVLNKLHMPLLTRFIRIRPQTWHLGIALRLELFGCRVTDAPCSNMLGMLSGLIADTQISASSTREYLWSPSAARLVSSRSGWFPRNPQAQPGEEWLQVDLGTPKTVKGVIIQGARGGDSITAVEARAFVRKFKVSYSLNGKDWEYIQDPRTQQTKLFEGNMHYDTPDIRRFDPVPAQYVRVYPERWSPAGIGMRLEVLGCDWTDSKPTVETLGPTVKSEETTTPYPMDEDATECGENCSFEDDKDLQLPSGFNCNFDFPEETCGWVYDHAKWLRSTWISSANPNDRTFPDDKNFLKLQSDGRREGQYGRLISPPVHLPRSPVCMEFQYQAMGGHGVALQVVREASQESKLLWVIREDQGSEWKHGRIILPSYDMEYQIVFEGVIGKGRSGEISGDDIRISTDVPLENCMEPISAFAVDIPETHGGEGYEDEIDDEYEGDWSNSSSSTSGAGDPSSGKEKSWLYTLDPILITIIAMSSLGVLLGATCAGLLLYCTCSYSGLSSRSCTTLENYNFELYDGLKHKVKINHQKCCSEA

SEQ ID NO: 9 - DNA, 2530 bp, Homo sapiens
  CDS (16)..(2331)
ggaattccctgcagcatgggctggttaactaggattgtctgtcttttctggggagtattacttacagcaagagcaaactatcagaatgggaagaacaatgtgccaaggctgaaattatcctacaaagaaatgttggaatccaacaatgtgatcactttcaatggcttggccaacagctccagttatcataccttccttttggatgaggaacggagtaggctgtatgttggagcaaaggatcacatattttcattcgacctggttaatatcaaggattttcaaaagattgtgtggccagtatcttacaccagaagagatgaatgcaagtgggctggaaaagacatcctgaaagaatgtgctaatttcatcaaggtacttaaggcatataatcagactcacttgtacgcctgtggaacgggggcttttcatccaatttgcacctacattgaaattggacatcatcctgaggacaatatttttaagctggagaactcacattttgaaaacggccgtgggaagagtccatatgaccctaagctgctgacagcatcccttttaatagatggagaattatactctggaactgcagctgattttatggggcgagactttgctatcttccgaactcttgggcaccaccacccaatcaggacagagcagcatgattccaggtggctcaatgatccaaagttcattagtgcccacctcatctcagagagtgacaatcctgaagatgacaaagtatactttttcttccgtgaaaatgcaatagatggagaacactctggaaaagctactcacgctagaataggtcagatatgcaagaatgactttggagggcacagaagtctggtgaataaatggacaacattcctcaaagctcgtctgatttgctcagtgccaggtccaaatggcattgacactcattttgatgaactgcaggatgtattcctaatgaactttaaagatcctaaaaatccagttgtatatggagtgtttacgacttccagtaacattttcaagggatcagccgtgtgtatgtatagcatgagtgatgtgagaagggtgttccttggtccatatgcccacagggatggacccaactatcaatgggtgccttatcaaggaagagtcccctatccacggccaggaacttgtcccagcaaaacatttggtggttttgactctacaaaggaccttcctgatgatgttataacctttgcaagaagtcatccagccatgtacaatccagtgtttcctatgaacaatcgcccaatagtgatcaaaacggatgtaaattatcaatttacacaaattgtcgtagaccgagtggatgcagaagatggacagtatgatgttatgtttatcggaacagatgttgggaccgttcttaaagtagtttcaattcctaaggagacttggtatgatttagaagaggttctgctggaagaaatgacagtttttcgggaaccgactgctatttcagcaatggagctttccactaagcagcaacaactatatattggttcaacggctggggttgcccagctccctttacaccggtgtgatatttacgggaaagcgtgtgctgagtgttgcctcgcccgagacccttactgtgcttgggatggttctgcatgttctcgctattttcccactgcaaagagacgcacaagacgacaagatataagaaatggagacccactgactcactgttcagacttacaccatgataatcaccatggccacagccctgaagagagaatcatctatggtgtagagaatagtagcacatttttggaatgcagtccgaagtcgcagagagcgctggtctattggcaattccagaggcgaaatgaagagcgaaaagaagagatcagagtggatgatcatatcatcaggacagatcaaggccttctgctacgtagtctacaacagaaggattcaggcaattacctctgccatgcggtggaacatgggttcatacaaactcttcttaaggtaaccctggaagtcattgacacagagcatttggaagaacttcttcataaagatgatgatggagatggctctaagaccaaagaaatgtccaatagcatgacacctagccagaaggtctggtacagagacttcatgcagctcatcaaccaccccaatctcaacacgatggatgagttctgtgaacaagtttggaaaagggaccgaaaacaacgtcggcaaaggccaggacataccccagggaacagtaacaaatggaagcacttacaagaaaataagaaaggtagaaacaggaggacccacgaatttgagagggcacccaggagtgtctgagctgcattacctctagaaacctcaaacaagtagaaacttgcctagacaataactggaaaaacaaatgcaatatacatgaacttttttcatggcattatgtggatgtttacaatggtgggaaattcagctgagttccaccaattataaattaaatccatgagtaactttcctaataggcttttttttcctaataccaccg

SEQ ID NO: 10 - protein, 771 aa, Homo sapiens [sequence omitted]

SEQ ID NO: 11 - DNA, 2919 bp, Homo sapiens
  CDS (236)..(2485)
tctgtgattgtggccaggcggggcaccctcggaggggagggttcggaagtggaatgcgaccccccagcctctttcccctaggggctgtaatctgatccctggggactccccccctagcctcccgccctcgccctcactgctgactcctcttccagatcctggggcagagtccagggcagctcaaggctcctccacacacacacccgctgaaccctgagcaccctgagctgctgagatggggcgggccggggctgccgccgtgatcccgggcctggccctgctctgggcagtggggctggggagtgccgcccccagccccccacgccttcggctctccttccaagagctccaggcctggcatggtctccagactttcagcctggagcgaacctgctgctaccaggccttgctggtggatgaggagcgtggacgcctgtttgtgggtgccgagaaccatgtggcctccctcaacctggacaacatcagcaagcgggccaagaagctggcctggccggcccctgtggaatggcgagaggagtgcaactgggcagggaaggacattggtactgagtgcatgaacttcgtgaagttgctgcatgcctacaaccgcacccatttgctggcctgtggcacgggagccttccacccaacctgtgcctttgtggaagtgggccaccgggcagaggagcccgtcctccggctggacccaggaaggatagaggatggcaaggggaagagtccttatgaccccaggcatcgggctgcctccgtgctggtgggggaggagctatactcaggggtggcagcagacctcatgggacgagactttaccatctttcgcagcctagggcaacgtccaagtctccgaacagagccacacgactcccgctggctcaatgagcccaagtttgtcaaggtattttggatcccggagagcgagaacccagacgacgacaaaatctacttcttctttcgtgagacggcggtagaggcggcgccggcactgggacgcctgtccgtgtcccgcgttggccagatctgccggaacgacgtgggcggccagcgcagcctggtcaacaagtggacgacgttcctgaaggcgcggctggtgtgctcggtgcccggcgtcgagggcgacacccacttcgatcagctccaggatgtgtttctgttgtcctcgcgggaccaccggaccccgctgctctatgccgtcttctccacgtccagcagcatcttccagggctctgcggtgtgcgtgtacagcatgaacgacgtgcgccgggccttcttgggaccctttgcacacaaggaggggcccatgcaccagtgggtgtcataccagggtcgcgtcccctacccgcggccaggcatgtgccccagcaagacctttggcaccttcagttccaccaaggacttcccagacgatgtcatccagtttgcgcggaaccaccccctcatgtacaactctgtcctgcccactggggggcgccctcttttcctacaagttggagccaattacaccttcactcaaattgccgcggaccgggttgcagccgctgacggacactatgacgtcctcttcattggcacagacgttggcacggtgctgaaggtgatctcggtccccaagggcagtaggcccagcgcagaggggctgctcctggaggagctgcacgtgtttgaggactcggccgctgtcaccagcatgcaaatttcttccaagaggcaccagctgtacgtagcctcgcggagcgcggtggcccagatcgcgttgcaccgctgcgctgcccacggccgcgtctgcaccgaatgctgtctggcgcgtgacccctactgcgcctgggacggggtcgcgtgcacgcgcttccagcccagtgccaagaggcggttccggcggcaagacgtaaggaatggcgaccccagcacgttgtgctccggagactcgtctcgtcccgcgctgctggaacacaaggtgttcggcgtggagggcagcagcgcctttctggagtgtgagccccgctcgctgcaggcgcgcgtggagtggactttccagcgcgcaggggtgacagcccacacccaggtgctggcagaggagcgcaccgagcgcaccgcccggggactactgctgcgcaggctgcggcgccgggactcgggcgtgtacttgtgcgccgccgtcgagcagggctttacgcaaccgctgcgtcgcctgtcgctgcacgtgttgagtgctacgcaggccgaacgactggcgcgggccgaggaggctgcgcccgccgcgccgccgggccccaaactctggtaccgggactttctgcagctggtggagccgggcggaggtggcagcgcgaactccctgcgcatgtgccgcccgcagcctgcgctgcagtcactgcccctggagtcgcggagaaagggccgtaaccggaggacccacgcccctgagcctcgcgctgagcgggggccgcgcagcgcaacgcactggtgaccagactgtccccacgccgggaaccaagcaggagacgacaggcgagagaggagccagacagaccctgaaaagaaggacgggttggggccgggcacattgggggtcaccggccgatggagacaccaaccgacaggccctggctgagggcagctgcgcgggcttatttattaacaggataacccttgaatgtagcagccccgggagggcggcacaggtcgggcgcaggattcagccggagggaagggacggggaagccgagctccagagcaacgaccagggccgaggaggtgcctggagtgcccaccctgggagacagaccccacctccttgggtagtgagcagtgagcagaaagctgtgaacaggctgggctgctggaggtggggcgaggcaggccgactgtactaaagtaacgcaataaacgcattatcagcca

SEQ ID NO: 12 - protein, 749 aa, Homo sapiens
MGRAGAAAVIPGLALLWAVGLGSAAPSPPRLRLSFQELQAWHGLQTFSLERTCCYQALLVDEERGRLFVGAENHVASLNLDNISKRAKKLAWPAPVEWREECNWAGKDIGTECMNFVKLLHAYNRTHLLACGTGAFHPTCAFVEVGHRAEEPVLRLDPGRIEDGKGKSPYDPRHRAASVLVGEELYSGVAADLMGRDFTIFRSLGQRPSLRTEPHDSRWLNEPKFVKVFWIPESENPDDDKIYFFFRETAVEAAPALGRLSVSRVGQICRNDVGGQRSLVNKWTTFLKARLVCSVPGVEGDTHFDQLQDVFLLSSRDHRTPLLYAVFSTSSSIFQGSAVCVYSMNDVRRAFLGPFAHKEGPMHQWVSYQGRVPYPRPGMCPSKTFGTFSSTKDFPDDVIQFARNHPLMYNSVLPTGGRPLFLQVGANYTFTQIAADRVAAADGHYDVLFIGTDVGTVLKVISVPKGSRPSAEGLLLEELHVFEDSAAVTSMQISSKRHQLYVASRSAVAQIALHRCAAHGRVCTECCLARDPYCAWDGVACTRFQPSAKRRFRRQDVRNGDPSTLCSGDSSRPALLEHKVFGVEGSSAFLECEPRSLQARVEWTFQRAGVTAHTQVLAEERTERTARGLLLRRLRRRDSGVYLCAAVEQGFTQPLRRLSLHVLSATQAERLARAEEAAPAAPPGPKLWYRDFLQLVEPGGGGSANSLRMCRPQPALQSLPLESRRKGRNRRTHAPEPRAERGPRSATHW

SEQ ID NO: 13 - DNA, 5177 bp, Homo sapiens
  CDS (563)..(2818)
ggactgcgaaaggagcagggttgcggagctagggctccagcctgcggccgcgcattcttgcgtctggccagccgcgagctctaagggtcggccccgcccggtccgcccccgcggctccctgccaggctctcgcgggcgcgctcggggtggggcctcgcggctggcggagatgcggccggggctgcgcggtggtgatgcgagcctgctgggcggcgcgccggggcagccggagccgcgcgccgcggcgctgtaatcggacaccaagagcgctcgcccccggcctccggccactttccattcactccgaggtgcttgattgagcgacgcggagaagagctccgggtgccgcggcactgcagcgctgagattcctttacaaagaaactcagaggaccgggaagaaagaatttcacctttgcgacgtgctagaaaataaggtcgtctgggaaaaggactggagacacaagcgcatccaaccccggtagcaaactgatgacttttccgtgctgatttctttcaacctcggtattttcccttggatattaacttgcatatctgaagaaatggcattccggacaatttgcgtgttggttggagtatttatttgttctatctgtgtgaaaggatcttcccagccccaagcaagagtttatttaacatttgatgaacttcgagaaaccaagacctctgaatacttcagcctttcccaccatcctttagactacaggattttattaatggatgaagatcaggaccggatatatgtgggaagcaaagatcacattctttccctgaatattaacaatataagtcaagaagctttgagtgttttctggccagcatctacaatcaaagttgaagaatgcaaaatggctggcaaagatcccacacacggctgtgggaactttgtccgtgtaattcagactttcaatcgcacacatttgtatgtctgtgggagtggcgctttcagtcctgtctgtacttacttgaacagagggaggagatcagaggaccaagttttcatgattgactccaagtgtgaatctggaaaaggacgctgctctttcaaccccaacgtgaacacggtgtctgttatgatcaatgaggagcttttctctggaatgtatatagatttcatggggacagatgctgctatttttcgaagtttaaccaagaggaatgcggtcagaactgatcaacataattccaaatggctaagtgaacctatgtttgtagatgcacatgtcatcccagatggtactgatccaaatgatgctaaggtgtacttcttcttcaaagaaaaactgactgacaataacaggagcacgaaacagattcattccatgattgctcgaatatgtcctaatgacactggtggactgcgtagccttgtcaacaagtggaccactttcttaaaggcgaggctggtgtgctcggtaacagatgaagacggcccagaaacacactttgatgaattagaggatgtgtttctgctggaaactgataacccgaggacaacactagtgtatggcatttttacaacatcaagctcagttttcaaaggatcagccgtgtgtgtgtatcatttatctgatatacagactgtgtttaatgggccttttgcccacaaagaagggcccaatcatcagctgatttcctatcagggcagaattccatatcctcgccctggaacttgtccaggaggagcatttacacccaatatgcgaaccaccaaggagttcccagatgatgttgtcacttttattcggaaccatcctctcatgtacaattccatctacccaatccacaaaaggcctttgattgttcgtattggcactgactacaagtacacaaagatagctgtggatcgagtgaacgctgctgatgggagataccatgtcctgtttctcggaacagatcggggtactgtgcaaaaagtggttgttcttcctactaacaactctgtcagtggcgagctcattctggaggagctggaagtctttaagaatcatgctcctataacaacaatgaaaatttcatctaaaaagcaacagttgtatgtgagttccaatgaaggggtttcccaagtatctctgcaccgctgccacatctatggtacagcctgtgctgactgctgcctggcgcgggacccttattgcgcctgggatggccattcctgttccagattctacccaactgggaaacggaggagccgaagacaagatgtgagacatggaaacccactgactcaatgcagaggatttaatctaaaagcatacagaaatgcagctgaaattgtgcagtatggagtaaaaaataacaccacttttctggagtgtgcccccaagtctccgcaggcatctatcaagtggctgttacagaaagacaaagacaggaggaaagaggttaagctgaatgaacgaataatagccacttcacagggactcctgatccgctctgttcagggttctgaccaaggactttatcactgcattgctacagaaaatagtttcaagcagaccatagccaagatcaacttcaaagttttagattcagaaatggtggctgttgtgacggacaaatggtccccgtggacctgggccagctctgtgagggctttacccttccacccgaaggacatcatgggggcattcagccactcagaaatgcagatgattaaccaatactgcaaagacactcggcagcaacatcagcagggagatgaatcacagaaaatgagaggggactatggcaagttaaaggccctcatcaatagtcggaaaagtagaaacaggaggaatcagttgccagagtcataatattttcttatgtgggtcttatgcttccattaacaaatgctctgtcttcaatgatcaaattttgagcaaagaaacttgtgctttaccaaggggaattactgaaaaaggtgattactcctgaagtgagttttacacgaactgaaatgagcatgcattttcttgtatgatagtgactagcactagacatgtcatggtcctcatggtgcatataaatatatttaacttaacccagattttatttatatctttattcaccttttcttcaaaatcgatatggtggctgcaaaactagaattgttgcatccctcaattgaatgagggccatatccctgtggtattcctttcctgctttggggctttagaattctaattgtcagtgattttgtatatgaaaacaagttccaaatccacagcttttacgtagtaaaagtcataaatgcatatgacagaatggctatcaaaagaaatagaaaaggaagacggcatttaaagttgtataaaaacacgagttattcataaagagaaaatgatgagtttttatggttccaatgaaatatcttcccctttttttaagattgtaaaaataatcagttactggtatctgtcactgacctttgtttccttattcaggaagataaaaatcagtaacctaccccatgaagatatttggtgggagttatatcagtgaagcagtttggtttatattcttatgttatcaccttccaaacaaaagcacttactttttttggaagttatttaatttattttagactcaaagaatataatcttgcactactcagttattactgtttgttctcttattccctagtctgtgtggcaaattaaacaatataagaaggaaaaatttgaagtattagacttctaaataaggggtgaaatcatcagaaagaaaaatcaaagtagaaactactaattttttaagaggaatttataacaaatatggctagttttcaacttcagtactcaaattcaatgattcttccttttattaaaaccagtctcagatatcatactgatttttaagtcaacactatatattttatgatcttttcagtgtgatggcaaggtgcttgttatgtctagaaagtaagaaaacaatatgaggagacattctgtctttcaaaaggtaatggtacatacgttcactggtctctaagtgtaaaagtagtaaattttgtgatgaataaaataattatctcctaattgtatgttagaataattttattagaataatttcatactgaaattattttctccaaataaaaattagatggaaaaatgtgaaaaaaattattcatgctctcatatatattttaaaaacactacttttgcttttttatttaccttttaagacattttcatgcttccaggtaaaaacagatattgtaccatgtacctaatccaaatatcatataaacattttatttatagttaataatctatgatgaaggtaattaaagtagattatggcctttttaagtattgcagtctaaaacttcaaaaactaaaatcattgtcaaaattaatatgattattaatcagaatatcagatatgattcactatttaaactatgataaattatgataatatatgaggaggcctcgctatagcaaaaatagttaaaatgctgacataacaccaaacttcattttttaaaaaatctgttgttccaaatgtgtataattttaaagtaatttctaaagcagtttattataatggtttgcctgcttaaaaggtataattaaacttcttttctcttctacattgacacacagaaatgtgtcaatgtaaagccaaaaccatcttctgtgtttatggccaatctattctcaaagttaaaagtaaaattgtttcagagtcacagttccctttatttcacataagcccaaactgatagacagtaacggtgtttagttttatactatatttgtgctatttaattctttctattttcacaattattaaattgtgtacactttcattacttttaaaaatgtagaaattcttcatgaacataactctgctgaatgtaaaagaaaattttttttcaaaaatgctgttaatgtatactactggtggttgattggttttattttatgtagcttgacaattcagtgacttaatatctattccatttgtattgtacataaaattttctagaaatacacttttttccaaagtgtaagtttgtgaatagattttagcatgatgaaactgtcataatggtgaatgttcaatctgtgtaagaaaacaaactaaatgtagttgtcacactaaaatttaattggatattgatgaaatcattggcctggcaaaataaaacatgttgaattcccc

SEQ ID NO: 14 - protein, 751 aa, Homo sapiens
MAFRTICVLVGVFICSICVKGSSQPQARVYLTFDELRETKTSEYFSLSHHPLDYRILLMDEDQDRIYVGSKDHILSLNINNISQEALSVFWPASTIKVEECKMAGKDPTHGCGNFVRVIQTFNRTHLYVCGSGAFSPVCTYLNRGRRSEDQVFMIDSKCESGKGRCSFNPNVNTVSVMINEELFSGMYIDFMGTDAAIFRSLTKRNAVRTDQHNSKWLSEPMFVDAHVIPDGTDPNDAKVYFFFKEKLTDNNRSTKQIHSMIARICPNDTGGLRSLVNKWTTFLKARLVCSVTDEDGPETHFDELEDVFLLETDNPRTTLVYGIFTTSSSVFKGSAVCVYHLSDIQTVFNGPFAHKEGPNHQLISYQGRIPYPRPGTCPGGAFTPNMRTTKEFPDDVVTFIRNHPLMYNSIYPIHKRPLIVRIGTDYKYTKIAVDRVNAADGRYHVLFLGTDRGTVQKVVVLPTNNSVSGELILEELEVFKNHAPITTMKISSKKQQLYVSSNEGVSQVSLHRCHIYGTACADCCLARDPYCAWDGHSCSRFYPTGKRRSRRQDVRHGNPLTQCRGFNLKAYRNAAEIVQYGVKNNTTFLECAPKSPQASIKWLLQKDKDRRKEVKLNERIIATSQGLLIRSVQGSDQGLYHCIATENSFKQTIAKINFKVLDSEMVAVVTDKWSPWTWASSVRALPFHPKDIMGAFSHSEMQMINQYCKDTRQQHQQGDESQKMRGDYGKLKALINSRKSRNRRNQLPES

SEQ ID NO: 15 - DNA, 6474 bp, Homo sapiens
  CDS (467)..(2794)
gtttggcaagtcagtgcaagaggctgacttctgagaggcttccaggagcccgaagagaggacctccacgggagaagggagtgcgtgtgctcggttttttttttttctctcttttttttttttttttctgaatgaacagctttgcccaagtgactgaaaaatacagcttcttcctgaatctaccggcgtagttgctgaagagcgctctagacaggacatggctctgaagactcactctttggaatgtcctcttgctcccggcttataaacaactgtcccgaggaaagaaaggttttacatagccaaatacagcctgacaaatggcacttcggaactgtgctttctgatgacaacgcgttcgatttctgacaaagcctctcgcacgctgcccctggagggaagtcctaagtaaaactcagaccctccttaaagtgaggagcgagggcttggacggtgaacacggcagcatggcatccgcggggcacattatcaccttgctcctgtggggttacttactggagctttggacaggaggtcatacagctgatactacccacccccggttacgcctgtcacataaagagctcttgaatctgaacagaacatcaatatttcatagcccttttggatttcttgatctccatacaatgctgctggatgaatatcaagagaggctcttcgtgggaggcagggaccttgtatattccctcagcttggagagaatcagtgacggctataaagagatacactggccgagtacagctctaaaaatggaagaatgcataatgaagggaaaagatgcgggtgaatgtgcaaattatgttcgggttttgcatcactataacaggacacaccttctgacctgtggtactggagcttttgatccagtttgtgccttcatcagagttggatatcatttggaggatcctctgtttcacctggaatcacccagatctgagagaggaaggggcagatgtccttttgaccccagctcctccttcatctccactttaattggtagtgaattgtttgctggactctacagtgactactggagcagagacgctgcgatcttccgcagcatggggcgactggcccatatccgcactgagcatgacgatgagcgtctgttgaaagaaccaaaatttgtaggttcatacatgattcctgacaatgaagacagagatgacaacaaagtatatttcttttttactgagaaggcactggaggcagaaaacaatgctcacgcaatttacaccagggtcgggcgactctgtgtgaatgatgtaggagggcagagaatactggtgaataagtggagcactttcctaaaagcgagactcgtttgctcagtaccaggaatgaatggaattgacacatattttgatgaattagaggacgtttttttgctacctaccagagatcataagaatccagtgatatttggactctttaacactaccagtaatatttttcgagggcatgctatatgtgtctatcacatgtctagcattcgggcagccttcaacggaccatatgcacataaggaaggacctgaataccactggtcagtctatgaaggaaaagtcccttatccaaggcctggttcttgtgccagcaaagtaaatggagggagatacggaaccaccaaggactatcctgatgatgccatccgatttgcaagaagtcatccactaatgtaccaggccataaaacctgcccataaaaaaccaatattggtaaaaacagatggaaaatataacctgaaacaaatagcagtagatcgagtggaagctgaggatggccaatatgacgtcttgtttattgggacagataatggaattgtgctgaaagtaatcacaatttacaaccaagaaatggaatcaatggaagaagtaattctagaagaacttcagatattcaaggatccagttcctattatttctatggagatttcttcaaaacggcaacagctgtatattggatctgcttctgctgtggctcaagtcagattccatcactgtgacatgtatggaagtgcttgtgctgactgctgcctggctcgagacccttactgtgcctgggatggcatatcctgctcccggtattacccaacaggcacacatgcaaaaaggcgtttccggagacaagatgttcgacatggaaatgcagctcagcagtgctttggacaacagtttgttggggatgctttggataagactgaagaacatctggcttatggcatagagaacaacagtactttgctggaatgtaccccacgatctttacaagcgaaagttatctggtttgtacagaaaggacgtgagacaagaaaagaggaggtgaagacagatgacagagtggttaagatggaccttggtttactcttcctaaggttacacaaatcagatgctgggacctatttttgccagacagtagagcatagctttgtccatacggtccgtaaaatcaccttggaggtagtggaagaggagaaagtcgaggatatgtttaacaaggacgatgaggaggacaggcatcacaggatgccttgtcctgctcagagtagcatctcgcagggagcaaaaccatggtacaaggaattcttgcagctgatcggttatagcaacttccagagagtggaagaatactgcgagaaagtatggtgcacagatagaaagaggaaaaagcttaaaatgtcaccctccaagtggaagtatgccaaccctcaggaaaagaagctccgttccaaacctgagcattaccgcctgcccaggcacacgctggactcctgatggggtgagactatctactgtcttttgaagaatttatatttggaaagtaaaaaagtaaaaaaataaatcatccaacttctttgcattacttaaaagagatttctgtaatacaggaatgactatgaaggtgttataataaattattctacatactcatttgactggataaactttacataaaattaactaattttttaaataaatgcattgcttaatggtttctcattatgtttatcaaaaaacaactgtagctgttattttcagtacttggctgcttttctgtgaaaattattattttacttttggaagacaagattattagaatattgaagaaaaattggagacttataatcatggtaaatataaaactaaatatgttttaatatttctgaatttttcttttccatcacaatgtaagatatgcagaatacaagatactttggcattctcatgtgaactttctgtactctttaaggattattttattagtgttgtttaagccatgagtgttaagtagcaggtgtgttgtgagtgctgtaacccatgaaaggaaaaatgtcattctgaggcttgtgcccttcgtaaaatattcattaaagtacattcacactatttttgctttataacacagtctttaattttcactcactgtggaaataaaaactaaggtaacttctcagaaagatatcaaatctcagaaagaatgtcaaatcagatgaagttatagttaggattctaactactgtaaaagatttttgcttccctcttgtggtaaaaaaaattatattctcacacatttcttttttctctacagacggatatctgtttaggaaagatttgaaagcagattatcagtaggtacatggatacatcaagttcatttgcagaaacaaataactgaaataaaaaacatgttaatccttgtatcatactttaatatgaaagtattgtttatagataatttatctcacaagtcaaaaatgaagattttgcagcactgaaaatctattaaagctccaaattttaagtttctaaataatcttcgctgaaatctaaaatatactataacaaccgtgttttatttgtgaaaaaaatattaaagtgatttgctctcaaatatcaaattttcttctctcttttatattaagagacagaaaattgtttcatgagttcacttaactactgagatattcagagcatttttacctctctcttaaatgttataaaaaacaattgtatttttaagaatgtttatttatcaaagtctttccttcttctattaaatatttagcaattacctttctaaaatatgaaattttgtaagatgttttcacctaaataaaaattgaaagcaagtggattacacaggagaaccattatgaacatttatttagatattaatcttaaacagtgtttatttcagttttcaaagttagcttataggttatacatttaagttaaagtgctcataatcacttgcaatttcattgtaaaatgaacaaatacataaatattttaagaaaaatttaagtttattcagataagtcaccatgcttcaaaagatctaagaaatgcaaatatactgaaaattgacatcctctgaaaattccacttgctatttacccaagaatccactggaggtcattactgccattaaataataactgaaaagactatgtagtgaaatgtatttttaaaaactatattcagtaaaagcctgctcaatttggagaaatagaaccacaaacacagatcacaggggccttacaaagtttatgtctgaacaaataagtcaattaagtacactttattgaaaattgccttccattaacacacaagaaagaaagcaggattttctcctgtatctgaattttaaaattaaaaaggcagataagacataaatagttatcattttaattgcaataacacagacaagtagttaatgatgataacaatggtgtaacttgtaaactaaatatttggtaactgaagcaataggcagaggaaaatagcttttctatgacacaagtcataagaagtccatatactgaagagcgtttgattaaaataaagtgactattaaccagaaaagaaacattttacataaaatgctaaaatttattataggaaaataaatcaaacccaaagaaagtttattcaatgctaatttgaaagaaaattgataagaaaactttgagggcccaagtccacaatttggtgagaccactaaattttacatataattatacacacacatatgtacatatatatgtatataatcttgcttcccgcctgtttatggcagtactgaagagaaatgggaaagaagagggagggagagagaaagacgaagggagagagaaagcagtttccaaggatatgtttcatgtcccaccattttctcagtttctccctctctctcccaacacacacacacacacacccctcacatactataaaataaatcttcactgccctatcaaaatacaaataaatcaatctatgctgttctgtccttcttgagaatctaaaacataccacaaaaatacatccccagtcttttgttctgtctgaggttagaattaattcaaattcagaatctgttgtgagaaatgcccaggctttaaaaattaaaaatggatggatcttctctgaactcagggagggcacatacttagatacctacaagacttggaggaattaagagttcacccttcatctcaccaaattttccccatttttctctttcttgtagaaggagagaaaccatgctctctagcaacattgagcaaaaatcataaccactcatctaatttctaagaggcacctccatcgagggccggtctcctgcttctttagacctcttctatctttgttacaggagaggacctgtggatagacttagttttgacataaaacaatgcccattcacctcctccttcagcacaacgtcacccattgggcaagagatccagatttgttaacaaaaaagattttacttcgtgattccacgtctataattctatattgctaattttttcttttgtgtgaattactgaatatttcagagcaaagctatcaacttggagaaacagggattaaaaataaggataaacactaataagagctctagaaaaaagggaacagaaagtctgcctgtttagtaagtggcaattccatacatattttagagttttttctatctaaaattagttaaatacttagaatgtttgtaatgagtgttcgatatttgctataggttttagggttttgtaaatcttcatagtaattataaacatttgtaaaatttgtaaaatactataagtcattttgagtgttggtgttaagcatgaaacaaacagcagctgttgtccttaaaaatgaattgacctggccgggcgcggtggctcacgcctgtaatcccagcactttgggaggccgaggcgggtggatcatgaggtcaggagatggagaccatcctggctaacaaggtgaaaccccgtctctactaaaaatacaaaaaattagccgggcgcggtggcgggcgcctgtagtcccagctacttgggaggctgaggcaggagaatggcgtgaacccgggaagcggagcttgcagtgagccgagattgcgccactgcagtccgcagtccggcctgggcgacagagcgagactccgtctc

SEQ ID NO: 16 - protein, 775 aa, Homo sapiens
MASAGHIITLLLWGYLLELWTGGHTADTTHPRLRLSHKELLNLNRTSIFHSPFGFLDLHTMLLDEYQERLFVGGRDLVYSLSLERISDGYKEIHWPSTALKMEECIMKGKDAGECANYVRVLHHYNRTHLLTCGTGAFDPVCAFIRVGYHLEDPLFHLESPRSERGRGRCPFDPSSSFISTLIGSELFAGLYSDYWSRDAAIFRSMGRLAHIRTEHDDERLLKEPKFVGSYMIPDNEDRDDNKVYFFFTEKALEAENNAHAIYTRVGRLCVNDVGGQRILVNKWSTFLKARLVCSVPGMNGIDTYFDELEDVFLLPTRDHKNPVIFGLFNTTSNIFRGHAICVYHMSSIRAAFNGPYAHKEGPEYHWSVYEGKVPYPRPGSCASKVNGGRYGTTKDYPDDAIRFARSHPLMYQAIKPAHKKPILVKTDGKYNLKQIAVDRVEAEDGQYDVLFIGTDNGIVLKVITIYNQEMESMEEVILEELQIFKDPVPIISMEISSKRQQLYIGSASAVAQVRFHHCDMYGSACADCCLARDPYCAWDGISCSRYYPTGTHAKRRFRRQDVRHGNAAQQCFGQQFVGDALDKTEEHLAYGIENNSTLLECTPRSLQAKVIWFVQKGRETRKEEVKTDDRVVKMDLGLLFLRLHKSDAGTYFCQTVEHSFVHTVRKITLEVVEEEKVEDMFNKDDEEDRHHRMPCPAQSSISQGAKPWYKEFLQLIGYSNFQRVEEYCEKVWCTDRKRKKLKMSPSKWKYANPQEKKLRSKPEHYRLPRHTLDS

SEQ ID NO: 17 - DNA, 2719 bp, Homo sapiens
  CDS (79)..(2436)
cggggcccaggccccgccgctgcggaagaggtttctagagagtggagcctgcttcctgggccctaggcccctcccacaatgcttgtcgccggtcttcttctctgggcttccctactgaccggggcctggccatccttccccacccaggaccacctcccggccacgccccgggtccggctctcattcaaagagctgaaggccacaggcaccgcccacttcttcaacttcctgctcaacacaaccgactaccgaatcttgctcaaggacgaggaccacgaccgcatgtacgtgggcagcaaggactacgtgctgtccctggacctgcacgacatcaaccgcgagcccctcattatacactgggcagcctccccacagcgcatcgaggaatgcgtgctctcaggcaaggatgtcaacggcgagtgtgggaacttcgtcaggctcatccagccctggaaccgaacacacctgtatgtgtgcgggacaggtgcctacaaccccatgtgcacctatgtgaaccgcggacgccgcgcccaggccacaccatggacccagactcaggcggtcagaggccgcggcagcagagccacggatggtgccctccgcccgatgcccacagccccacgccaggattacatcttctacctggagcctgagcgactcgagtcagggaagggcaagtgtccgtacgatcccaagctggacacagcatcggccctcatcaatgaggagctctatgctggtgtgtacatcgattttatgggcactgatgcagccatcttccgcacacttggaaagcagacagccatgcgcacggatcagtacaactcccggtggctgaacgacccgtcgttcatccatgctgagctcattcctgacagtgcggagcgcaatgatgataagctttacttcttcttccgtgagcggtcggcagaggcgccgcagagccccgcggtgtacgcccgcatcgggcgcatttgcctgaacgatgacggtggtcactgttgcctggtcaacaagtggagcacattcctgaaggcgcggctcgtctgctctgtcccgggcgaggatggcattgagactcactttgatgagctccaggacgtgtttgtccagcagacccaggacgtgaggaaccctgtcatttacgctgtctttacctcctctggctccgtgttccgaggctctgccgtgtgtgtctactccatggctgatattcgcatggtcttcaacgggccctttgcccacaaagaggggcccaactaccagtggatgcccttctcagggaagatgccctacccacggccgggcacgtgccctggtggaaccttcacgccatctatgaagtccaccaaggattatcctgatgaggtgatcaacttcatgcgcagccacccactcatgtaccaggccgtgtaccctctgcagcggcggcccctggtagtccgcacaggtgctccctaccgccttaccactattgccgtggaccaggtggatgcaggcgacgggcgctatgaggtgcttttcctgggcacagaccgcgggacagtgcagaaggtcattgtgctgcccaaggatgaccaggagatggaggagctcatgctggaggaggtggaggtcttcaaggatccagcacccgtcaagaccatgaccatctcttctaagaggcaacaactctacgtggcgtcagccgtgggtgtcacacacctgagcctgcaccgctgccaggcgtatggggctgcctgtgctgactgctgccttgcccgggacccttactgtgcctgggatggccaggcctgctcccgctatacagcatcctccaagaggcggagccgccggcaggacgtccggcacggaaaccccatcaggcagtgccgtgggttcaactccaatgccaacaagaatgccgtggagtctgtgcagtatggcgtggccggcagcgcagccttccttgagtgccagccccgctcgccccaagccactgttaagtggctgttccagcgagatcctggtgaccggcgccgagagattcgtgcagaggaccgcttcctgcgcacagagcagggcttgttgctccgtgcactgcagctcagcgatcgtggcctctactcctgcacagccactgagaacaactttaagcacgtcgtcacacgagtgcagctgcatgtactgggccgggacgccgtccatgctgccctcttcccaccactgtccatgagcgccccgccacccccaggcgcaggccccccaacgcctccttaccaggagttagcccagctgctggcccagccagaagtgggcctcatccaccagtactgccagggttactggcgccatgtgccccccagccccagggaggctccaggggcaccccggtctcctgagccccaggaccagaaaaagccccggaaccgccggcaccaccctccggacacatgaggccagctgcctgtgcctgccatgggccaggctaggccttggtcccttttaatataaaagatatatatatatatatatatatatattaaaatatcggggtggggggtgattggaagggagggaggtggccttcccaatgcgcgttattcggggttattgaagaataatattgcaagtgacagccagaagtagactttctgtcctcacaccgaagaacccgagtgagcaggagggagggagagacgcgaagagaccttttttcctttttggagaccttgtccgc

SEQ ID NO: 18 - protein, 785 aa, Homo sapiens
MLVAGLLLWASLLTGAWPSFPTQDHLPATPRVRLSFKELKATGTAHFFNFLLNTTDYRILLKDEDHDRMYVGSKDYVLSLDLHDINREPLIIHWAASPQRIEECVLSGKDVNGECGNFVRLIQPWNRTHLYVCGTGAYNPMCTYVNRGRRAQATPWTQTQAVRGRGSRATDGALRPMPTAPRQDYIFYLEPERLESGKGKCPYDPKLDTASALINEELYAGVYIDFMGTDAAIFRTLGKQTAMRTDQYNSRWLNDPSFIHAELIPDSAERNDDKLYFFFRERSAEAPQSPAVYARIGRICLNDDGGHCCLVNKWSTFLKARLVCSVPGEDGIETHFDELQDVFVQQTQDVRNPVIYAVFTSSGSVFRGSAVCVYSMADIRMVFNGPFAHKEGPNYQWMPFSGKMPYPRPGTCPGGTFTPSMKSTKDYPDEVINFMRSHPLMYQAVYPLQRRPLVVRTGAPYRLTTIAVDQVDAGDGRYEVLFLGTDRGTVQKVIVLPKDDQEMEELMLEEVEVFKDPAPVKTMTISSKRQQLYVASAVGVTHLSLHRCQAYGAACADCCLARDPYCAWDGQACSRYTASSKRRSRRQDVRHGNPIRQCRGFNSNANKNAVESVQYGVAGSAAFLECQPRSPQATVKWLFQRDPGDRRREIRAEDRFLRTEQGLLLRALQLSDRGLYSCTATENNFKHVVTRVQLHVLGRDAVHAALFPPLSMSAPPPPGAGPPTPPYQELAQLLAQPEVGLIHQYCQGYWRHVPPSPREAPGAPRSPEPQDQKKPRNRRHHPPDT

SEQ ID NO: 19 - DNA, 649 bp, Homo sapiens
  CDS (17)..(592)
  misc_feature (17)..(94): Signal peptide
tcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtgagccgggcaggaggaaggagcctccctcagcgtttcgggaaccagatctctcaccagg

SEQ ID NO: 20 - protein, 191 aa, Homo sapiens
  misc_feature (17)..(94): Signal peptide
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 21 - DNA, 755 bp, Homo sapiens
  CDS (5)..(628)
caccatgagccctctgctccgccgcctgctgctcgccgcactcctgcagctggcccccgcccaggcccctgtctcccagcctgatgcccctggccaccagaggaaagtggtgtcatggatagatgtgtatactcgcgctacctgccagccccgggaggtggtggtgcccttgactgtggagctcatgggcaccgtggccaaacagctggtgcccagctgcgtgactgtgcagcgctgtggtggctgctgccctgacgatggcctggagtgtgtgcccactgggcagcaccaagtccggatgcagatcctcatgatccggtacccgagcagtcagctgggggagatgtccctggaagaacacagccagtgtgaatgcagacctaaaaaaaaggacagtgctgtgaagccagacagggctgccactccccaccaccgtccccagccccgttctgttccgggctgggactctgcccccggagcaccctccccagctgacatcacccatcccactccagccccaggcccctctgcccacgctgcacccagcaccaccagcgccctgacccccggacctgccgccgccgctgccgacgccgcagcttcctccgttgccaagggcggggcttagagctcaacccagacacctgcaggtgccggaagctgcgaaggtgacacatggcttttcagactcagcagggtgacttgcctcagaggctatatcccagtgggggaacaaagaggagcctggtaaaaaa

SEQ ID NO: 22 - protein, 207 aa, Homo sapiens
MSPLLRRLLLAALLQLAPAQAPVSQPDAPGHQRKVVSWIDVYTRATCQPREVVVPLTVELMGTVAKQLVPSCVTVQRCGGCCPDDGLECVPTGQHQVRMQILMIRYPSSQLGEMSLEEHSQCECRPKKKDSAVKPDRAATPHHRPQPRSVPGWDSAPGAPSPADITHPTPAPGPSAHAAPSTTSALTPGPAAAAADAAASSVAKGGA

SEQ ID NO: 23 - DNA, 1997 bp, Homo sapiens
  CDS (352)..(1611)
cccgccccgcctctccaaaaagctacaccgacgcggaccgcggcggcgtcctccctcgccctcgcttcacctcgcgggctccgaatgcggggagctcggatgtccggtttcctgtgaggcttttacctgacacccgccgcctttccccggcactggctgggagggcgccctgcaaagttgggaacgcggagccccggacccgctcccgccgcctccggctcgcccagggggggtcgccgggaggagcccgggggagagggaccaggaggggcccgcggcctcgcaggggcgcccgcgcccccacccctgcccccgccagcggaccggtcccccacccccggtccttccaccatgcacttgctgggcttcttctctgtggcgtgttctctgctcgccgctgcgctgctcccgggtcctcgcgaggcgcccgccgccgccgccgccttcgagtccggactcgacctctcggacgcggagcccgacgcgggcgaggccacggcttatgcaagcaaagatctggaggagcagttacggtctgtgtccagtgtagatgaactcatgactgtactctacccagaatattggaaaatgtacaagtgtcagctaaggaaaggaggctggcaacataacagagaacaggccaacctcaactcaaggacagaagagactataaaatttgctgcagcacattataatacagagatcttgaaaagtattgataatgagtggagaaagactcaatgcatgccacgggaggtgtgtatagatgtggggaaggagtttggagtcgcgacaaacaccttctttaaacctccatgtgtgtccgtctacagatgtgggggttgctgcaatagtgaggggctgcagtgcatgaacaccagcacgagctacctcagcaagacgttatttgaaattacagtgcctctctctcaaggccccaaaccagtaacaatcagttttgccaatcacacttcctgccgatgcatgtctaaactggatgtttacagacaagttcattccattattagacgttccctgccagcaacactaccacagtgtcaggcagcgaacaagacctgccccaccaattacatgtggaataatcacatctgcagatgcctggctcaggaagattttatgttttcctcggatgctggagatgactcaacagatggattccatgacatctgtggaccaaacaaggagctggatgaagagacctgtcagtgtgtctgcagagcggggcttcggcctgccagctgtggaccccacaaagaactagacagaaactcatgccagtgtgtctgtaaaaacaaactcttccccagccaatgtggggccaaccgagaatttgatgaaaacacatgccagtgtgtatgtaaaagaacctgccccagaaatcaacccctaaatcctggaaaatgtgcctgtgaatgtacagaaagtccacagaaatgcttgttaaaaggaaagaagttccaccaccaaacatgcagctgttacagacggccatgtacgaaccgccagaaggcttgtgagccaggattttcatatagtgaagaagtgtgtcgttgtgtcccttcatattggaaaagaccacaaatgagctaagattgtactgttttccagttcatcgattttctattatggaaaactgtgttgccacagtagaactgtctgtgaacagagagacccttgtgggtccatgctaacaaagacaaaagtctgtctttcctgaaccatgtggataactttacagaaatggactggagctcatctgcaaaaggcctcttgtaaagactggttttctgccaatgaccaaacagccaagattttcctcttgtgatttctttaaaagaatgactatataatttatttccactaaaaatattgtttctgcattcatttttatagcaacaacaattggtaaaactcactgtgatcaatatttttatatcatgcaaaatatgtttaaaataaaatgaaaattgtattat

SEQ ID NO: 24 - protein, 419 aa, Homo sapiens
MHLLGFFSVACSLLAAALLPGPREAPAAAAAFESGLDLSDAEPDAGEATAYASKDLEEQLRSVSSVDELMTVLYPEYWKMYKCQLRKGGWQHNREQANLNSRTEETIKFAAAHYNTEILKSIDNEWRKTQCMPREVCIDVGKEFGVATNTFFKPPCVSVYRCGGCCNSEGLQCMNTSTSYLSKTLFEITVPLSQGPKPVTISFANHTSCRCMSKLDVYRQVHSIIRRSLPATLPQCQAANKTCPTNYMWNNHICRCLAQEDFMFSSDAGDDSTDGFHDICGPNKELDEETCQCVCRAGLRPASCGPHKELDRNSCQCVCKNKLFPSQCGANREFDENTCQCVCKRTCPRNQPLNPGKCACECTESPQKCLLKGKKFHHQTCSCYRRPCTNRQKACEPGFSYSEEVCRCVPSYWKRPQMS

SEQ ID NO: 25 - DNA, 2029 bp, Homo sapiens
  CDS (411)..(1475)
gttgggttccagctttctgtagctgtaagcattggtggccacaccacctccttacaaagcaactagaacctgcggcatacattggagagatttttttaattttctggacatgaagtaaatttagagtgctttctaatttcaggtagaagacatgtccaccttctgattatttttggagaacattttgatttttttcatctctctctccccacccctaagattgtgcaaaaaaagcgtaccttgcctaattgaaataatttcattggattttgatcagaactgattatttggttttctgtgtgaagttttgaggtttcaaactttccttctggagaatgccttttgaaacaattttctctagctgcctgatgtcaactgcttagtaatcagtggatattgaaatattcaaaatgtacagagagtgggtagtggtgaatgttttcatgatgttgtacgtccagctggtgcagggctccagtaatgaacatggaccagtgaagcgatcatctcagtccacattggaacgatctgaacagcagatcagggctgcttctagtttggaggaactacttcgaattactcactctgaggactggaagctgtggagatgcaggctgaggctcaaaagttttaccagtatggactctcgctcagcatcccatcggtccactaggtttgcggcaactttctatgacattgaaacactaaaagttatagatgaagaatggcaaagaactcagtgcagccctagagaaacgtgcgtggaggtggccagtgagctggggaagagtaccaacacattcttcaagcccccttgtgtgaacgtgttccgatgtggtggctgttgcaatgaagagagccttatctgtatgaacaccagcacctcgtacatttccaaacagctctttgagatatcagtgcctttgacatcagtacctgaattagtgcctgttaaagttgccaatcatacaggttgtaagtgcttgccaacagccccccgccatccatactcaattatcagaagatccatccagatccctgaagaagatcgctgttcccattccaagaaactctgtcctattgacatgctatgggatagcaacaaatgtaaatgtgttttgcaggaggaaaatccacttgctggaacagaagaccactctcatctccaggaaccagctctctgtgggccacacatgatgtttgacgaagatcgttgcgagtgtgtctgtaaaacaccatgtcccaaagatctaatccagcaccccaaaaactgcagttgctttgagtgcaaagaaagtctggagacctgctgccagaagcacaagctatttcacccagacacctgcagctgtgaggacagatgcccctttcataccagaccatgtgcaagtggcaaaacagcatgtgcaaagcattgccgctttccaaaggagaaaagggctgcccaggggccccacagccgaaagaatccttgattcagcgttccaagttccccatccctgtcatttttaacagcatgctgctttgccaagttgctgtcactgtttttttcccaggtgttaaaaaaaaaatccattttacacagcaccacagtgaatccagaccaaccttccattcacaccagctaaggagtccctggttcattgatggatgtcttctagctgcagatgcctctgcgcaccaaggaatggagaggaggggacccatgtaatccttttgtttagttttgtttttgttttttggtgaatgagaaaggtgtgctggtcatggaatggcaggtgtcatatgactgattactcagagcagatgaggaaaactgtagtctctgagtcctttgctaatcgcaactcttgtgaattattctgattcttttttatgcagaatttgattcgtatgatcagtactgactttctgattactgtccagcttatagtcttccagtttaatgaactaccatctgatgtttcatatttaagtgtatttaaagaaaataaacaccattattcaagccaaaaaaaaaaaaaaaaaa

SEQ ID NO: 26 - protein, 354 aa, Homo sapiens
MYREWVVVNVFMMLYVQLVQGSSNEHGPVKRSSQSTLERSEQQIRAASSLEELLRITHSEDWKLWRCRLRLKSFTSMDSRSASHRSTRFAATFYDIETLKVIDEEWQRTQCSPRETCVEVASELGKSTNTFFKPPCVNVFRCGGCCNEESLICMNTSTSYISKQLFEISVPLTSVPELVPVKVANHTGCKCLPTAPRHPYSIIRRSIQIPEEDRCSHSKKLCPIDMLWDSNKCKCVLQEENPLAGTEDHSHLQEPALCGPHMMFDEDRCECVCKTPCPKDLIQHPKNCSCFECKESLETCCQKHKLFHPDTCSCEDRCPFHTRPCASGKTACAKHCRFPKEKRAAQGPHSRKNP

SEQ ID NO: 27 - DNA, 1645 bp, Homo sapiens
  CDS (322)..(771)
gggattcgggccgcccagctacgggaggacctggagtggcactgggcgcccgacggaccatccccgggacccgcctgcccctcggcgccccgccccgccgggccgctccccgtcgggttccccagccacagccttacctacgggctcctgactccgcaaggcttccagaagatgctcgaaccaccggccggggcctcggggcagcagtgagggaggcgtccagccccccactcagctcttctcctcctgtgccaggggctccccgggggatgagcatggtggttttccctcggagccccctggctcgggacgtctgagaagatgccggtcatgaggctgttcccttgcttcctgcagctcctggccgggctggcgctgcctgctgtgcccccccagcagtgggccttgtctgctgggaacggctcgtcagaggtggaagtggtacccttccaggaagtgtggggccgcagctactgccgggcgctggagaggctggtggacgtcgtgtccgagtaccccagcgaggtggagcacatgttcagcccatcctgtgtctccctgctgcgctgcaccggctgctgcggcgatgagaatctgcactgtgtgccggtggagacggccaatgtcaccatgcagctcctaaagatccgttctggggaccggccctcctacgtggagctgacgttctctcagcacgttcgctgcgaatgccggcctctgcgggagaagatgaagccggaaaggtgcggcgatgctgttccccggaggtaacccaccccttggaggagagagaccccgcacccggctcgtgtatttattaccgtcacactcttcagtgactcctgctggtacctgccctctatttattagccaactgtttccctgctgaatgcctcgctcccttcaagacgaggggcagggaaggacaggaccctcaggaattcagtgccttcaacaacgtgagagaaagagagaagccagccacagacccctgggagcttccgctttgaaagaagcaagacacgtggcctcgtgaggggcaagctaggccccagaggccctggaggtctccaggggcctgcagaaggaaagaagggggccctgctacctgttcttgggcctcaggctctgcacagacaagcagcccttgctttcggagctcctgtccaaagtagggatgcggattctgctggggccgccacggcctggtggtgggaaggccggcagcgggcggaggggattcagccacttccccctcttcttctgaagatcagaacattcagctctggagaacagtggttgcctgggggcttttgccactccttgtcccccgtgatctcccctcacactttgccatttgcttgtactgggacattgttctttccggccgaggtgccaccaccctgcccccactaagagacacatacagagtgggccccgggctggagaaagagctgcctggatgagaaacagctcagccagtggggatgaggtcaccaggggaggagcctgtgcgtcccagctgaaggcagtggcaggggagcaggttccccaagggccctggcacccccacaagctgtccctgcagggccatctgactgccaagccagattctcttgaataaagtattctagtgtggaaacgc

SEQ ID NO: 28 - protein, 149 aa, Homo sapiens
MPVMRLFPCFLQLLAGLALPAVPPQQWALSAGNGSSEVEVVPFQEVWGRSYCRALERLVDVVSEYPSEVEHMFSPSCVSLLRCTGCCGDENLHCVPVETANVTMQLLKIRSGDRPSYVELTFSQHVRCECRPLREKMKPERCGDAVPRR

SEQ ID NO: 29 - DNA, 4230 bp, Homo sapiens
  CDS (1)..(4065)
agcaaggtgctgctggccgtcgccctgtggctctgcgtggagacccgggccgcctctgtgggtttgcctagtgtttctcttgatctgcccaggctcagcatacaaaaagacatacttacaattaaggctaatacaactcttcaaattacttgcaggggacagagggacttggactggctttggcccaataatcagagtggcagtgagcaaagggtggaggtgactgagtgcagcgatggcctcttctgtaagacactcacaattccaaaagtgatcggaaatgacactggagcctacaagtgcttctaccgggaaactgacttggcctcggtcatttatgtctatgttcaagattacagatctccatttattgcttctgttagtgaccaacatggagtcgtgtacattactgagaacaaaaacaaaactgtggtgattccatgtctcgggtccatttcaaatctcaacgtgtcactttgtgcaagatacccagaaaagagatttgttcctgatggtaacagaatttcctgggacagcaagaagggctttactattcccagctacatgatcagctatgctggcatggtcttctgtgaagcaaaaattaatgatgaaagttaccagtctattatgtacatagttgtcgttgtagggtataggatttatgatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaaccttttgttgcttttggaagtggcatggaatctctggtggaagccacggtgggggagcgtgtcagaatccctgcgaagtaccttggttacccacccccagaaataaaatggtataaaaatggaataccccttgagtccaatcacacaattaaagcggggcatgtactgacgattatggaagtgagtgaaagagacacaggaaattacactgtcatccttaccaatcccatttcaaaggagaagcagagccatgtggtctctctggttgtgtatgtcccaccccagattggtgagaaatctctaatctctcctgtggattcctaccagtacggcaccactcaaacgctgacatgtacggtctatgccattcctcccccgcatcacatccactggtattggcagttggaggaagagtgcgccaacgagcccagccaagctgtctcagtgacaaacccatacccttgtgaagaatggagaagtgtggaggacttccagggaggaaataaaattgaagttaataaaaatcaatttgctctaattgaaggaaaaaacaaaactgtaagtacccttgttatccaagcggcaaatgtgtcagctttgtacaaatgtgaagcggtcaacaaagtcgggagaggagagagggtgatctccttccacgtgaccaggggtcctgaaattactttgcaacctgacatgcagcccactgagcaggagagcgtgtctttgtggtgcactgcagacagatctacgtttgagaacctcacatggtacaagcttggcccacagcctctgccaatccatgtgggagagttgcccacacctgtttgcaagaacttggatactctttggaaattgaatgccaccatgttctctaatagcacaaatgacattttgatcatggagcttaagaatgcatccttgcaggaccaaggagactatgtctgccttgctcaagacaggaagaccaagaaaagacattgcgtggtcaggcagctcacagtcctagagcgtgtggcacccacgatcacaggaaacctggagaatcagacgacaagtattggggaaagcatcgaagtctcatgcacggcatctgggaatccccctccacagatcatgtggtttaaagataatgagacccttgtagaagactcaggcattgtattgaaggatgggaaccggaacctcactatccgcagagtgaggaaggaggacgaaggcctctacacctgccaggcatgcagtgttcttggctgtgcaaaagtggaggcatttttcataatagaaggtgcccaggaaaagacgaacttggaaatcattattctagtaggcacgacggtgattgccatgttcttctggctacttcttgtcatcatcctagggaccgttaagcgggccaatggaggggaactgaagacaggctacttgtccatcgtcatggatccagatgaactcccattggatgaacattgtgaacgactgccttatgatgccagcaaatgggaattccccagagaccggctgaacctaggtaagcctcttggccgtggtgcctttggccaagagattgaagcagatgcctttggaattgacaagacagcaacttgcaggacagtagcagtcaaaatgttgaaagaaggagcaacacacagtgagcatcgagctctcatgtctgaactcaagatcctcattcatattggtcaccatctcaatgtggtcaaccttctaggtgcctgtaccaagccaggagggccactcatggtgattgtggaattctgcaaatttggaaacctgtccacttacctgaggagcaagagaaatgaatttgtcccctacaagaccaaaggggcacgattccgtcaagggaaagactacgttggagcaatccctgtggatctgaaacggcgcttggacagcatcaccagtagccagagctcagccagctctggatttgtggaggagaagtccctcagtgatgtagaagaagaggaagctcctgaagatctgtataaggacttcctgaccttggagcatctcatctgttacagcttccaagtggctaagggcatggagttcttggcatcgcgaaagtgtatccacagggacctggcggcacgaaatatcctcttatcggagaagaacgtggttaaaatctgtgactttggcttggcccgggatatttataaagatccagattatgtcagaaaaggagatgctcgcctccctttgaaatggatggccccagaaacaatttttgacagagtgtacacaatccagagtgacgtctggtcttttggtgttttgctgtgggaaatattttccttaggtgcttctccatatcctggggtaaagattgatgaagaattttgtaggcgattgaaagaaggaactagaatgagggcccctgattatactacaccagaaatgtaccagaccatgctggactgctggcacggggagcccagtcagagacccacgttttcagagttggtggaacatttgggaaatctcttgcaagctaatgctcagcaggatggcaaagactacattgttcttccgatatcagagactttgagcatggaagaggattctggactctctctgcctacctcacctgtttcctgtatggaggaggaggaagtatgtgaccccaaattccattatgacaacacagcaggaatcagtcagtatctgcagaacagtaagcgaaagagccggcctgtgagtgtaaaaacatttgaagatatcccgttagaagaaccagaagtaaaagtaatcccagatgacaaccagacggacagtggtatggttcttgcctcagaagagctgaaaactttggaagacagaaccaaattatctccatcttttggtggaatggtgcccagcaaaagcagggagtctgtggcatctgaaggctcaaaccagacaagcggctaccagtccggatatcactccgatgacacagacaccaccgtgtactccagtgaggaagcagaacttttaaagctgatagagattggagtgcaaaccggtagcacagcccagattctccagcctgacacggggaccacactgagctctcctcctgtttaaaaggaagcatccacaccccaactcccggacatcacatgagaggtctgctcagattttgaagtgttgttctttccaccagcaggaagtagccgcatttgattttcatttcgacaacagaaaaaggacctcggactgcagggagccagctcttctaggcttgtgacc

SEQ ID NO: 30 - protein, 1354 aa, Homo sapiens
SKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLEIIILVGTTVIAMFFWLLLVIILGTVKRANGGELKTGYLSIVMDPDELPLDEHCERLPYDASKWEFPRDRLNLGKPLGRGAFGQEIEADAFGIDKTATCRTVAVKMLKEGATHSEHRALMSELKILIHIGHHLNVVNLLGACTKPGGPLMVIVEFCKFGNLSTYLRSKRNEFVPYKTKGARFRQGKDYVGAIPVDLKRRLDSITSSQSSASSGFVEEKSLSDVEEEEAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANAQQDGKDYIVLPISETLSMEEDSGLSLPTSPVSCMEEEEVCDPKFHYDNTAGISQYLQNSKRKSRPVSVKTFEDIPLEEPEVKVIPDDNQTDSGMVLASEELKTLEDRTKLSPSFGGMVPSKSRESVASEGSNQTSGYQSGYHSDDTDTTVYSSEEAELLKLIEIGVQTGSTAQILQPDTGTTLSSPPV

SEQ ID NO: 31 - DNA, 4195 bp, Homo sapiens
  CDS (20)..(3913)
ccacgcgcagcggccggagatgcagcggggcgccgcgctgtgcctgcgactgtggctctgcctgggactcctggacggcctggtgagtggctactccatgacccccccgaccttgaacatcacggaggagtcacacgtcatcgacaccggtgacagcctgtccatctcctgcaggggacagcaccccctcgagtgggcttggccaggagctcaggaggcgccagccaccggagacaaggacagcgaggacacgggggtggtgcgagactgcgagggcacagacgccaggccctactgcaaggtgttgctgctgcacgaggtacatgccaacgacacaggcagctacgtctgctactacaagtacatcaaggcacgcatcgagggcaccacggccgccagctcctacgtgttcgtgagagactttgagcagccattcatcaacaagcctgacacgctcttggtcaacaggaaggacgccatgtgggtgccctgtctggtgtccatccccggcctcaatgtcacgctgcgctcgcaaagctcggtgctgtggccagacgggcaggaggtggtgtgggatgaccggcggggcatgctcgtgtccacgccactgctgcacgatgccctgtacctgcagtgcgagaccacctggggagaccaggacttcctttccaaccccttcctggtgcacatcacaggcaacgagctctatgacatccagctgttgcccaggaagtcgctggagctgctggtaggggagaagctggtcctgaactgcaccgtgtgggctgagtttaactcaggtgtcacctttgactgggactacccagggaagcaggcagagcggggtaagtgggtgcccgagcgacgctcccagcagacccacacagaactctccagcatcctgaccatccacaacgtcagccagcacgacctgggctcgtatgtgtgcaaggccaacaacggcatccagcgatttcgggagagcaccgaggtcattgtgcatgaaaatcccttcatcagcgtcgagtggctcaaaggacccatcctggaggccacggcaggagacgagctggtgaagctgcccgtgaagctggcagcgtaccccccgcccgagttccagtggtacaaggatggaaaggcactgtccgggcgccacagtccacatgccctggtgctcaaggaggtgacagaggccagcacaggcacctacaccctcgccctgtggaactccgctgctggcctgaggcgcaacatcagcctggagctggtggtgaatgtgcccccccagatacatgagaaggaggcctcctcccccagcatctactcgcgtcacagccgccaggccctcacctgcacggcctacggggtgcccctgcctctcagcatccagtggcactggcggccctggacaccctgcaagatgtttgcccagcgtagtctccggcggcggcagcagcaagacctcatgccacagtgccgtgactggagggcggtgaccacgcaggatgccgtgaaccccatcgagagcctggacacctggaccgagtttgtggagggaaagaataagactgtgagcaagctggtgatccagaatgccaacgtgtctgccatgtacaagtgtgtggtctccaacaaggtgggccaggatgagcggctcatctacttctatgtgaccaccatccccgacggcttcaccatcgaatccaagccatccgaggagctactagagggccagccggtgctcctgagctgccaagccgacagctacaagtacgagcatctgcgctggtaccgcctcaacctgtccacgctgcacgatgcgcacgggaacccgcttctgctcgactgcaagaacgtgcatctgttcgccacccctctggccgccagcctggaggaggtggcacctggggcgcgccacgccacgctcagcctgagtatcccccgcgtcgcgcccgagcacgagggccactatgtgtgcgaagtgcaagaccggcgcagccatgacaagcactgccacaagaagtacctgtcggtgcaggccctggaagcccctcggctcacgcagaacttgaccgacctcctggtgaacgtgagcgactcgctggagatgcagtgcttggtggccggagcgcacgcgcccagcatcgtgtggtacaaagacgagaggctgctggaggaaaagtctggagtcgacttggcggactccaaccagaagctgagcatccagcgcgtgcgcgaggaggatgcgggacgctatctgtgcagcgtgtgcaacgccaagggctgcgtcaactcctccgccagcgtggccgtggaaggctccgaggataagggcagcatggagatcgtgatccttgtcggtaccggcgtcatcgctgtcttcttctgggtcctcctcctcctcatcttctgtaacatgaggaggccggcccacgcagacatcaagacgggctacctgtccatcatcatggaccccggggaggtgcctctggaggagcaatgcgaatacctgtcctacgatgccagccagtgggaattcccccgagagcggctgcacctggggagagtgctcggctacggcgccttcgggaaggtggtggaagcctccgctttcggcatccacaagggcagcagctgtgacaccgtggccgtgaaaatgctgaaagagggcgccacggccagcgagcaccgcgcgctgatgtcggagctcaagatcctcattcacatcggcaaccacctcaacgtggtcaacctcctcggggcgtgcaccaagccgcagggccccctcatggtgatcgtggagttctgcaagtacggcaacctctccaacttcctgcgcgccaagcgggacgccttcagcccctgcgcggagaagtctcccgagcagcgcggacgcttccgcgccatggtggagctcgccaggctggatcggaggcggccggggagcagcgacagggtcctcttcgcgcggttctcgaagaccgagggcggagcgaggcgggcttctccagaccaagaagctgaggacctgtggctgagcccgctgaccatggaagatcttgtctgctacagcttccaggtggccagagggatggagttcctggcttcccgaaagtgcatccacagagacctggctgctcggaacattctgctgtcggaaagcgacgtggtgaagatctgtgactttggccttgcccgggacatctacaaagaccctgactacgtccgcaagggcagtgcccggctgcccctgaagtggatggcccctgaaagcatcttcgacaaggtgtacaccacgcagagtgacgtgtggtcctttggggtgcttctctgggagatcttctctctgggggcctccccgtaccctggggtgcagatcaatgaggagttctgccagcggctgagagacggcacaaggatgagggccccggagctggccactcccgccatacgccgcatcatgctgaactgctggtccggagaccccaaggcgagacctgcattctcggagctggtggagatcctgggggacctgctccagggcaggggcctgcaagaggaagaggaggtctgcatggccccgcgcagctctcagagctcagaagagggcagcttctcgcaggtgtccaccatggccctacacatcgcccaggctgacgctgaggacagcccgccaagcctgcagcgccacagcctggccgccaggtattacaactgggtgtcctttcccgggtgcctggccagaggggctgagacccgtggttcctccaggatgaagacatttgaggaattccccatgaccccaacgacctacaaaggctctgtggacaaccagacagacagtgggatggtgctggcctcggaggagtttgagcagatagagagcaggcatagacaagaaagcggcttcaggtagctgaagcagagagagagaaggcagcatacgtcagcattttcttctctgcacttataagaaagatcaaagactttaagactttcgctatttcttctactgctatctactacaaacttcaaagaggaaccaggaggacaagaggagcatgaaagtggacaaggagtgtgaccactgaagcaccacagggaaggggttaggcctccggatgactgcgggcaggcctggataatatccagcctcccacaagaagctggtggagcagagtgttccctgactcct

SEQ ID NO: 32 - protein, 1298 aa, Homo sapiens
MQRGAALCLRLWLCLGLLDGLVSGYSMTPPTLNITEESHVIDTGDSLSISCRGQHPLEWAWPGAQEAPATGDKDSEDTGVVRDCEGTDARPYCKVLLLHEVHANDTGSYVCYYKYIKARIEGTTAASSYVFVRDFEQPFINKPDTLLVNRKDAMWVPCLVSIPGLNVTLRSQSSVLWPDGQEVVWDDRRGMLVSTPLLHDALYLQCETTWGDQDFLSNPFLVHITGNELYDIQLLPRKSLELLVGEKLVLNCTVWAEFNSGVTFDWDYPGKQAERGKWVPERRSQQTHTELSSILTIHNVSQHDLGSYVCKANNGIQRFRESTEVIVHENPFISVEWLKGPILEATAGDELVKLPVKLAAYPPPEFQWYKDGKALSGRHSPHALVLKEVTEASTGTYTLALWNSAAGLRRNISLELVVNVPPQIHEKEASSPSIYSRHSRQALTCTAYGVPLPLSIQWHWRPWTPCKMFAQRSLRRRQQQDLMPQCRDWRAVTTQDAVNPIESLDTWTEFVEGKNKTVSKLVIQNANVSAMYKCVVSNKVGQDERLIYFYVTTIPDGFTIESKPSEELLEGQPVLLSCQADSYKYEHLRWYRLNLSTLHDAHGNPLLLDCKNVHLFATPLAASLEEVAPGARHATLSLSIPRVAPEHEGHYVCEVQDRRSHDKHCHKKYLSVQALEAPRLTQNLTDLLVNVSDSLEMQCLVAGAHAPSIVWYKDERLLEEKSGVDLADSNQKLSIQRVREEDAGRYLCSVCNAKGCVNSSASVAVEGSEDKGSMEIVILVGTGVIAVFFWVLLLLIFCNMRRPAHADIKTGYLSIIMDPGEVPLEEQCEYLSYDASQWEFPRERLHLGRVLGYGAFGKVVEASAFGIHKGSSCDTVAVKMLKEGATASEHRALMSELKILIHIGNHLNVVNLLGACTKPQGPLMVIVEFCKYGNLSNFLRAKRDAFSPCAEKSPEQRGRFRAMVELARLDRRRPGSSDRVLFARFSKTEGGARRASPDQEAEDLWLSPLTMEDLVCYSFQVARGMEFLASRKCIHRDLAARNILLSESDVVKICDFGLARDIYKDPDYVRKGSARLPLKWMAPESIFDKVYTTQSDVWSFGVLLWEIFSLGASPYPGVQINEEFCQRLRDGTRMRAPELATPAIRRIMLNCWSGDPKARPAFSELVEILGDLLQGRGLQEEEEVCMAPRSSQSSEEGSFSQVSTMALHIAQADAEDSPPSLQRHSLAARYYNWVSFPGCLARGAETRGSSRMKTFEEFPMTPTTYKGSVDNQTDSGMVLASEEFEQIESRHRQESGFR

SEQ ID NO: 33 - protein, 14 aa, Homo sapiens
GKPIPNPLLGLDST

SEQ ID NO: 34 - DNA, 18 bp, Artificial sequence
  Synthetic primer
tccggtttcctgtgaggc

SEQ ID NO: 35 - DNA, 18 bp, Artificial sequence
  Synthetic primer
aagttgggtaacgccagg

SEQ ID NO: 36 - DNA, 15 bp, Artificial sequence
  Synthetic primer
tgacctcgcccccgt

SEQ ID NO: 37 - DNA, 3088 bp, Homo sapiens
ccccttttccagaatcacttgcactgtcttgttcttgaatgagaaaggaagaaaagagcctcccattactcagacccgtgtaaacattattccccccaggagaaaatggtgttattcaaatgaatcataataaaatagcctctaaacagtttctaagcgggagcctccgtggaactcagcgctccgctcctcccagttcctaagaggtcccgggattcttgagctgtgcccagctgacgagcttttgaagatggcacaataaccgtccagtgatgcctgaccatgacagcacagccctcttaagccggcaaaccaagaggagaagagttgacattggagtgaaaaggacggtagggacagcatctgcattttttgctaaggcaagagcaacgttttttagtgccatgaatccccaaggttctgagcaggatgttgagtattcagtggtgcagcatgcagatggggaaaagtcaaatgtactccgcaagctgctgaagagggcgaactcgtatgaagatgccatgatgccttttccaggagcaaccataatttcccagctgttgaaaaataacatgaacaaaaatggtggcacggagcccagtttccaagccagcggtctctctagtacaggctccgaagtacatcaggaggatatatgcagcaactcttcaagagacagccccccagagtgtctttccccttttggcaggcctactatgagccagtttgatatggatcgcttatgtgatgagcacctgagagcaaagcgcgcccgggttgagaatataattcggggtatgagccattcccccagtgtggcattaaggggcaatgaaaatgaaagagagatggccccgcagtctgtgagtccccgagaaagttacagagaaaacaaacgcaagcaaaagcttccccagcagcagcaacagagtttccagcagctggtttcagcccgaaaagaacagaagcgagaggagcgccgacagctgaaacagcagctggaggacatgcagaaacagctgcgccagctgcaggaaaagttctaccaaatctatgacagcactgattcggaaaatgatgaagatggtaacctgtctgaagacagcatgcgctcggagatcctggatgccagggcccaggactctgtcggaaggtcagataatgagatgtgcgagctagacccaggacagtttattgaccgagctcgagccctgatcagagagcaggaaatggctgaaaacaagccgaagcgagaaggcaacaacaaagaaagagaccatgggccaaactccttacaaccggaaggcaaacatttggctgagaccttgaaacaggaactgaacactgccatgtcgcaagttgtggacactgtggtcaaagtcttttcggccaagccctcccgccaggttcctcaggtcttcccacctctccagatcccccaggccagatttgcagtcaatggggaaaaccacaatttccacaccgccaaccagcgcctgcagtgctttggcgacgtcatcattccgaaccccctggacacctttggcaatgtgcagatggccagttccactgaccagacagaagcactgcccctggttgtccgcaaaaactcctctgaccagtctgcctccggccctgccgctggcggccaccaccagcccctgcaccagtcgcctctctctgccaccacgggcttcaccacgtccaccttccgccaccccttcccccttcccttgatggcctatccatttcagagcccattaggtgctccctccggctccttctctggaaaagacagagcctctcctgaatccttagacttaactagggataccacgagtctgaggaccaagatgtcatctcaccacctgagccaccacccttgttcaccagcacacccgcccagcaccgccgaagggctctccttgtcgctcataaagtccgagtgcggcgatcttcaagatatgtctgaaatatcaccttattcgggaagtgcaatgcaggaaggattgtcacccaatcacttgaaaaaagcaaagctcatgtttttttatacccgttatcccagctccaatatgctgaagacctacttctccgacgtaaagttcaacagatgcattacctctcagctcatcaagtggtttagcaatttccgtgagttttactacattcagatggagaagtacgcacgtcaagccatcaacgatggggtcaccagtactgaagagctgtctataaccagagactgtgagctgtacagggctctgaacatgcactacaataaagcaaatgactttgaggttccagagagattcctggaagttgctcagatcacattacgggagtttttcaatgccattatcgcaggcaaagatgttgatccttcctggaagaaggccatatacaaggtcatctgcaagctggatagtgaagtccctgagattttcaaatccccgaactgcctacaagagctgcttcatgagtagaaatttcaacaactctttttgaatgtatgaagagtagcagtcccctttggatgtccaagttatatgtgtctagattttgatttcatatatatgtgtatgggaggcatggatatgttatgaaatcagctggtaattcctcctcatcacgtttctctcattttcttttgttttccattgcaaggggatggttgttttctttctgcctttagtttgcttttgcccaaggcccttaacatttggacacttaaaatagggttaattttcagggaaaaagaatgttggcgtgtgtaaagtctctattagcaatgaagggaatttgttaacgatgcatccacttgattgatgacttattgcaaatggcggttggctgaggaaaacccatgacacagcacaactctacagacagtgatgtgtctcttgtttctactgctaagaaggtctgaaaatttaatgaaaccacttcatacatttaagtattttgtttggtttgaactcaatcagtagcttttccttacatgtttaaaaataattccaatgacagatgagcagctcacttttccaaagtaccccaaaaggccaaattaaaaaaaaaaaaaaaaaaa

SEQ ID NO: 38 - protein, 737 aa, Homo sapiens
MPDHDSTALLSRQTKRRRVDIGVKRTVGTASAFFAKARATFFSAMNPQGSEQDVEYSVVQHADGEKSNVLRKLLKRANSYEDAMMPFPGATIISQLLKNNMNKNGGTEPSFQASGLSSTGSEVHQEDICSNSSRDSPPECLSPFGRPTMSQFDMDRLCDEHLRAKRARVENIIRGMSHSPSVALRGNENEREMAPQSVSPRESYRENKRKQKLPQQQQQSFQQLVSARKEQKREERRQLKQQLEDMQKQLRQLQEKFYQIYDSTDSENDEDGNLSEDSMRSEILDARAQDSVGRSDNEMCELDPGQFIDRARALIREQEMAENKPKREGNNKERDHGPNSLQPEGKHLAETLKQELNTAMSQVVDTVVKVFSAKPSRQVPQVFPPLQIPQARFAVNGENHNFHTANQRLQCFGDVIIPNPLDTFGNVQMASSTDQTEALPLVVRKNSSDQSASGPAAGGHHQPLHQSPLSATTGFTTSTFRHPFPLPLMAYPFQSPLGAPSGSFSGKDRASPESLDLTRDTTSLRTKMSSHHLSHHPCSPAHPPSTAEGLSLSLIKSECGDLQDMSEISPYSGSAMQEGLSPNHLKKAKLMFFYTRYPSSNMLKTYFSDVKFNRCITSQLIKWFSNFREFYYIQMEKYARQAINDGVTSTEELSITRDCELYRALNMHYNKANDFEVPERFLEVAQITLREFFNAIIAGKDVDPSWKKAIYKVICKLDSEVPEIFKSPNCLQELLHE